SEQUENCE LISTING (11 sequences)

SEQ ID NO: 1 - DNA, 2610 bp, homo sapiens
atgggggcccggagcggagctcggggcgcgctgctgctggcactgctgctctgctgggacccgaggctgagccaagcaggcactgattctggcagcgaggtgctccctgactccttcccgtcagcgccagcagagccgctgccctacttcctgcaggagccacaggacgcctacattgtgaagaacaagcctgtggagctccgctgccgcgccttccccgccacacagatctacttcaagtgcaacggcgagtgggtcagccagaacgaccacgtcacacaggaaggcctggatgaggccaccggcctgcgggtgcgcgaggtgcagatcgaggtgtcgcggcagcaggtggaggagctctttgggctggaggattactggtgccagtgcgtggcctggagctccgcaggcaccaccaagagtcgccgagcctacgtccgcatcgcctacctgcgcaagaacttcgatcaggagcctctgggcaaggaggtgcccctggaccatgaggttctcctgcagtgccgcccgccggagggggtgcctgtggccgaggtggaatggctcaagaatgaggatgtcatcgaccccacccaggacaccaacttcctgctcaccatcgaccacaacctcatcatccgccaggcccgcctgtcggacactgccaactatacctgcgtggccaagaacatcgtggccaaacgccggagcaccactgccaccgtcatcgtctacgtgaatggcggctggtccagctgggcagagtggtcaccctgctccaaccgctgtggccgaggctggcagaagcgcacccggacctgcaccaaccccgctccactcaacggaggggccttctgcgagggccaggcattccagaagaccgcctgcaccaccatctgcccagtcgatggggcgtggacggagtggagcaagtggtcagcctgcagcactgagtgtgcccactggcgtagccgcgagtgcatggcgcccccaccccagaacggaggccgtgactgcagcgggacgctgctcgactctaagaactgcacagatgggctgtgcatgcaaagcaacccgcagctcctacacccctctgtgcctcctgacctgacagccagcgccggcatctaccgcggacccgtgtatgccctgcaggactccaccgacaaaatccccatgaccaactctcctctgctggaccccttacccagccttaaggtcaaggtctacagctccagcaccacgggctctgggccaggcctggcagatggggctgacctgctgggggtcttgccgcctggcacataccctagcgatttcgcccgggacacccacttcctgcacctgcgcagcgccagcctcggttcccagcagctcttgggcctgccccgagacccagggagcagcgtcagcggcacctttggctgcctgggtgggaggctcagcatccccggcacaggggtcagcttgctggtgcccaatggagccattccccagggcaagttctacgagatgtatctactcatcaacaaggcagaaagtaccctcccgctttcagaagggacccagacagtattgagcccctcggtgacctgtggacccacaggcctcctgctgtgccgccccgtcatcctcaccatgccccactgtgccgaagtcagtgcccgtgactggatctttcagctcaagacccaggcccaccagggccactgggaggaggtggtgaccctggatgaggagaccctgaacacaccctgctactgccagctggagcccagggcctgtcacatcctgctggaccagctgggcacctacgtgttcacgggcgagtcctattcccgctcagcagtcaagcggctccagctggccgtcttcgcccccgccctctgcacctccctggagtacagcctccgggtctactgcctggaggacacgcctgtagcactgaaggaggtgctggagctggagcggactctgggcggatacttggtggaggagccgaaaccgctaatgttcaaggacagttaccacaacctgcgcctctccctccatgacctcccccatgcccattggaggagcaagctgctggccaaataccaggagatccccttctatcacatttggagtggcagccagaaggccctccactgcactttcaccctggagaggcacagcttggcctccacagagctcacctgcaagatctgcgtgcggcaagtggaaggggagggccagatattccagctgcataccactctggcagagacacctgctggctccctggacactctctgctctgcccctggcagcactgtcaccacccagctgggaccttatgccttcaagatcccactgtccatccgccagaagatatgcaacagcctagatgcccccaactcacggggcaatgactggcggatgttagcacagaagctctctatggaccggtacctgaattactttgccaccaaagcgagccccacgggtgtgatcctggacctctgggaagctctgcagcaggacgatggggacctcaacagcctggcgagtgccttggaggagatgggcaagagtgagatgctggtggctgtggccaccgacggggactgctga

SEQ ID NO: 2 - protein, 869 aa, homo sapiens
MGARSGARGALLLALLLCWDPRLSQAGTDSGSEVLPDSFPSAPAEPLPYFLQEPQDAYIVKNKPVELRCRAFPATQIYFKCNGEWVSQNDHVTQEGLDEATGLRVREVQIEVSRQQVEELFGLEDYWCQCVAWSSAGTTKSRRAYVRIAYLRKNFDQEPLGKEVPLDHEVLLQCRPPEGVPVAEVEWLKNEDVIDPTQDTNFLLTIDHNLIIRQARLSDTANYTCVAKNIVAKRRSTTATVIVYVNGGWSSWAEWSPCSNRCGRGWQKRTRTCTNPAPLNGGAFCEGQAFQKTACTTICPVDGAWTEWSKWSACSTECAHWRSRECMAPPPQNGGRDCSGTLLDSKNCTDGLCMQSNPQLLHPSVPPDLTASAGIYRGPVYALQDSTDKIPMTNSPLLDPLPSLKVKVYSSSTTGSGPGLADGADLLGVLPPGTYPSDFARDTHFLHLRSASLGSQQLLGLPRDPGSSVSGTFGCLGGRLSIPGTGVSLLVPNGAIPQGKFYEMYLLINKAESTLPLSEGTQTVLSPSVTCGPTGLLLCRPVILTMPHCAEVSARDWIFQLKTQAHQGHWEEVVTLDEETLNTPCYCQLEPRACHILLDQLGTYVFTGESYSRSAVKRLQLAVFAPALCTSLEYSLRVYCLEDTPVALKEVLELERTLGGYLVEEPKPLMFKDSYHNLRLSLHDLPHAHWRSKLLAKYQEIPFYHIWSGSQKALHCTFTLERHSLASTELTCKICVRQVEGEGQIFQLHTTLAETPAGSLDTLCSAPGSTVTTQLGPYAFKIPLSIRQKICNSLDAPNSRGNDWRMLAQKLSMDRYLNYFATKASPTGVILDLWEALQQDDGDLNSLASALEEMGKSEMLVAVATDGDC

SEQ ID NO: 3 - DNA, 2532 bp, homo sapiens
ggcactgattctggcagcgaggtgctccctgactccttcccgtcagcgccagcagagccgctgccctacttcctgcaggagccacaggacgcctacattgtgaagaacaagcctgtggagctccgctgccgcgccttccccgccacacagatctacttcaagtgcaacggcgagtgggtcagccagaacgaccacgtcacacaggaaggcctggatgaggccaccggcctgcgggtgcgcgaggtgcagatcgaggtgtcgcggcagcaggtggaggagctctttgggctggaggattactggtgccagtgcgtggcctggagctccgcaggcaccaccaagagtcgccgagcctacgtccgcatcgcctacctgcgcaagaacttcgatcaggagcctctgggcaaggaggtgcccctggaccatgaggttctcctgcagtgccgcccgccggagggggtgcctgtggccgaggtggaatggctcaagaatgaggatgtcatcgaccccacccaggacaccaacttcctgctcaccatcgaccacaacctcatcatccgccaggcccgcctgtcggacactgccaactatacctgcgtggccaagaacatcgtggccaaacgccggagcaccactgccaccgtcatcgtctacgtgaatggcggctggtccagctgggcagagtggtcaccctgctccaaccgctgtggccgaggctggcagaagcgcacccggacctgcaccaaccccgctccactcaacggaggggccttctgcgagggccaggcattccagaagaccgcctgcaccaccatctgcccagtcgatggggcgtggacggagtggagcaagtggtcagcctgcagcactgagtgtgcccactggcgtagccgcgagtgcatggcgcccccaccccagaacggaggccgtgactgcagcgggacgctgctcgactctaagaactgcacagatgggctgtgcatgcaaagcaacccgcagctcctacacccctctgtgcctcctgacctgacagccagcgccggcatctaccgcggacccgtgtatgccctgcaggactccaccgacaaaatccccatgaccaactctcctctgctggaccccttacccagccttaaggtcaaggtctacagctccagcaccacgggctctgggccaggcctggcagatggggctgacctgctgggggtcttgccgcctggcacataccctagcgatttcgcccgggacacccacttcctgcacctgcgcagcgccagcctcggttcccagcagctcttgggcctgccccgagacccagggagcagcgtcagcggcacctttggctgcctgggtgggaggctcagcatccccggcacaggggtcagcttgctggtgcccaatggagccattccccagggcaagttctacgagatgtatctactcatcaacaaggcagaaagtaccctcccgctttcagaagggacccagacagtattgagcccctcggtgacctgtggacccacaggcctcctgctgtgccgccccgtcatcctcaccatgccccactgtgccgaagtcagtgcccgtgactggatctttcagctcaagacccaggcccaccagggccactgggaggaggtggtgaccctggatgaggagaccctgaacacaccctgctactgccagctggagcccagggcctgtcacatcctgctggaccagctgggcacctacgtgttcacgggcgagtcctattcccgctcagcagtcaagcggctccagctggccgtcttcgcccccgccctctgcacctccctggagtacagcctccgggtctactgcctggaggacacgcctgtagcactgaaggaggtgctggagctggagcggactctgggcggatacttggtggaggagccgaaaccgctaatgttcaaggacagttaccacaacctgcgcctctccctccatgacctcccccatgcccattggaggagcaagctgctggccaaataccaggagatccccttctatcacatttggagtggcagccagaaggccctccactgcactttcaccctggagaggcacagcttggcctccacagagctcacctgcaagatctgcgtgcggcaagtggaaggggagggccagatattccagctgcataccactctggcagagacacctgctggctccctggacactctctgctctgcccctggcagcactgtcaccacccagctgggaccttatgccttcaagatcccactgtccatccgccagaagatatgcaacagcctagatgcccccaactcacggggcaatgactggcggatgttagcacagaagctctctatggaccggtacctgaattactttgccaccaaagcgagccccacgggtgtgatcctggacctctgggaagctctgcagcaggacgatggggacctcaacagcctggcgagtgccttggaggagatgggcaagagtgagatgctggtggctgtggccaccgacggggactgctga

SEQ ID NO: 4 - protein, 843 aa, homo sapiens
GTDSGSEVLPDSFPSAPAEPLPYFLQEPQDAYIVKNKPVELRCRAFPATQIYFKCNGEWVSQNDHVTQEGLDEATGLRVREVQIEVSRQQVEELFGLEDYWCQCVAWSSAGTTKSRRAYVRIAYLRKNFDQEPLGKEVPLDHEVLLQCRPPEGVPVAEVEWLKNEDVIDPTQDTNFLLTIDHNLIIRQARLSDTANYTCVAKNIVAKRRSTTATVIVYVNGGWSSWAEWSPCSNRCGRGWQKRTRTCTNPAPLNGGAFCEGQAFQKTACTTICPVDGAWTEWSKWSACSTECAHWRSRECMAPPPQNGGRDCSGTLLDSKNCTDGLCMQSNPQLLHPSVPPDLTASAGIYRGPVYALQDSTDKIPMTNSPLLDPLPSLKVKVYSSSTTGSGPGLADGADLLGVLPPGTYPSDFARDTHFLHLRSASLGSQQLLGLPRDPGSSVSGTFGCLGGRLSIPGTGVSLLVPNGAIPQGKFYEMYLLINKAESTLPLSEGTQTVLSPSVTCGPTGLLLCRPVILTMPHCAEVSARDWIFQLKTQAHQGHWEEVVTLDEETLNTPCYCQLEPRACHILLDQLGTYVFTGESYSRSAVKRLQLAVFAPALCTSLEYSLRVYCLEDTPVALKEVLELERTLGGYLVEEPKPLMFKDSYHNLRLSLHDLPHAHWRSKLLAKYQEIPFYHIWSGSQKALHCTFTLERHSLASTELTCKICVRQVEGEGQIFQLHTTLAETPAGSLDTLCSAPGSTVTTQLGPYAFKIPLSIRQKICNSLDAPNSRGNDWRMLAQKLSMDRYLNYFATKASPTGVILDLWEALQQDDGDLNSLASALEEMGKSEMLVAVATDGDC

SEQ ID NO: 5 - protein, 934 aa, homo sapiens
MGARSGARGALLLALLLCWDPRLSQAGTDSGSEVLPDSFPSAPAEPLPYFLQEPQDAYIVKNKPVELRCRAFPATQIYFKCNGEWVSQNDHVTQEGLDEATGLRVREVQIEVSRQQVEELFGLEDYWCQCVAWSSAGTTKSRRAYVRIAYLRKNFDQEPLGKEVPLDHEVLLQCRPPEGVPVAEVEWLKNEDVIDPTQDTNFLLTIDHNLIIRQARLSDTANYTCVAKNIVAKRRSTTATVIVYVNGGWSSWAEWSPCSNRCGRGWQKRTRTCTNPAPLNGGAFCEGQAFQKTACTTICPVDGAWTEWSKWSACSTECAHWRSRECMAPPPQNGGRDCSGTLLDSKNCTDGLCMQMLEASGDAALYAGLVVAIFVVVAILMAVGVVVYRRNCRDFDTDITDSSAALTGGFHPVNFKTARPSNPQLLHPSVPPDLTASAGIYRGPVYALQDSTDKIPMTNSPLLDPLPSLKVKVYSSSTTGSGPGLADGADLLGVLPPGTYPSDFARDTHFLHLRSASLGSQQLLGLPRDPGSSVSGTFGCLGGRLSIPGTGVSLLVPNGAIPQGKFYEMYLLINKAESTLPLSEGTQTVLSPSVTCGPTGLLLCRPVILTMPHCAEVSARDWIFQLKTQAHQGHWEEVVTLDEETLNTPCYCQLEPRACHILLDQLGTYVFTGESYSRSAVKRLQLAVFAPALCTSLEYSLRVYCLEDTPVALKEVLELERTLGGYLVEEPKPLMFKDSYHNLRLSLHDLPHAHWRSKLLAKYQEIPFYHIWSGSQKALHCTFTLERHSLASTELTCKICVRQVEGEGQIFQLHTTLAETPAGSLDTLCSAPGSTVTTQLGPYAFKIPLSIRQKICNSLDAPNSRGNDWRMLAQKLSMDRYLNYFATKASPTGVILDLWEALQQDDGDLNSLASALEEMGKSEMLVAVATDGDC

SEQ ID NO: 6 - protein, 329 aa, homo sapiens
MAPPPQNGGRDCSGTLLDSKNCTDGLCMQNKKTLSDPNSHLLEASGDAALYAGLVVAIFVVVAILMAVGVVVYRRNCRDFDTDITDSSAALTGGFHPVNFKTARPSNPQLLHPSVPPDLTASAGIYRGPVYALQDSTDKIPMTNSPLLDPLPSLKVKVYSSSTTGSGPGLADGADLLGVLPPGTYPSDFARDTHFLHLRSASLGSQQLLGLPRDPGSSVSGTFGCLGGRLSIPGTGVSLLVPNGAIPQGKFYEMYLLINKAESTLPLSEGTQTVLSPSVTCGPTGLLLCRPVILTMPHCAEVSARDWIFQLKTQAHQGHWEVRSHGEGW

SEQ ID NO: 7 - protein, 945 aa, homo sapiens
MGARSGARGALLLALLLCWDPRLSQAGTDSGSEVLPDSFPSAPAEPLPYFLQEPQDAYIVKNKPVELRCRAFPATQIYFKCNGEWVSQNDHVTQEGLDEATGLRVREVQIEVSRQQVEELFGLEDYWCQCVAWSSAGTTKSRRAYVRIAYLRKNFDQEPLGKEVPLDHEVLLQCRPPEGVPVAEVEWLKNEDVIDPTQDTNFLLTIDHNLIIRQARLSDTANYTCVAKNIVAKRRSTTATVIVYVNGGWSSWAEWSPCSNRCGRGWQKRTRTCTNPAPLNGGAFCEGQAFQKTACTTICPVDGAWTEWSKWSACSTECAHWRSRECMAPPPQNGGRDCSGTLLDSKNCTDGLCMQNKKTLSDPNSHLLEASGDAALYAGLVVAIFVVVAILMAVGVVVYRRNCRDFDTDITDSSAALTGGFHPVNFKTARPSNPQLLHPSVPPDLTASAGIYRGPVYALQDSTDKIPMTNSPLLDPLPSLKVKVYSSSTTGSGPGLADGADLLGVLPPGTYPSDFARDTHFLHLRSASLGSQQLLGLPRDPGSSVSGTFGCLGGRLSIPGTGVSLLVPNGAIPQGKFYEMYLLINKAESTLPLSEGTQTVLSPSVTCGPTGLLLCRPVILTMPHCAEVSARDWIFQLKTQAHQGHWEEVVTLDEETLNTPCYCQLEPRACHILLDQLGTYVFTGESYSRSAVKRLQLAVFAPALCTSLEYSLRVYCLEDTPVALKEVLELERTLGGYLVEEPKPLMFKDSYHNLRLSLHDLPHAHWRSKLLAKYQEIPFYHIWSGSQKALHCTFTLERHSLASTELTCKICVRQVEGEGQIFQLHTTLAETPAGSLDTLCSAPGSTVTTQLGPYAFKIPLSIRQKICNSLDAPNSRGNDWRMLAQKLSMDRYLNYFATKASPTGVILDLWEALQQDDGDLNSLASALEEMGKSEMLVAVATDGDC

SEQ ID NO: 8 - DNA, 3770 bp, homo sapiens
gagactggggccagggagacagccctgggggagaggcgcccgaaccaggccgcgggagcatgggggcccggagcggagctcggggcgcgctgctgctggcactgctgctctgctgggacccgaggctgagccaagcaggcactgattctggcagcgaggtgctccctgactccttcccgtcagcgccagcagagccgctgccctacttcctgcaggagccacaggacgcctacattgtgaagaacaagcctgtggagctccgctgccgcgccttccccgccacacagatctacttcaagtgcaacggcgagtgggtcagccagaacgaccacgtcacacaggaaggcctggatgaggccaccggcctgcgggtgcgcgaggtgcagatcgaggtgtcgcggcagcaggtggaggagctctttgggctggaggattactggtgccagtgcgtggcctggagctccgcaggcaccaccaagagtcgccgagcctacgtccgcatcgcctacctgcgcaagaacttcgatcaggagcctctgggcaaggaggtgcccctggaccatgaggttctcctgcagtgccgcccgccggagggggtgcctgtggccgaggtggaatggctcaagaatgaggatgtcatcgaccccacccaggacaccaacttcctgctcaccatcgaccacaacctcatcatccgccaggcccgcctgtcggacactgccaactatacctgcgtggccaagaacatcgtggccaaacgccggagcaccactgccaccgtcatcgtctacgtgaatggcggctggtccagctgggcagagtggtcaccctgctccaaccgctgtggccgaggctggcagaagcgcacccggacctgcaccaaccccgctccactcaacggaggggccttctgcgagggccaggcattccagaagaccgcctgcaccaccatctgcccagtcgatggggcgtggacggagtggagcaagtggtcagcctgcagcactgagtgtgcccactggcgtagccgcgagtgcatggcgcccccaccccagaacggaggccgtgactgcagcgggacgctgctcgactctaagaactgcacagatgggctgtgcatgcaaatgctggaggcctcaggggatgcggcgctgtatgcggggctcgtggtggccatcttcgtggtcgtggcaatcctcatggcggtgggggtggtggtgtaccgccgcaactgccgtgacttcgacacagacatcactgactcatctgctgccctgactggtggtttccaccccgtcaactttaagacggcaaggcccagcaacccgcagctcctacacccctctgtgcctcctgacctgacagccagcgccggcatctaccgcggacccgtgtatgccctgcaggactccaccgacaaaatccccatgaccaactctcctctgctggaccccttacccagccttaaggtcaaggtctacagctccagcaccacgggctctgggccaggcctggcagatggggctgacctgctgggggtcttgccgcctggcacataccctagcgatttcgcccgggacacccacttcctgcacctgcgcagcgccagcctcggttcccagcagctcttgggcctgccccgagacccagggagcagcgtcagcggcacctttggctgcctgggtgggaggctcagcatccccggcacaggggtcagcttgctggtgcccaatggagccattccccagggcaagttctacgagatgtatctactcatcaacaaggcagaaagtaccctcccgctttcagaagggacccagacagtattgagcccctcggtgacctgtggacccacaggcctcctgctgtgccgccccgtcatcctcaccatgccccactgtgccgaagtcagtgcccgtgactggatctttcagctcaagacccaggcccaccagggccactgggaggaggtggtgaccctggatgaggagaccctgaacacaccctgctactgccagctggagcccagggcctgtcacatcctgctggaccagctgggcacctacgtgttcacgggcgagtcctattcccgctcagcagtcaagcggctccagctggccgtcttcgcccccgccctctgcacctccctggagtacagcctccgggtctactgcctggaggacacgcctgtagcactgaaggaggtgctggagctggagcggactctgggcggatacttggtggaggagccgaaaccgctaatgttcaaggacagttaccacaacctgcgcctctccctccatgacctcccccatgcccattggaggagcaagctgctggccaaataccaggagatccccttctatcacatttggagtggcagccagaaggccctccactgcactttcaccctggagaggcacagcttggcctccacagagctcacctgcaagatctgcgtgcggcaagtggaaggggagggccagatattccagctgcataccactctggcagagacacctgctggctccctggacactctctgctctgcccctggcagcactgtcaccacccagctgggaccttatgccttcaagatcccactgtccatccgccagaagatatgcaacagcctagatgcccccaactcacggggcaatgactggcggatgttagcacagaagctctctatggaccggtacctgaattactttgccaccaaagcgagccccacgggtgtgatcctggacctctgggaagctctgcagcaggacgatggggacctcaacagcctggcgagtgccttggaggagatgggcaagagtgagatgctggtggctgtggccaccgacggggactgctgagcctcctgggacagcgggctggcagggactggcaggaggcaggtgcagggaggcctggggcagcctcctgatggggatgtttggcctctgcttcctcccagttcacagccagagttgcctctcctcctcctcttccccaacccccagaccatgaccagccttagaaaatccatgtactctgttgttagagggcccagagttccttctccacccccgctctctctctcttggcctgagatctctgtgcaggaaccaagatggggctgaagcctctggaggcagttggttgggggcgggcaggcaggaagcccttccttcacccccccacccttagcccggaacttctgggttccatgggttttaagttccgttcttcgttttcttcctccgtattgattttctccttttttccctaagccccctttgcttccacccctttttctctttgagagtcaagtacaatttaaaacaaacttgcttttttcttttccaaaacaaaaaggcaaggaaaaaaagaaagcttcaaaccgtttgtaaggttaaagaaaaaaaaaaccctccacccccccaccctcagcccggcaacttctgggttccatgggttttagttccgttctcgttttcttcctccgttattgatttctcctttctccctaagcccccttctgcttccacgcccttttcctctttgaagagtcaagtacaattcagacaaactgctttctcctgtccaaaagcaaaaaggcaaaggaaagaaagaaagcttcaaaccgctagtaaggctcaaagaagaagaaaaacaccaaaaccacaagggaaaagaaaaacccagtttcttaggaaacgcaaacgatttattatccagattatttggataagtcctttttaagaaaaaaaaaaaaaaaa

SEQ ID NO: 9 - DNA, 1228 bp, homo sapiens
ggcggctggtccagctgggcagagtggtcaccctgctccaaccgctgtggccgaggctggcagaagcgcacccggacctgcaccaaccccgctccactcaacggaggggccttctgcgagggccaggcattccagaagaccgcctgcaccaccatctgcccagtcgatggggcgtggacggagtggagcaagtggtcagcctgcagcactgagtgtgcccactggcgtagccgcgagtgcatggcgcccccaccccagaacggaggccgtgactgcagcgggacgctgctcgactctaagaactgcacagatgggctgtgcatgcaaaataagaaaactctaagcgaccccaacagccacctgctggaggcctcaggggatgcggcgctgtatgcggggctcgtggtggccatcttcgtggtcgtggcaatcctcatggcggtgggggtggtggtgtaccgccgcaactgccgtgacttcgacacagacatcactgactcatctgctgccctgactggtggtttccaccccgtcaactttaagacggcaaggcccagcaacccgcagctcctacacccctctgtgcctcctgacctgacagccagcgccggcatctaccgcggacccgtgtatgccctgcaggactccaccgacaaaatccccatgaccaactctcctctgctggaccccttacccagccttaaggtcaaggtctacagctccagcaccacgggctctgggccaggcctggcagatggggctgacctgctgggggtcttgccgcctggcacataccctagcgatttcgcccgggacacccacttcctgcacctgcgcagcgccagcctcggttcccagcagctcttgggcctgccccgagacccagggagcagcgtcagcggcacctttggctgcctgggtgggaggctcagcatccccggcacaggggtcagcttgctggtgcccaatggagccattccccagggcaagttctacgagatgtatctactcatcaacaaggcagaaagtaccctcccgctttcagaagggacccagacagtattgagcccctcggtgacctgtggacccacaggcctcctgctgtgccgccccgtcatcctcaccatgccccactgtgccgaagtcagtgcccgtgactggatctttcagctcaagacccaggcccaccagggccactgggaggtgaggagccacggtgaaggctggc

SEQ ID NO: 10 - DNA, 3935 bp, homo sapiens
cccacgcgtccgggctagggcgccggagccgcacgcagccgcggggctccgagaggcgcgcactggggctgggactgcgcggcgccgccgctgcgagcgccactgagcggtcgcgcaacttcggaggcacagcgccggagccaggcgagcgctcagagacccggagccagaggggcgcgccggagcctcgttcgagagccggcgccaggcacccaccgcgctccgagtgccaggcggccctccgcgcagcgtggcttccgctgcccccacggaaggcacgggctggcgctgccgggcgccggggaggacggcgaggaggaggcggcggcggcggagacggcggcggcgagactggggccagggagacagccctgggggagaggcgcccgaaccaggccgcgggagcatgggggcccggagcggagctcggggcgcgctgctgctggcactgctgctctgctgggacccgaggctgagccaagcaggcactgattctggcagcgaggtgctccctgactccttcccgtcagcgccagcagagccgctgccctacttcctgcaggagccacaggacgcctacattgtgaagaacaagcctgtggagctccgctgccgcgccttccccgccacacagatctacttcaagtgcaacggcgagtgggtcagccagaacgaccacgtcacacaggaaggcctggatgaggccaccggcctgcgggtgcgcgaggtgcagatcgaggtgtcgcggcagcaggtggaggagctctttgggctggaggattactggtgccagtgcgtggcctggagctccgcgggcaccaccaagagtcgccgagcctacgtccgcatcgcctacctgcgcaagaacttcgatcaggagcctctgggcaaggaggtgcccctggaccatgaggttctcctgcagtgccgcccgccggagggggtgcctgtggccgaggtggaatggctcaagaatgaggatgtcatcgaccccacccaggacaccaacttcctgctcaccatcgaccacaacctcatcatccgccaggcccgcctgtcggacactgccaactatacctgcgtggccaagaacatcgtggccaaacgccggagcaccactgccaccgtcatcgtctacgtgaatggcggctggtccagctgggcagagtggtcaccctgctccaaccgctgtggccgaggctggcagaagcgcacccggacctgcaccaaccccgctccactcaacggaggggccttctgcgagggccaggcattccagaagaccgcctgcaccaccatctgcccagtcgatggggcgtggacggagtggagcaagtggtcagcctgcagcactgagtgtgcccactggcgtagccgcgagtgcatggcgcccccaccccagaacggaggccgtgactgcagcgggacgctgctcgactctaagaactgcacagatgggctgtgcatgcaaaataagaaaactctaagcgaccccaacagccacctgctggaggcctcaggggatgcggcgctgtatgcggggctcgtggtggccatcttcgtggtcgtggcaatcctcatggcggtgggggtggtggtgtaccgccgcaactgccgtgacttcgacacagacatcactgactcatctgctgccctgactggtggtttccaccccgtcaactttaagacggcaaggcccagcaacccgcagctcctacacccctctgtgcctcctgacctgacagccagcgccggcatctaccgcggacccgtgtatgccctgcaggactccaccgacaaaatccccatgaccaactctcctctgctggaccccttacccagccttaaggtcaaggtctacagctccagcaccacgggctctgggccaggcctggcagacggggctgacctgctgggggtcttgccgcctggcacataccctagcgatttcgcccgggacacccacttcctgcacctgcgcagcgccagcctcggttcccagcagctcttgggcctgccccgagacccagggagcagcgtcagcggcacctttggctgcctgggtgggaggctcagcatccccggcacaggggtcagcttgctggtgcccaatggagccattccccagggcaagttctacgagatgtatctactcatcaacaaggcagaaagtaccctcccgctttcagaagggacccagacagtattgagcccctcggtgacctgtggacccacaggcctcctgctgtgccgccccgtcatcctcaccatgccccactgtgccgaagtcagtgcccgtgactggatctttcagctcaagacccaggcccaccagggccactgggaggaggtggtgaccctggatgaggagaccctgaacacaccctgctactgccagctggagcccagggcctgtcacatcctgctggaccagctgggcacctacgtgttcacgggcgagtcctattcccgctcagcagtcaagcggctccagctggccgtcttcgcccccgccctctgcacctccctggagtacagcctccgggtctactgcctggaggacacgcctgtagcactgaaggaggtgctggagctggagcggactctgggcggatacttggtggaggagccgaaaccgctaatgttcaaggacagttaccacaacctgcgcctctccctccatgacctcccccatgcccattggaggagcaagctgctggccaaataccaggagatccccttctatcacatttggagtggcagccagaaggccctccactgcactttcaccctggagaggcacagcttggcctccacagagctcacctgcaagatctgcgtgcggcaagtggaaggggagggccagatattccagctgcataccactctggcagagacacctgctggctccctggacactctctgctctgcccctggcagcactgtcaccacccagctgggaccttatgccttcaagatcccactgtccatccgccagaagatatgcaacagcctagatgcccccaactcacggggcaatgactggcggatgttagcacagaagctctctatggaccggtacctgaattactttgccaccaaagcgagccccacgggtgtgatcctggacctctgggaagctctgcagcaggacgatggggacctcaacagcctggcgagtgccttggaggagatgggcaagagtgagatgctggtggctgtggccaccgacggggactgctgagcctcctgggacagcgggctggcagggactggcaggaggcaggtgcagggaggcctggggcagcctcctgatggggatgtttggcctctgcttcctcccagttcacagccagagttgcctctcctcctcctcttccccaacccccagaccatgaccagccttagaaaatccatgtactctgttgttagagggcccagagttccttctccacccccgctctctctctcttggcctgagatctctgtgcaggaaccaagatggggctgaagcctctggaggcagttggttgggggcgggcaggcaggaggccctccctccacccccccaccctcagcccggcaacttctgggttccatgggttttagttccgttctcgttttcttcctccgttattgatttctcctttctccctaagcccccttctgcttccacgcccttttcctctttgaagagtcaagtacaattcagacaaactgctttctcctgtccaaaagcaaaaaggcaaaggaaagaaagaaagcttcagaccgctagtaaggctcaaagaagaagaaaaacaccaaaaccacaagggaaaagaaaaacccagtttcttaggaaacgcaaacgatttattatccagattatttggataagtcctttttaagaaaaaaaaaagaaaatgaaaaacaacacaaaaaaaaaaaaaaaa

SEQ ID NO: 11 - protein, 849 aa, homo sapiens
GTDSGSEVLPDSFPSAPAEPLPYFLQEPQDAYIVKNKPVELRCRAFPATQIYFKCNGEWVSQNDHVTQEGLDEATGLRVREVQIEVSRQQVEELFGLEDYWCQCVAWSSAGTTKSRRAYVRIAYLRKNFDQEPLGKEVPLDHEVLLQCRPPEGVPVAEVEWLKNEDVIDPTQDTNFLLTIDHNLIIRQARLSDTANYTCVAKNIVAKRRSTTATVIVYVNGGWSSWAEWSPCSNRCGRGWQKRTRTCTNPAPLNGGAFCEGQAFQKTACTTICPVDGAWTEWSKWSACSTECAHWRSRECMAPPPQNGGRDCSGTLLDSKNCTDGLCMQSNPQLLHPSVPPDLTASAGIYRGPVYALQDSTDKIPMTNSPLLDPLPSLKVKVYSSSTTGSGPGLADGADLLGVLPPGTYPSDFARDTHFLHLRSASLGSQQLLGLPRDPGSSVSGTFGCLGGRLSIPGTGVSLLVPNGAIPQGKFYEMYLLINKAESTLPLSEGTQTVLSPSVTCGPTGLLLCRPVILTMPHCAEVSARDWIFQLKTQAHQGHWEEVVTLDEETLNTPCYCQLEPRACHILLDQLGTYVFTGESYSRSAVKRLQLAVFAPALCTSLEYSLRVYCLEDTPVALKEVLELERTLGGYLVEEPKPLMFKDSYHNLRLSLHDLPHAHWRSKLLAKYQEIPFYHIWSGSQKALHCTFTLERHSLASTELTCKICVRQVEGEGQIFQLHTTLAETPAGSLDTLCSAPGSTVTTQLGPYAFKIPLSIRQKICNSLDAPNSRGNDWRMLAQKLSMDRYLNYFATKASPTGVILDLWEALQQDDGDLNSLASALEEMGKSEMLVAVATDGDCHHHHHH

(19) United States
(12) Patent Application Publication
(10) Pub. No.: US 2008/0038255 A1
(43) Pub. Date: Feb. 14, 2008

(54) Splice Variant of Unc5h2

(75) Inventors: Christine Power, Thoiry (FR); Melanie Yorke-Smith, Confignon (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Aplied Research Systems ARS Holding N.V., Curacao (NL)

(21) Appl. No.: 11/597,986

(22) PCT Filed: Jun. 3, 2005
(86) PCT No.: PCT/EP05/52573

§ 371 Date: Apr. 16, 2007

(30) Foreign Application Priority Data
Jun. 4, 2004 (EP) 04102511.5

(51) Int. Cl. A61K 31/70; A61K 39/395; A61P 43/00; C07H 21/04; C07K 16/00; C12N 15/00; C12N 5/00; C12P 21/04; G01N 33/53

(52) U.S. Cl. 424/133.1; 424/130.1; 435/320.1; 435/325; 435/346; 435/69.1; 435/7.1; 514/12; 514/44; 530/350; 536/23.5

(57) ABSTRACT

The present invention provides an UNC5H2 splice variant polypeptide, UNC5H2d, and the nucleic acid molecule encoding the same. The invention also provides selective binding agents, vectors, host cells, and methods for producing the UNC5H2d polypeptide. The invention further provides pharmaceutical compositions and methods for the diagnosis, treatment, amelioration, and/or prevention of diseases, disorders, and conditions associated with the UNC5H2d polypeptide.

### FIELD OF THE INVENTION

The present invention relates to an UNC5H2 splice variant polypeptide, UNC5H2d, and the nucleic acid molecule encoding the same. The invention also relates to selective binding agents, vectors, host cells, and methods for producing the UNC5H2d polypeptide. The invention further relates to pharmaceutical compositions and methods for the diagnosis, treatment, amelioration, and/or prevention of diseases, disorders, and conditions associated with the UNC5H2d polypeptide.

### BACKGROUND OF THE INVENTION

UNC-5 was first described in C. elegans as a gene required for axonal repulsion in netrin/UNC-6 responsive neurons (Williams M. E., 2003). Homologues of C. elegans UNC-5 comprise the Drosophila melanogaster UNC-5, the Mus musculus UNC-5, the Rattus norvegicus Unc5H2 and the Homo sapiens UNC-5 family (UNC5H1, 2, and 3). Homo sapiens gene UNC5H2 (also known as UNC5B, p53RDL1 or p53-regulated receptor for death and life) maps on chromosome 10 (cytogenetic band 10q22.1).

UNC5H2 Transcripts

According to Aceview's database (AceView shows the alignment of mRNAs and ESTs to the genome sequence, and the genes and (alternative) transcripts reconstructed from these alignments, using the Acembly program, http://www.ncbi.nlm.nih.gov/IEB/Research/Acembly/index.html) analysis, UNC5H2 produces, by alternative splicing, 3 types of transcripts, predicted to encode 3 distinct proteins. UNC5H2 variant a corresponds to the characterized SwissProt entry Q8IZJ1 and NCBI's entries NP_{—}734465, AAM95701 and AY126437 as described by Komatzuzaki et al., the b variant is a predicted protein that has yet not been characterized, and the Aceview's c variant probably corresponds to NCBI's entry BAC57998 as described by Tanikawa et al. and referred to as p53RDL1 (Aceview's entry is a truncated form of p53RDL1). UNC5H2c contains 17 exons spanning 2835 bp, UNC5H2a contains 16 exons and UNC5Hb contains 7 exons. Compared to UNC5H2c, UNC5H2a is missing exon 8, which is a short exon of only 11 amino acids and occurs at the boundary between the extracellular domain of the protein and the transmembrane-spanning domain. Compared to UNC5H2c, UNC5H2b is missing exons I to 5 and 12-17, which contain the Immunoglobulin domains, a Thrombospondin type 1 repeats domain and the death domain (see below), but do possess an additional new 3′ alternative exon. UNC5H2 contains 17 confirmed introns, 4 of which are alternative. Comparison to the genome sequence shows that 17 introns follow the consensual [gt-ag] rule. The protein family contains several identified domains, of which a Death domain, an Immunoglobulin/major histocompatibility complex motif, a Thrombospondin type I motif, and a ZU5 domain motif.

UNC5H2 Expression

According to Acembly, the protein is expressed at very high level. Acembly's annotation on the main supporting clones for UNC5H2 indicates that the tissues in which the mRNA was expressed include amygdala, spleen, osteoarthritic cartilage knee, neuroblastoma brain, synovial membrane and uterus. Genecards' annotation (Genecards is a database integrating information on human genes, http://bioinformatics.weizmann.ac.il/cards/) on UNC5H2 (Genecards' entry LOC219699) expression in normal human tissues based on the ESTs' quantification from various tissues in Unigene (http://www.ncbi.nlm.nih.gov/UniGene/clust.cgi?ORG=Hs) clusters include spleen, brain, prostate, kidney and lung (“UniGene is an experimental system for automatically partitioning GenBank sequences into a non-redundant set of gene-oriented clusters. Each UniGene cluster contains sequences that represent a unique gene, as well as related information such as the issue types in which the gene has been expressed and map location”). The expression information from Unigene indicates that UNC5H2 is expressed in the following tissues: neuroblastoma, adrenal cortex carcinoma, colon, large cell carcinoma, lung, placenta, osteoarthritic cartilage, spleen, bone, breast, head, neck, prostate, melanocytes, fetal heart, pregnant uterus, subchondral bone, lung focal fibrosis, chondrosarcoma and purified pancreatic islet.

Besides being highly expressed in specific neurons during the development of the nervous system in adult, UNC5H has also been detected in thyroid, kidney, ovary, uterus, stomach, colon, lung, spleen, bladder, breast tissues, heart, cartilage, hematopoietic and immune tissues (Komatsuzaki et al., 2002).

UNC5H2 Function

The UNC5H family, together with the Deleted in Colorectal Cancer (DCC) family, comprising DCC and neo1, are netrin-1 (ntn1) receptors. Netrin-1 is involved in short- and long-range guidance cue, directing neurons and their axons to targets during development of the nervous system (Mitchell K J, 1996). Its attractant or repellant bifunctional activity depends on the receptors expressed on the neurons and on the intracellular cAMP levels (Hong et al., 1999). The DCC family acts as a mediator in the attraction of netrin-1, whereas a netrin-1-dependent UNC5H-DCC complex is formed to mediate repulsion by G_{iα2} modulation.

Besides their role in axonal guidance, DCC and UNC5H2 are suggested to be tumor suppressors (Thiébault et al, 2003). In the absence of netrin-1, the UNC5H family drive cell death induction. This proapoptotic activity depends on the caspase cleavage of the receptors and the conserved death domain located in the C terminus of their intracellular domains (Tanikawa et al, 2003). UNC5H2 is a direct transcriptional target for the tumor suppressor p53 and mediates p53 proapoptotic activity (Tanikawa et al, 2003). Thiébault et al. suggested that UNC5H receptors might act as negative regulators of tumor growth probably by inducing cell apoptosis. It has been furthermore observed that UNC5H expression is lost or reduced in many cancers including ovary tumors, breast tumors, uterus tumors, colorectal tumors, stomach tumors, lung tumors and kidney tumors. Three mechanisms have been proposed that can regulate UNC5H expression:
1) Loss Of Heterozygosity (LOH); mutations in UNC5C as well as deletions of chromosome regions where the UNC5A, UNC5B and UNC5C locus have been mapped have been associated with various cancers.
2) Epigenetic processes; UNC5H expression has been shown to be regulated by histone deacetylase activity.
3) Loss of p53 activity may account for the loss/reduction in UNC5H expression.

Thiébault et al. further suggest that inhibition of UNC5H expression and/or inhibition of UNC5H proapoptotic activity would represent a selective process to permit the development of tumors and that the balance between netrin-1 and UNC5H expression might be crucial for tumor development control. In this perspective, netrin-1 also acts as a survival factor that controls cell fate, its presence gradually decreasing with aging epithelial cells. Thus, netrin-1 receptors DCC and UNC5H are normally switched off in an adequate environment and switched on in inadequate setting where netrin-1 is unavailable (e.g. during metastasis). Netrin-1 was shown to antagonize p53-induced UNC5B mediated apoptosis (Tanikawa et al., 2003).

### SUMMARY OF THE INVENTION

The present invention relates to a novel and distinct UNC5H2 alternative splicing variant, specifically designated UNC5H2 variant d (hereafter indicated as “UNC5H2d”). The UNC5H2d coding sequence is identical to the UNC5H2a coding sequence with the exception that the UNC5H2d coding sequence lacks exon 9 leading to the removal of the transmembrane domain. UNC5H2d lacks exons 8 and 9 compared with the longest splice variant UNC5H2c. Thus, UNC5H2d is a soluble secreted version of UNC5H2a, UNC5H2b, or UNC5H2c and likely functions as an antagonist of the integral UNC5H2 signaling pathway.

The invention provides for an isolated nucleic acid molecule comprising a nucleotide sequence selected from the group consisting of.
(a) the nucleotide sequence as set forth in any of SEQ ID NO: 1 or SEQ ID NO: 3;
(b) a nucleotide sequence encoding the polypeptide as set forth in any of SEQ ID NO: 2 or SEQ ID NO: 4 or SEQ ID NO: 11, or encoding a polypeptide which exhibits at least about 85% identity to the polypeptide as set forth in any of SEQ ID NO:2 or SEQ ID NO: 4 or SEQ ID NO: 11, wherein the encoded polypeptide has an activity of the polypeptide set forth in any of SEQ ID NO: 2 or SEQ ID NO: 4 or SEQ ID NO: 11, provided that such polypeptide sequence is not identical to any of SEQ ID NO: 5 or SEQ ID NO: 6 or SEQ ID NO: 7.
(c) a nucleotide sequence encoding the mature form of the polypeptide whose sequence is recited in SEQ ID NO: 2 (SEQ ID NO:4);
(d) a nucleotide sequence encoding the histidine tag form of the polypeptide whose sequence is recited in SEQ ID NO: 2 (SEQ ID NO: 11);
(e) a nucleotide sequence which hybridizes under stringent conditions with any of SEQ ID NO: 1 or SEQ ID NO:3, or exhibit at least about 85% identity over a stretch of at least about 30 nucleotides, with a nucleic acid consisting of any of SEQ ID NO: 1 or SEQ ID NO: 3, or be a complement of said DNA sequences, provided that such polynucleotide sequence is not identical to any of SEQ ID NO: 8 or SEQ ID NO: 9 or SEQ ID NO: 10.

The present invention also provides for an expression vector comprising the isolated nucleic acid molecule as set forth herein, recombinant host cells comprising the recombinant nucleic acid molecule as set forth herein, and a method of producing an UNC5H2d polypeptide comprising culturing the host cells and optionally isolating the polypeptide so produced.

A transgenic non-human animal comprising a nucleic acid molecule encoding an UNC5H2d polypeptide is also encompassed by the invention. The UNC5H2d nucleic acid molecule is introduced into the animal in a manner that allows expression and increased levels of an UNC5H2d polypeptide, which may include increased circulating levels. Alternatively, the UNC5H2d nucleic acid molecule is introduced into the animal in a manner that prevents expression of endogenous UNC5H2d polypeptide (i.e., generates a transgenic animal possessing an UNC5H2d polypeptide gene (knock-out)).

The transgenic non-human animal is preferably a mammal, and more preferably a rodent, such as a rat or a mouse.

Also provided are derivatives of the UNC5H2d polypeptide of the present invention, which include polypeptides comprising the amino acid sequence of any of SEQ ID NO: 2 or SEQ ID NO: 4 or SEQ ID NO: 11.

Additionally provided are selective binding agents such as antibodies and peptides capable of specifically binding the UNC5H2d polypeptide of the invention. Such antibodies and peptides may be agonistic or antagonistic.

Pharmaceutical compositions comprising the nucleotides, polypeptides, or selective binding agents of the invention and one or more pharmaceutically acceptable formulation agents are also encompassed by the invention. The pharmaceutical compositions are used to provide therapeutically effective amounts of the nucleotides or polypeptides of the present invention. The invention is also directed to methods of using the polypeptides, nucleic acid molecules, and selective binding agents.

The UNC5H2d polypeptide and nucleic acid molecule of the present invention may be used to treat, prevent, ameliorate, and/or detect diseases and disorders, including those recited herein.

The present invention also provides a method of assaying test molecules to identify a test molecule that binds to an UNC5H2d polypeptide. The method comprises contacting an UNC5H2d polypeptide with a test molecule to determine the extent of binding of the test molecule to the polypeptide. The method further comprises determining whether such test molecules are agonists or antagonists of an UNC5H2d polypeptide. The present invention further provides a method of testing the impact of molecules on the expression of UNC5H2d polypeptide or on the activity of UNC5H2d polypeptide.

Methods of regulating expression and modulating (i.e., increasing or decreasing) levels of an UNC5H2d polypeptide are also encompassed by the invention. One method comprises administering to an animal a nucleic acid molecule encoding an UNC5H2d polypeptide. In another method, a nucleic acid molecule comprising elements that regulate or modulate the expression of an UNC5H2d polypeptide may be administered. Examples of these methods include gene therapy, cell therapy, and anti-sense therapy as further described herein.

The UNC5H2d polypeptide can be used for identifying ligands thereof. Various forms of “expression cloning” have been used for cloning ligands for receptors (See, e.g., lo Davis et al., 1996, Cell, 87:1161-69). These and other UNC5H2d ligand cloning experiments are described in greater detail herein. Isolation of the UNC5H2d ligand(s) allows for the identification or development of novel agonists and/or antagonists of the UNC5H2d signaling pathway. Such agonists and antagonists include UNC5H2d ligand(s), anti-UNC5H2d ligand antibodies and derivatives thereof, small molecules, or antisense oligonucleotides, any of which can be used for potentially treating one or more diseases or disorders, including those recited herein.

### BRIEF DESCRIPTION OF THE FIGURES

FIG. 1 illustrates the amino acid alignment of UNC5H2a, UNC5H2b, UNC5H2c and UNC5H2d using the Multalin version 5.4.1 Copyright I.N.R.A. France 1989, 1991, 1994, 1996. Multiple sequence alignment with hierarchical clustering. F. CORPET, 1988, Nucl. Acids Res., 16 (22), 10881-10890.

Symbol comparison table used: blosum62 with Gap weight 12 and Gap length weight 2.

Consensus levels: high=90% low=50%

Consensus symbols used:

! is anyone of IV

$ is anyone of LM

% is anyone of FY

# is anyone of NDQEBZ

FIG. 2 represents the nucleotide sequence of UNC5H2c with translation.

FIG. 3 represents the nucleotide sequence with translation of UNC5H2d PCR product cloned using primers UNC5H2c-CP1 and UNC5H2c-CP2.

FIG. 4 illustrates the map of the plasmid pCR-XL-TOPO-UNC5H2d.

Molecule: pCR-XL-TOPO-UNC5H2d 6273 bps DNA Circular
Molecule Features:

| Type | Start | End | | Name | Description |
|---|---|---|---|---|---|
| REGION | 95 | 216 | | Plac | Lac promoter |
| MARKER | 205 | | | M13R | M13R primer |
| REGION | 337 | 3090 | C | Insert | Inserted UNC5H2d PCR product |
| GENE | 3032 | 426 | C | cds | UNC5H2d cds |
| MARKER | 3160 | | C | T7 | T7 promoter priming site |
| GENE | 3340 | 3642 | | ccdB | ccdB lethal gene ORF |
| GENE | 3991 | 4785 | | Kan | Kanamycin resistance ORF |
| GENE | 4992 | 5366 | | Zeo | Zeocin resistance ORF |
| REGION | 5434 | 6147 | | pUC | pUC origin |

FIG. 5 represents the nucleotide sequence with translation of UNC5H2d-6HIS PCR product amplified using primers UNC5H2d-DP1 and UNC5H2d-DP2.

FIG. 6 illustrates the Map of pEAK12M.

Molecule: peak12M, 6909 bps DNA Circular

| Type | Start | End | | Name | Description |
|---|---|---|---|---|---|
| REGION | 2 | 595 | | pmb-ori | |
| GENE | 659 | 1516 | | Amp | Ampicillin resistance ORF |
| GENE | 1690 | 2796 | | EF-1alpha | EF-1alpha promoter |
| REGION | 2846 | 2886 | | MCS | Polylinker |
| GENE | 3927 | 3331 | C | Pur | Puromycin resistance ORF |
| REGION | 4154 | 3931 | C | tK | tK promoter |
| GENE | 4649 | 4155 | C | Ori P | |
| GENE | 6701 | 4649 | C | EBNA-1 | EBNA-1 ORF |
| REGION | 6702 | 6901 | | sv40 | sv40 promoter |

FIG. 7 illustrates the Map of pcDNA3.1.

Molecule: pcDNA3.1+, 5428 bps DNA Circular

| Type | Start | End | Name | Description |
|---|---|---|---|---|
| MARKER | 209 | | CMV | CMV promoter |
| MARKER | 863 | | T7 | T7 promoter |
| REGION | 1021 | 1235 | BGH polyA | |
| MARKER | 1021 | | BGHrev | BGH reverse priming site |
| REGION | 1298 | 1711 | f1 | f1 origin |
| REGION | 1776 | 2101 | SV40 prom | SV40 promoter |
| GENE | 2137 | 2931 | Neo | Neomycin resistance ORF |
| REGION | 2947 | 3186 | SV40 polyA | |
| REGION | 3618 | 4291 | ColE1 | ColE1 origin |
| GENE | 4436 | 5297 | Amp | Ampicillin resistance ORF |

FIG. 8 illustrates the Map of pEAK12M-UNC5H2d-6HIS.

Molecule: pEAK12M-UNC5H2d-6HIS, 9543 bps DNA Circular

| Type | Start | End | | Name | Description |
|---|---|---|---|---|---|
| REGION | 2 | 595 | | pmb-ori | |
| GENE | 659 | 1516 | | Amp | Ampicillin resistance ORF |
| GENE | 1690 | 2796 | | EF-1alpha | |
| MARKER | 2703 | | | pEAK12-F | pEAK12-F priming site |
| MARKER | 2846 | | | UNC5H2d-DP1 | UNC5H2d-DP1 priming site |
| GENE | 2858 | 5464 | | cds | UNC5H2d cds |
| MARKER | 3407 | | | UNC5H2d-SP1 | UNC5H2d-SP1 priming site |
| MARKER | 4143 | | | UNC5H2d-SP2 | UNC5H2d-SP2 priming site |
| MARKER | 4500 | | | UNC5H2d-SP3 | UNC5H2d-SP3 priming site |
| MARKER | 5465 | | | 6HIS | 6HIS tag |
| MARKER | 5483 | | | stop | Stop codon |
| MARKER | 5491 | | C | UNC5H2d-DP2 | UNC5H2d-DP2 priming site |
| MARKER | 5631 | | C | pEAK12-R | pEAK12-R priming site |
| GENE | 6561 | 5965 | C | PUR | Puromycin resistance ORF |
| REGION | 6788 | 6565 | C | tK | tK promoter |
| GENE | 7283 | 6789 | C | Ori P | |
| GENE | 9335 | 7283 | C | EBNA-1 | |
| REGION | 9336 | 9535 | | sv40 | |

FIG. 9 illustrates the Map of pcDNA3.1-UNC5H2d-6HIS.

Molecule: pcDNA3.1-UNC5H2d-6HIS, 8027 bps DNA Circular

| Type | Start | End | | Name | Description |
|---|---|---|---|---|---|
| MARKER | 209 | | | CMV | CMV promoter |
| MARKER | 863 | | | T7 | T7 promoter |
| MARKER | 911 | | | UNC5H2d-D1 | UNC5H2d-D1 priming site |
| GENE | 923 | 3529 | | cds | UNC5H2d cds |
| MARKER | 1472 | | | UNC5H2d-SP1 | UNC5H2d-SP1 priming site |
| MARKER | 2208 | | | UNC5H2d-SP2 | UNC5H2d-SP2 priming site |
| MARKER | 2565 | | | UNC5H2d-SP3 | UNC5H2d-SP3 priming site |
| MARKER | 3530 | | | 6HIS | 6HIS tag |
| MARKER | 3548 | | | stop | Stop codon |
| MARKER | 3556 | | C | UNC5H2d-DP2 | UNC5H2d-DP2 priming site |
| MARKER | 3620 | | | BGHrev | BGH reverse priming site |
| MARKER | 3897 | | | f1 | f1 origin |
| MARKER | 4375 | | | SV40 prom | SV40 promoter |
| GENE | 4736 | 5530 | | Neo | Neomycin resistance ORF |
| MARKER | 6216 | | | pUC | pUC origin |
| MARKER | 6216 | | | ColE1 | ColE1 origin |
| GENE | 7896 | 7035 | C | Amp | Ampicillin resistance ORF |

FIG. 10 reports the Pfam domain alignment of UNC5H2d and UNC5H2a.

FIG. 11 reports the SMART Domains alignment of UNC5H2c, UNC5H2a, UNC5H2d and UNC5Hb.

### DETAILED DESCRIPTION OF THE INVENTION

The section headings used herein are for organizational purposes only and are not to be construed as limiting the subject matter described. All references cited in this application are expressly incorporated by reference herein.

The present invention refers to a new splice variant of transmembrane UNC5H2 (UNC5B), namely UNC5H2d, and its uses. Thus, it will be appreciated that UNC5H2d is a secreted soluble form of integral UNC5H2a, UNC5H2b or UNC5H2c. UNC5H2d is lacking the transmembrane region (see FIGS. 10 and 11). In this regard UNC5H2d may act as an antagonist of the UNC5H2 receptors or ligand(s). UNC5H2d can also be used as a target for antagonistic and agonistic molecules, including, but not limited to, antibodies, fusion polypeptides, carbohydrates, polynucleotides (such as antisense oligonucleotides), or small molecular weight organic molecules. For example, an antagonist specific for UNC5H2d would inhibit the antagonistic activity of UNC5H2d, thus enhancing the activity of UNC5H2d ligand(s) and/or enhancing signaling through UNC5H2 receptors. Conversely an agonist specific for UNC5H2 receptors would enhance the antagonistic activity of UNC5H2d, thus diminishing the activity of UNC5H2 receptor ligand(s) and/or diminishing signaling through UNC5H2 receptors.

Definitions

The terms “UNC5H2d gene” or “UNC5H2d nucleic acid molecule” or “UNC5H2d polynucleotide”, as used interchangeably herein, refer to an isolated nucleic acid molecule comprising a nucleotide sequence selected from the group consisting of:
(a) the nucleotide sequence as set forth in any of SEQ ID NO: 1 or SEQ ID NO: 3;
(b) a nucleotide sequence encoding the polypeptide as set forth in any of SEQ ID NO: 2 or SEQ ID NO: 4 or SEQ ID NO: 11, or encoding a polypeptide which exhibits at least about 85% identity to the polypeptide as set forth in any of SEQ ID NO:2 or SEQ ID NO: 4 or SEQ ID NO: 11, wherein the encoded polypeptide has a biological activity activity of the polypeptide set forth in any of SEQ ID NO: 2 or SEQ ID NO: 4 or SEQ ID NO: 11, provided that such polypeptide sequence is not identical to any of SEQ ID NO: 5 or SEQ ID NO: 6 or SEQ ID NO: 7.
(c) a nucleotide sequence encoding the mature form of the polypeptide whose sequence is recited in SEQ ID NO: 2 (SEQ ID NO:4);
(d) a nucleotide sequence encoding the histidine tag form of the polypeptide whose sequence is recited in SEQ ID NO: 2 (SEQ ID NO: 11);
(e) a nucleotide sequence which hybridizes under stringent conditions with any of SEQ ID NO: 1 or SEQ ID NO:3, or exhibit at least about 85% identity over a stretch of at least about 30 nucleotides, with a nucleic acid consisting of any of SEQ ID NO: 1 or SEQ ID NO: 3, or be a complement of said DNA sequences, provided that such polynucleotide sequence is not identical to any of SEQ ID NO: 8 or SEQ ID NO: 9 or SEQ ID NO: 10.

An “UNC5H2d polypeptide” as used herein refers to a polypeptide encoded by an UNC5H2d gene.

The polypeptide having the sequence recited in SEQ ID NO:2 is an UNC5H2d full length polypeptide. The polypeptide having the sequence recited in SEQ ID NO:4 is a mature UNC5H2d polypeptide. The polypeptide having the sequence recited in SEQ ID NO:11 is a mature his tagged UNC5H2d polypeptide. Thus, in one embodiment, an UNC5H2d polypeptide is a polypeptide comprising the amino acid sequence of any of SEQ ID NO: 2 or SEQ ID NO: 4 or SEQ ID NO: 11.

The polypeptide having the sequence recited in SEQ ID NO: 5 is an UNC5H2a polypeptide. The polypeptide having the sequence recited in SEQ ID NO: 6 is an UNC5H2b polypeptide. The polypeptide having the sequence recited in SEQ ID NO: 7 is an UNC5H2c polypeptide.

The nucleic acid having the sequence recited in SEQ ID NO:1 encodes the UNC5H2d full length polypeptide (SEQ ID NO:2). The nucleic acid having the sequence recited in SEQ ID NO:3 encodes the mature UNC5H2d polypeptide (SEQ ID NO:4). The nucleic acid sequence as recited in SEQ ID NO:8 encodes the UNC5H2a polypeptide (SEQ ID NO: 5). The nucleic acid sequence as recited in SEQ ID NO:9 encodes the UNC5H2b polypeptide (SEQ ID NO: 6). The nucleic acid sequence as recited in SEQ ID NO:10 encodes the UNC5H2c polypeptide (SEQ ID NO: 7).

The term “isolated nucleic acid molecule” refers to a nucleic acid molecule of the invention that (1) has been separated from at least about 50 percent of proteins, lipids, carbohydrates, or other materials with which it is naturally found when total nucleic add is isolated from the source cells, (2) is not linked to all or a portion of a polynucleotide to which the “isolated nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use.

The term “nucleic acid sequence” or “nucleic acid molecule” refers to a DNA or RNA sequence. The term encompasses molecules formed from any of the known base analogs of DNA and RNA such as, but not limited to 4-acetylcytosine, 8-hydroxy-N6-methyladenosine, aziridinyl-cytosine, pseudoisocytosine, 5 (carboxyhydroxylmetliyl)uracil, 5-fluorouracil, 5-bromouracil, 5 carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2 methyladenine, 2-methylguanine, 3-methylcytosine, 5-methylcytosine, N6 methyladenine, 7-methylguanine, 5-methylaminomethyluracil, 5-methoxyamino methyl-2-thiouracil, beta-D-mannosylqueosine, 5′-methoxycarbonyl-methyluracil, 5 methoxyuracil, 2-methylthio-N6-isopentenyladenine, uracil-5-oxyacetic acid methylester, uracil-5-oxyacetic acid, oxybutoxosine, pseudouracil, queosine, 2 thiocytosine, 5-methyl-2-thiouracil, 2-thiouracil, 4-thiouracil, 5-methyluracil, N uracil-5-oxyacetic acid methylester, uracil-5-oxyacetic acid, pseudouracil, queosine, 2-thiocytosine, and 2,6-diaminopurine.

The present invention also refers to recombinant DNA molecules, which hybridize with the DNA sequence coding for UNC5H2d or fragments thereof. The gene can contain, or not, the natural introns and can be obtained for example by extraction from appropriate cells and purification with known methods.

Appropriate preparations of DNA, as human genomic DNA, are cut in the appropriate way, preferably with restriction enzymes, and the so obtained fragments are introduced in appropriate recombinant vectors in order to form a DNA library. Such vectors can be selected with synthetic oligonucleotide probes in order to identify a sequence encoding the UNC5H2d according to the invention.

On the other hand, the corresponding mRNA can be isolated from the cells expressing the UNC5H2d and used to produce the complementary DNA (cDNA) with known methods. This cDNA after having been converted into double stranded cDNA, can be introduced in an appropriate vector which can afterwards be used for transforming an appropriated host cell.

The resulting cultures are then selected with an appropriate probe in order to obtain the cDNA encoding the targeted sequences.

Once the desired clone is isolated, the cDNA can be manipulated essentially in the same way as the genomic DNA. The cDNA does not contain introns.

Because of the degeneracy of the genetic code, various codons can be used for encoding a specific amino acid, so that one or more oligonucleotides can be produced, each of them being able to encode fragments of UNC5H2d. However only one member of this pool possesses the nucleotide sequence identical to that of the gene. Its presence in the pool and its capacity of hybridizing with the DNA also in the presence of other members of the pool makes it possible to use the group of non fractioned oligonucleotides in the same way as a single oligonucleotide could be used for cloning the gene encoding the targeted peptide.

Alternatively, a single oligonucleotide containing the sequence which is theoretically the most probable of being able to encode the genic fragments of UNC5H2d (according to that described in the “rules for the use of codons” in Lathe R, et al. J. Molec. Biol. 183:1-12 (1985)) allows the identification the complementary DNA encoding UNC5H2d or a fragment thereof.

The processes for hybridizing the nucleic acids are known and described (for example in Maniatis T. et al. Molecular Cloning: A laboratory manual, Cold Spring Harbor Press, Cold Spring Harbor, N.Y., 1982). Through the hybridization using said probe or group of nucleotide probes it is possible to identify in a genomic or cDNA gene library the DNA sequences capable of such hybridization which are thereafter analyzed to confirm that they encode the polypeptide according to the invention. The oligonucleotide, which contains such complementary sequence can be synthesized and used as probe to identify and isolate the gene of the polypeptide according to the invention (Maniatis T. et al. ibid.). Once the appropriate oligonucleotide specific for the UNC5H2d is selected using the above said method, it is possible to synthesize and hybridize it with a DNA, or preferably with a cDNA derived from cells capable of expressing the wanted gene preferably after the source of cDNA was enriched of wanted sequences, for example by extraction of the RNA from cells producing high levels of the wanted gene and conversion of the RNA into the corresponding cDNA using the enzyme reverse transcriptase.

Alternatively, the suitable oligonucleotides specific for UNC5H2d can be synthesised and used as primers for the amplification of UNC5H2d cDNA fragments by RACE-PCR.

The term “substantially purified” refers to molecules, either nucleic or amino acid sequences, that are removed from their natural environment, isolated or separated, and are at least 60% free, preferably 75% free, and most preferably, 90% free from other components with which they are naturally associated.

The term “hybridization” as used herein shall include any process by which a strand of nucleic acid joins with complementary strand through a base pairing. “Amplification” is defined as the production of additional copies of a nucleic acid sequence and is generally carried out using polymerase chain reaction technologies well known in the art.

“Stringency” typically occurs in a range from about Tm-5° C. (5° C. below the melting temperature of the probe) to about 20° C. to 25° C. below Tm.

The term “stringent conditions” refers to hybridization and subsequent washing conditions, which those of ordinary skill in the art conventionally refer to as “stringent”.

As used herein, stringency conditions are a function of the temperature used in the hybridization experiment, the molarity of the monovalent cations and the percentage of formamide in the hybridization solution. To determine the degree of stringency involved with any given set of conditions, one first uses the equation of Meinkoth et al. (1984) for determining the stability of hybrids of 100% identity expressed as melting temperature Tm of the DNA-DNA hybrid:
Tm=81.5 C+16.6 (Log M)+0.41 (% GC)−0.61 (% form)−500/L
where M is the molarity of monovalent cations, % GC is the percentage of G and C nucleotides in the DNA, % form is the percentage of formamide in the hybridization solution, and L is the length of the hybrid in base pairs. For each 1° C. that the Tm is reduced from that calculated for a 100% identity hybrid, the amount of mismatch permitted is increased by about 1%. Thus, if the Tm used for any given hybridization experiment at the specified salt and formamide concentrations is 10° C. below the Tm calculated for a 100% hybrid according to the equation of Meinkoth, hybridization will occur even if there is up to about 10% mismatch.

As used herein, “highly stringent conditions” are those which provide a Tm which is not more than 10° C. below the Tm that would exist for a perfect duplex with the target sequence, either as calculated by the above formula or as actually measured. “Moderately stringent conditions” are those, which provide a Tm, which is not more than 20° C. below the Tm that would exist for a perfect duplex with the target sequence, either as calculated by the above formula or as actually measured. Without limitation, examples of highly stringent (5-10° C. below the calculated or measured Tm of the hybrid) and moderately stringent (15-20° C. below the calculated or measured Tm of the hybrid) conditions use a wash solution of 2×SSC (standard saline citrate) and 0.5% SDS (sodium dodecyl sulfate) at the appropriate temperature below the calculated Tm of the hybrid. The ultimate stringency of the conditions is primarily due to the washing conditions, particularly if the hybridization conditions used are those, which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE (standard saline-phosphate-EDTA)), 5×Denhardt's reagent, 0.5% SDS, 100 microg/ml denatured, fragmented salmon sperm DNA at a temperature approximately 20 to 25° C. below the Tm. If mixed probes are used, it is preferable to use tetramethyl ammonium chloride (TMAC) instead of SSC.

The term “vector” is used to refer to any molecule (e.g., nucleic acid, plasmid, or virus) used to transfer coding information to a host cell.

The term “expression vector” refers to a vector that is suitable for transformation of a host cell and contains nucleic acid sequences that direct and/or control the expression of inserted heterologous nucleic acid sequences. Expression includes, but is not limited to, processes such as transcription, translation, and RNA splicing, if introns are present.

The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The term “naturally occurring or “native” when used in connection with biological materials such as nucleic acid molecules, polypeptides, host cells, and the like, refers to materials which are found in nature and are not manipulated by man. Similarly, “non-naturally occurring or “non-native” as used herein refers to a material that is not found in nature or that has been structurally modified or synthesized by man.

The terms “effective amount” and “therapeutically effective amount” each refer to the amount of an UNC5H2d polypeptide or UNC5H2d nucleic add molecule used to support an observable level of one or more biological activities of the UNC5H2d polypeptide as set forth herein.

The term “pharmaceutically acceptable carrier” or “physiologically acceptable carrier” as used herein refers to one or more formulation materials suitable for accomplishing or enhancing the delivery of the UNC5H2d polypeptide, UNC5H2d nucleic acid molecule, or UNC5H2d selective binding agent as a pharmaceutical composition.

The term “antigen” refers to a molecule or a portion of a molecule capable of being bound by a selective binding agent, such as an antibody, and additionally capable of being used in an animal to produce antibodies capable of binding to an epitome of that antigen. An antigen may have one or more epitomes.

The term “selective binding agent” refers to a molecule or molecules having specificity for an UNC5H2d polypeptide. As used herein, the terms “specific” and “specificity” preferably refer to the ability of the selective binding agents to bind to a human UNC5H2d polypeptide and preferentially not to a human non-UNC5H2d polypeptide. It will be appreciated, however, that the selective binding agents may also bind orthologs of the polypeptide as set forth in any of SEQ ID NO: 2 or SEQ ID NO: 4 or SEQ ID NO: 11.

Nucleic Acid Molecules

The nucleic acid molecules encoding a polypeptide comprising the amino acid sequence of an UNC5H2d polypeptide can readily be obtained in a variety of ways including, without limitation, chemical synthesis, cDNA or genomic library screening, expression library screening, and/or PCR amplification of cDNA.

Recombinant DNA methods used herein are generally those set forth in Sambrook et al., Molecular Cloning: A Laboratory Manual (Cold Spring Harbor Laboratory Press, 1989). The invention provides for nucleic acid molecules as described herein and methods for obtaining such molecules.

Where a gene encoding the amino acid sequence of an UNC5H2d polypeptide has been identified from one species, all or a portion of that gene may be used as a probe to identify orthologs or related genes from the same species. The probes or primers may be used to screen cDNA libraries from various tissue sources believed to express the UNC5H2d polypeptide. In addition, part or all of a nucleic acid molecule having the sequence as set forth in any SEQ ID NO: 1 or SEQ ID NO: 3 may be used to screen a genomic library to identify and isolate a gene encoding the amino acid sequence of an UNC5H2d polypeptide. Typically, conditions of moderate or high stringency will be employed for screening to minimize the number of false positives obtained from the screening.

Nucleic acid molecules encoding the amino acid sequence of the UNC5H2d polypeptide may also be identified by expression cloning which employs the detection of positive clones based upon a property of the expressed protein. Typically, nucleic acid libraries are screened by the binding an antibody or other binding partner (e.g., receptor or ligand) to cloned proteins that are expressed and displayed on a host cell surface. The antibody or binding partner is modified with a detectable label to identify those cells expressing the desired clone.

Recombinant expression techniques conducted in accordance with the descriptions set forth below may be followed to produce these polynucleotides and to express the encoded polypeptides. For example, by inserting a nucleic acid sequence that encodes the amino add sequence of an UNC5H2d polypeptide into an appropriate vector, one skilled in the art can readily produce large quantities of the desired nucleotide sequence. The sequences can then be used to generate detection probes or amplification primers.

Alternatively, a polynucleotide encoding the amino acid sequence of an UNC5H2d polypeptide can be inserted into an expression vector. By introducing the expression vector into an appropriate host, the encoded UNC5H2d polypeptide may be produced in large amounts.

Another method for obtaining a suitable nucleic acid sequence is the polymerase chain reaction (PCR). In this method, cDNA is prepared from poly (A)+RNA or total RNA using the enzyme reverse transcriptase. Two primers, typically complementary to two separate regions of cDNA encoding the amino acid sequence of an UNC5H2d polypeptide, are then added to the cDNA along with a polymerase such as Taq polymerase, and the polymerase amplifies the cDNA region between the two primers.

Another means of preparing a nucleic add molecule encoding the amino acid sequence of an UNC5H2d polypeptide is chemical synthesis using methods well known to the skilled artisan such as those described by Engels et al., 1989, Angew. Chem. Int. Ed. 28:716-34. These methods include, inter alia, the phosphotriester, phosphoramidite, and H-phosphonate methods for nucleic acid synthesis. A preferred is method for such chemical synthesis is polymer-supported synthesis using standard phosphoramidite chemistry. Typically, the DNA encoding the amino acid sequence of an UNC5H2d polypeptide will be several hundred nucleotides in length. Nucleic acids larger than about 100 nucleotides can be synthesized as several fragments using these methods. The fragments can then be ligated together to form the full-length nucleotide sequence of an UNC5H2d gene. Usually, the DNA fragment encoding the amino-terminus of the polypeptide will have an ATG, which encodes a methionine residue. This methionine may or may not be present on the mature form of the UNC5H2d polypeptide, depending on whether the polypeptide produced in the host cell is designed to be secreted from that cell. Other methods known to the skilled artisan may be used as well.

In certain embodiments, nucleic acid variants contain codons which have been altered for optimal expression of an UNC5H2d polypeptide in a given host cell. Particular codon alterations will depend upon the UNC5H2d polypeptide and host cell selected for expression. Such ‘codon optimization’ can be carried out by a variety of methods, for example, by selecting codons which are preferred for use in highly expressed genes in a given host cell. Computer algorithms, which incorporate codon frequency tables such as “Eco-high.Cod” for codon preference of highly expressed bacterial genes, may be used and are provided by the University of Wisconsin Package Version 9.0 (Genetics Computer Group, Madison, Wis.). Other useful codon frequency tables include “C. elegans—high.cod,” “C. elegans—low.cod,” “Drosophila—high.cod, “Human—high.cod”, “Maize—high.cod,” and “Yeast—high.cod.”

In some cases, it may be desirable to prepare nucleic acid molecules encoding UNC5H2d polypeptide variants. Nucleic acid molecules encoding variants may be produced using site directed mutagenesis, PCR amplification, or other appropriate methods, where the primer(s) have the desired point mutations (see e.g. Sambrook et al., for descriptions of mutagenesis techniques). Chemical synthesis using methods described by Engels et al., supra, may also be used to prepare such variants. Other methods known to the skilled artisan may be used as well.

Vectors and Host Cells

A nucleic acid molecule encoding the amino acid sequence of an UNC5H2d polypeptide is inserted into an appropriate expression vector using standard ligation techniques. The vector is typically selected to be functional in the particular host cell employed (i.e., the vector is compatible with the host cell machinery such that amplification of the gene and/or expression of the gene can occur). A nucleic acid molecule encoding the amino acid sequence of an UNC5H2d polypeptide may be amplified/expressed in prokaryotic, yeast, insect (baculovirus systems) and/or eukaryotic host cells. Selection of the host cell will depend in part on whether an UNC5H2d polypeptide is to be post-translationally modified (e.g., glycosylated and/or phosphorylated). If so, yeast, insect, or mammalian host cells are preferable.

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the UNC5H2d polypeptide coding sequence; the oligonucleotide sequence encodes polyHis (such as hexaHis), or another “tag” such as FLAG, HA (hemaglutinin influenza virus), or myc for which commercially available antibodies exist. This tag is typically fused to the polypeptide upon expression of the polypeptide, and can serve as a means for affinity purification of the UNC5H2d polypeptide from the host cell. Affinity purification can be accomplished, for example, by column chromatography using antibodies against the tag as an affinity matrix. Optionally, the tag can subsequently be removed from the purified UNC5H2d polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), or synthetic, or the flanking sequences may be native sequences that normally function to regulate UNC5H2d polypeptide expression. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein—other than the UNC5H2d gene flanking sequences—will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Where all or only a portion of the flanking sequence is known, it may be obtained using PCR and/or by screening a genomic library with a suitable oligonucleotide and/or flanking sequence fragment from the same or another species.

Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. Amplification of the vector to a certain copy number can, in some cases, be important for the optimal expression of an UNC5H2d polypeptide. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria and various origins (e.g., SV40, polyoma, is adenovirus, vesicular stomatitis virus (VSV), or papillomaviruses such as HPV or BPV) are useful for cloning vectors in mammalian cells. Generally, the origin of replication component is not needed for mammalian expression vectors (for example, the SV40 origin is often used only because it contains the early promoter).

A transcription termination sequence is typically located 3′ of the end of a polypeptide-coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis such as those described herein.

A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells. Other selection genes may be used to amplify the gene that will be expressed.

Amplification is the process wherein genes that are in greater demand for the production of a protein critical for growth are reiterated in tandem within the chromosomes of successive generations of recombinant cells. Examples of suitable selectable markers for mammalian cells include dihydrofolate reductase (DHFR) and thymidine kinase. The mammalian cell transformants are placed under selection pressure wherein only the transformants are uniquely adapted to survive by virtue of the selection gene present in the vector. Selection pressure is imposed by culturing the transformed cells under conditions in which the concentration of selection agent in the medium is successively changed, thereby leading to the amplification of both the selection gene and the DNA that encodes an UNC5H2d polypeptide. As a result, increased quantities of UNC5H2d polypeptide are synthesized from the amplified DNA.

A ribosome-binding site is usually necessary for translation initiation of mRNA and is characterized by a Shine-Dalgamo sequence (prokaryotes) or a Kozak sequence (eukaryotes). The element is typically located 3′ to the promoter and 5′ to the coding sequence of an UNC5H2d polypeptide to be expressed. The Shine-Dalgamo sequence is varied but is typically a polypurine (i.e., having a high A-G content). Many Shine-Dalgamo sequences have been identified, each of which can be readily synthesized using methods set forth herein and used in a prokaryotic vector.

A leader, or signal, sequence may be used to direct an UNC5H2d polypeptide out of the host cell. Typically, a nucleotide sequence encoding the signal sequence is positioned in the coding region of an UNC5H2d nucleic acid molecule, or directly at the 5′ end of an UNC5H2d polypeptide-coding region. Many signal sequences have been identified, and any of those that are functional in the selected host cell may be used in conjunction with an UNC5H2d nucleic add molecule. Therefore, a signal sequence may be homologous (naturally occurring) or heterologous to the UNC5H2d nucleic acid molecule. Additionally, a signal sequence may be chemically synthesized using methods described herein. In most cases, the secretion of an UNC5H2d polypeptide from the host cell via the presence of a signal peptide will result in the removal of the signal peptide from the secreted UNC5H2d polypeptide. The signal sequence may be a component of the vector, or it may be a part of an UNC5H2d nucleic acid molecule that is inserted into the vector.

Included within the scope of this invention is the use of either a nucleotide sequence encoding a native UNC5H2d polypeptide signal sequence joined to an UNC5H2d polypeptide coding region or a nucleotide sequence encoding a heterologous signal sequence joined to an UNC5H2d polypeptide coding region. The heterologous signal sequence selected should be one that is recognized and processed, i.e., cleaved by a signal peptidase, by the host cell. For prokaryotic host cells that do not recognize and process the native UNC5H2d polypeptide signal sequence, the signal sequence is substituted by a prokaryotic signal sequence selected, for example, from the group of the alkaline phosphatase, penicillinase, or heat-stable enterotoxin 11 leaders. For yeast secretion, the native UNC5H2d polypeptide signal sequence may be substituted by the yeast invertase, alpha factor, or acid phosphatase leaders. In mammalian cell expression the native signal sequence is satisfactory, although other mammalian signal sequences may be suitable.

In some cases, such as where glycosylation is desired in a eukaryotic host cell expression system, one may manipulate the various presequences to improve glycosylation or yield. For example, one may alter the peptidase cleavage site of a particular signal peptide, or add pro-sequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired UNC5H2d polypeptide, if the enzyme cuts at such area within the mature polypeptide.

In many cases, transcription of a nucleic acid molecule is increased by the presence of one or more introns in the vector; this is particularly true where a polypeptide is produced in eukaryotic host cells, especially mammalian host cells. The introns used may be naturally occurring within the UNC5H2 gene especially where the gene used is a full-length genomic sequence or a fragment thereof. Where the intron is not naturally occurring within the gene (as for most cDNAs), the intron may be obtained from another source. The position of the intron with respect to flanking sequences and the UNC5H2 gene is generally important, as the intron must be transcribed to be effective. Thus, when an UNC5H2d cDNA molecule is being transcribed, the preferred position for the intron is 3′ to the transcription start site and 5′ to the poly-A transcription termination sequence. Preferably, the intron or introns will be located on one side or the other (i.e., 5′ or Y) of the cDNA such that it does not interrupt the coding sequence. Any intron from any source, including Viral, prokaryotic and eukaryotic (plant or animal) organisms, may be used to practice this invention, provided that it is compatible with the host cell into which it is inserted. Also included herein are synthetic introns. Optionally, more than one intron may be used in the vector.

The expression and cloning vectors of the present invention will typically contain a promoter that is recognized by the host organism and operably linked to the molecule encoding the UNC5H2d polypeptide. Promoters are untranscribed sequences located upstream (i.e., Y) to the start codon of a structural gene (generally within about 100 to 1000 bp) that control the transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding the UNC5H2d polypeptide by removing the promoter from the source DNA by restriction enzyme digestion and inserting the desired promoter sequence into the vector. The native UNC5H2d promoter sequence may be used to direct amplification and/or expression of an UNC5H2d nucleic acid molecule. A heterologous promoter is preferred, however, if it pen-nits greater transcription and higher yields of the expressed protein as compared to the native promoter, and if it is compatible with the host cell system that has been selected for use.

Promoters suitable for use with prokaryotic hosts include the beta-lactamase and lactose promoter systems; alkaline phosphatase; a tryptophan (trp) promoter system; and hybrid promoters such as the tac promoter. Other known bacterial promoters are also suitable. Their sequences have been published, thereby enabling one skilled in the art to ligate them to the desired DNA sequence, using linkers or adapters as needed to supply any useful restriction sites.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest in controlling the UNC5H2d gene expression include, but are not limited to: the SV40 early promoter region; the CW promoter; the promoter contained in the 3′ long terminal repeat of Rous sarcoma virus; the herpes thymidine kinase promoter; the regulatory sequences of the metallothionine gene; prokaryotic expression vectors such as the beta-lactamase promoter; or the tae promoter. Also of interest are the following animal transcriptional control regions, is which exhibit tissue specificity and have been utilized in transgenic animals: the elastase I gene control region which is active in pancreatic cells; the insulin gene control region which is active in pancreatic beta cells; the immunoglobulin gene control region which is active in lymphoid cells; the mouse mammary tumor virus control region which is active in testicular, breast, lymphoid and mast cells; the albumin gene control region which is active in liver; the alpha-feto-protein gene control region which is active in liver; the alpha 1-antitrypsin gene control region which is active in the liver; the beta-globin gene control region which is active in myeloid cells; the myelin basic protein gene control region which is active in oligodendrocyte cells in the brain; the myosin light chain-2 gene control region which is active in skeletal muscle; and the gonadotropic releasing hormone gene control region which is active in the hypothalamus.

An enhancer sequence may be inserted into the vector to increase the transcription of a DNA encoding an UNC5H2d polypeptide of the present invention by higher eukaryotes. Enhancers are cis-acting elements of DNA, usually about 10-300 bp in length, that act on the promoter to increase transcription. Enhancers are relatively orientation and position independent. They have been found 5′ and 3′ to the transcription unit. Several enhancer sequences available from mammalian genes are known (e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for the activation of eukaryotic promoters. While an enhancer may be spliced into the vector at a position 5′ or 3′ to an UNC5H2d nucleic acid molecule, it is typically located at a site 5′ from the promoter.

Expression vectors of the invention may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

Preferred vectors for practicing this invention are those that are compatible with bacterial, insect, and mammalian host cells. Such vectors include, inter alia, pCRII, pCR3, and pCDNA3.1 (Invitrogen, San Diego, Calif.), pBSII (Stratagene, La Jolla, Calif.), pET 15 (Novagen, Madison, Wis.), pGEX (Pharmacia Biotech, Piscataway, N.J.), pEGFP-N2 (Clontech, Palo Alto, Calif.), pETL (BlueBaell, Invitrogen), pDSRalpha (PCT Pub. No. WO 90/14363) and pFastBacDual (Gibco-BRL, Grand Island, N.Y.).

Additional suitable vectors include, but are not limited to, cosmids, plasmids, or modified viruses, but it will be appreciated that the vector system must be compatible with the selected host cell. Such vectors include, but are not limited to plasmids such as Bluescript plasmid derivatives (a high copy number CoIEI-based phagemid; Stratagene Cloning Systems, La Jolla Calif.), PCR cloning plasmids designed for cloning Taq-amplified PCR products (e.g., TOPO™ TA Cloning “Kit and PCR2.1” plasmid derivatives; Invitrogen), and mammalian, yeast or virus vectors such as a baculovirus expression system (pBacPAK plasmid derivatives; Clontech).

After the vector has been constructed and a nucleic acid molecule encoding an UNC5H2d polypeptide has been inserted into the proper site of the vector, the completed vector may be inserted into a suitable host cell for amplification and/or polypeptide expression. The transformation of an expression vector for an UNC5H2d polypeptide into a selected host cell may be accomplished by well-known methods including methods such as transfection, infection, calcium chloride, electroporation, microinjection, lipofection, DEAE-dextran method, or other known techniques. The method selected will in part be a function of the type of host cell to be used. These methods and other suitable methods are well known to the skilled artisan, and are set forth, for example, in Sambrook et al., supra.

Host cells may be prokaryotic host cells (such as E. coli) or eukaryotic host cells (such as a yeast, insect, or vertebrate cell). The host cell, when cultured under appropriate conditions, synthesizes an UNC5H2d polypeptide that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

A number of suitable host cells are known in the art and many are available from the American Type Culture Collection (ATCC), Manassas, Va. Examples include, but are not limited to, mammalian cells, such as Chinese hamster ovary cells (CHO), CHO MFR(-) cells, human embryonic kidney (HEK) 293 or 293T cells, or 3T3 cells. The selection of suitable mammalian host cells and methods for transformation, culture, amplification, screening, product production, and purification are known in the art. Other suitable mammalian cell lines are the monkey COS-1 and COS-7 cell lines, and the CV-1 cell line. Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include but are not limited to, mouse neuroblastoma N2A cells, HeLa, mouse L-929 cells, 3T3 lines derived from Swiss, Balb-c or NIH mice, BHK, or HaK hamster cell lines. Each of these cell lines is known by and available to those skilled in the art of protein expression.

Similarly useful as host cells suitable for the present invention are bacterial cells. For example, the various strains of E. coli (e.g., HB101, DH5α, DH10, and MC1061) are well known as host cells in the field of biotechnology. Various strains of B. subtilis, Pseudomonas spp., other Bacillus spp., Streptomyces spp., and the like may also be employed in this method.

Many strains of yeast cells known to those skilled in the art are also available as host cells for the expression of the polypeptides of the present invention. Preferred yeast cells include, for example, Saccharomyces cerivisae and Pichia pastoris.

Additionally, where desired, insect cell systems may be utilized in the methods of the present invention. Such systems are described, for example, in Kitts et al., 1993, Biotechniques, 14:810-17. Preferred insect cells are Sf-9 and H3 (Invitrogen).

One may also use transgenic animals to express glycosylated UNC5H2d polypeptide. For example, one may use a transgenic milk-producing animal (a cow or goat, for example) and obtain the present glycosylated polypeptide in the animal milk. One may also use plants to produce the UNC5H2d polypeptide, however, in general, the glycosylation occurring in plants is different from that produced in mammalian cells, and may result in a glycosylated product which is not suitable for human therapeutic use. Polypeptide Production Host cells comprising an UNC5H2d polypeptide expression vector may be cultured using standard media well known to the skilled artisan. The media will usually contain all nutrients necessary for the growth and survival of the cells. Suitable media for culturing E. coli cells include, for example, Luria Broth (LB) and/or Terrific Broth (TB). Suitable media for culturing eukaryotic cells include Roswell Park Memorial Institute medium 1640 (RPMI 1640), Minimal Essential Medium (MEM) and/or Dulbecco's Modified Eagle Medium (DMEM), all of which may be supplemented with serum and/or growth factors as necessary for the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of transfected or transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline, and neomycin.

Purification and Isolation of the Polypeptide

The amount of an UNC5H2 polypeptide produced by a host cell can be evaluated using standard methods known in the art. Such methods include, without limitation Western blot analysis, SDS-polyacrylamide gel electrophoresis, non-denaturing gel is electrophoresis, High Performance Liquid Chromatography (HPLC) separation, immunoprecipitation, and/or activity assays such as DNA binding gel shift assays.

If an UNC5H2 polypeptide has been designed to be secreted from the host cells, the majority of polypeptide may be found in the cell culture medium. If however, the UNC5H2d polypeptide is not secreted from the host cells, it will be present in the cytoplasm and/or the nucleus (for eukaryotic host cells) or in the cytosol (for gram negative bacteria host cells).

For an UNC5H2 polypeptide situated in the host cell cytoplasm and/or nucleus (for eukaryotic host cells) or in the cytosol (for bacterial host cells), the intracellular material (including inclusion bodies for gram-negative bacteria) can be extracted from the host cell using any standard technique known to the skilled artisan. For example, the host cells can be lysed to release the contents of the periplasm/cytoplasm by French press, homogenization, and/or sonication followed by centrifugation.

If an UNC5H2 polypeptide has formed inclusion bodies in the cytosol, the inclusion bodies can often bind to the inner and/or outer cellular membranes and thus will be found primarily in the pellet material after centrifugation. The pellet material can then be treated at pH extremes or with a chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. The solubilized UNC5H2 polypeptide can then be analyzed using gel electrophoresis, immunoprecipitation, or the like. If it is desired to isolate the UNC5H2d polypeptide, isolation may be accomplished using standard methods such as those described herein and in Marston et al., 1990, Meth. Enz., 182:264-75.

In some cases, an UNC5H2 polypeptide may not be biologically active upon isolation. Various methods for “refolding” or converting the polypeptide to its tertiary structure and generating disulfide linkages can be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential is allowing for disulfide shuffling to occur in the formation of the protein's cysteine bridges. Some of the commonly used redox couples include cysteine/cystamine, glutathione (GSH)/dithiobis GSH, cupric chloride, dithiothreitol(DTT)/dithiane DTT, and 2-2-mercaptoethanol(bME)/dithio-b(ME). In many instances, a cosolvent may be used or may be needed to increase the efficiency of the refolding, and the more common reagents used for this purpose include glycerol, polyethylene glycol of various molecular weights, arginine and the like.

If inclusion bodies are not formed to a significant degree upon expression of an UNC5H2 polypeptide, then the polypeptide will be found primarily in the supernatant after centrifugation of the cell homogenate. The polypeptide may be further isolated from the supernatant using methods such as those described herein.

The purification of an UNC5H2d polypeptide from solution can be accomplished using a variety of techniques. If the polypeptide has been synthesized such that it contains a tag such as Hexa-histidine (UNC5H2d polypeptide/hexaHis) or other small peptide such as FLAG (Eastman Kodak Co., New Haven, Conn.) or myc (Invitrogen) at either its carboxyl- or amino-terminus, it may be purified in a one-step process by passing the solution through an affinity column where the column matrix has a high affinity for the tag. For example, poly-histidine binds with great affinity and specificity to nickel. Thus, an affinity column of nickel (such as the Qiagen nickel columns) can be used for purification of UNC5H2d polypeptide/polyHis.

Additionally, an UNC5H2d polypeptide may be purified through the use of a monoclonal antibody that is capable of specifically recognizing and binding to an UNC5H2d polypeptide.

Other suitable procedures for purification include, without limitation, affinity chromatography, immunoaffinity chromatography, ion exchange chromatography, molecular sieve chromatography, HPLC, electrophoresis (including native gel electrophoresis) followed by gel elution, and preparative isoelectric focusing (“Isoprime” machine/technique, Hoefer Scientific, San Francisco, Calif.). In some cases, two or more purification techniques may be combined to achieve increased purity.

An UNC5H2d polypeptide may also be prepared by chemical synthesis methods is (such as solid phase peptide synthesis) using techniques known in the art. Such polypeptides may be synthesized with or without a methionine on the amino-terminus. A chemically synthesized UNC5H2d polypeptide may be oxidized using methods set forth in these references to form disulfide bridges. A chemically synthesized UNC5H2d polypeptide is expected to have comparable biological activity to the corresponding UNC5H2d polypeptide produced recombinantly or purified from natural sources, and thus may be used interchangeably with a recombinant or natural UNC5H2d polypeptide.

Another means of obtaining an UNC5H2d polypeptide is via purification from biological samples such as source tissues and/or fluids in which the UNC5H2d polypeptide is naturally found. Such purification can be conducted using methods for protein purification as described herein. The presence of the UNC5H2d polypeptide during purification may be monitored, for example, using an antibody prepared against a recombinantly produced UNC5H2d polypeptide or peptide fragments thereof.

A number of additional methods for producing nucleic acids and polypeptides are known in the art, and the methods can be used to produce polypeptides having specificity for the UNC5H2d polypeptide. See, e.g., Roberts et al., 1997, Proc. Natl. Acad. Sci. U.S.A. 94:12297-303, which describes the production of fusion proteins between an mRNA and its encoded peptide. Additionally, U.S. Pat. No. 5,824,469 describes methods for obtaining oligonucleotides capable of carrying out a specific biological function. The procedure involves generating a heterogeneous pool of oligonucleotides, each having a 5′, randomized sequence, a central preselected sequence, and a 3′ randomized sequence. The resulting heterogeneous pool is introduced into a population of cells that do not exhibit the desired biological function. Subpopulations of the cells are then screened for those that exhibit a predetermined biological function. From that subpopulation, oligonucleotides capable of carrying out the desired biological function are isolated.

Processes for producing peptides or polypeptides are known in the art. This is done by producing stochastic genes or fragments thereof, and then introducing these genes into host cells, which produce one or more proteins, encoded by the stochastic genes. The host cells are then screened to identify those clones producing peptides or polypeptides having the desired activity.

Another method for producing peptides or polypeptides is described in PCT/IJS98/20094 (W099/15650) filed by Athersys, Inc. Known as “Random Activation of Gene Expression for Gene Discovery” (RAGE-GD), the process involves the activation of endogenous gene expression or over-expression of a gene by in situ recombination methods. For example, expression of an endogenous gene is activated or increased by integrating a regulatory sequence into the target cell that is capable of activating expression of the gene by non-homologous or illegitimate recombination. The target DNA is first subjected to radiation, and a genetic promoter inserted. The promoter eventually locates a break at the front of a gene, initiating transcription of the gene. This results in expression of the desired peptide or polypeptide.

It will be appreciated that these methods can also be used to create comprehensive UNC5H2d polypeptide expression libraries, which can subsequently be used for high throughput phenotypic screening in a variety of assays, such as biochemical assays, cellular assays, and whole organism assays (e.g., plant, mouse, etc.) synthesis. It will be appreciated by those skilled in the art that the nucleic acid and polypeptide molecules described herein may be produced by recombinant and other means.

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pro-, pro- or prepro-portion to produce an active mature polypeptide. In such polypeptides, the pre-, pro- or prepro-sequence may be a leader or secretory sequence or may be a sequence that is employed for purification of the mature polypeptide sequence.

Polypeptides may contain amino acids other than the 20 gene-encoded amino adds, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

Mutants of UNC5H2d, which contain amino acid substitutions, insertions or deletions, may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

In accordance with the present invention, any substitution should be preferably a “conservative” or “safe” amino acid substitution, which is commonly defined a substitution introducing an amino acids having sufficiently similar chemical properties (e.g. a basic, positively charged amino acid should be replaced by another basic, positively charged amino acid), in order to preserve the structure and the biological function of the molecule.

Preferably, one, two, three, four, five, ten, twenty, thirty or more conservative amino acid substitutions are made.

The literature provide many models on which the selection of conservative amino acids substitutions can be performed on the basis of statistical and physico-chemical studies on the sequence and/or the structure of proteins (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table I.

TABLE I

| | | More Preferred |
|---|---|---|
| Amino Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

Specific, non-conservative mutations can be also introduced in the polypeptides of the invention with different purposes. Mutations reducing the affinity of the UNC5H2d may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Preferred alternative, synonymous groups for amino acids derivatives included in peptide mimetics are those defined in Table II. A non-exhaustive list of amino acid derivatives also include aminoisobutyric acid (Aib), hydroxyproline (Hyp), 1,2,3,4-tetrahydro-isoquinoline-3-COOH, indoline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

TABLE II

| Amino Acid | Synonymous Groups |
|---|---|
| Ser | D-Ser, Thr, D-Thr, allo-Thr, Met, D-Met, Met(O), |
| | D-Met(O), L-Cys, D-Cys |
| Arg | D-Arg, Lys, D-Lys, homo-Arg, D-homo-Arg, Met, |
| | Ile, D-Met, D-Ile, Orn, D-Orn |
| Leu | D-Leu, Val, D-Val, AdaA, AdaG, Leu, D-Leu, Met, D-Met |
| Pro | D-Pro, L-l-thioazolidine-4-carboxylic acid, |
| | D-or L-1-oxazolidine-4-carboxylic acid |
| Thr | D-Thr, Ser, D-Ser, allo-Thr, Met, D-Met, Met(O), |
| | D-Met(O), Val, D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, |
| | Trans-3,4, or 5-phenylproline, AdaA, AdaG, cis-3,4, |
| | or 5-phenylproline, Bpa, D-Bpa |
| Tyr | D-Tyr, Phe, D-Phe, L-Dopa, His, D-His |
| Cys | D-Cys, S--Me--Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, |
| | D-Met, Ile, D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S-Me--Cys, Ile, D-Ile, Leu, D-Leu, Val, D-Val |

A derivative of a polypeptide comprises an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem AG, Switzerland; Bachem, USA).

Preferably, the derivative comprises at least one moiety attached to one or more functional groups, which occur as one or more side chains on the amino acid residues.

Preferably, the derivative is a water-soluble polymer.

Preferably, the water-soluble polymer is selected from the group consisting of polyethylene glycol, mono-methoxy polyethylene glycol, dextran, cellulose, poly-(N-vinyl pyrrolidone) polyethylene glycol, propylene glycol homopolymers, polypropylene oxide/ethylene oxide copolymers, polyoxyethylated polyols, and polyvinyl alcohol.

PEGylation may be carried out by known methods, such as the ones described in WO99/55377, for example.

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

The polypeptides of the invention also include fragments of the UNC5H2d polypeptides.

As used herein, the term “fragment” refers to a polypeptide having an amino acid sequence that is the same as part, but not all, of the amino acid sequence of the UNC5H2d polypeptide. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Fragments of the full length UNC5H2d polypeptides may consist of combinations of 1, 2, 3, 4 or more neighbouring exon sequences in the UNC5H2d polypeptide sequences, respectively. For example, such combinations include exons 1 and 2, exons 2 and 3 or exons 1 and 3, and so on. Such fragments are included in the present invention.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

Selective Binding Agents

The term “selective binding agent” refers to a molecule that has specificity for the UNC5H2d polypeptide. Suitable selective binding agents include, but are not limited to, antibodies and derivatives thereof, polypeptides, and small molecules. Suitable selective binding agents may be prepared using methods known in the art.

An exemplary UNC5H2d polypeptide selective binding agent of the present invention is capable of binding a certain portion of the UNC5H2d polypeptide thereby inhibiting the binding of the polypeptide to an UNC5H2 polypeptide receptor.

Selective binding agents such as antibodies and antibody fragments that bind the UNC5H2d polypeptide are within the scope of the present invention. The antibodies may be polyclonal including monospecific polyclonal; monoclonal (MAbs); recombinant; chimeric; humanized, such as complementarity-determining region (CDR)-grafted; human; single chain; and/or bispecific; as well as fragments; variants; or derivatives thereof. Antibody fragments include those portions of the antibody that bind to an epitope on the UNC5H2d polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies directed toward an UNC5H2d polypeptide generally are produced in animals (e.g., rabbits or mice) by means of multiple subcutaneous or intraperitoneal injections of the UNC5H2d polypeptide and an adjuvant. It may be useful to conjugate an UNC5H2d polypeptide to a carrier protein that is immunogenic in the species to be immunized, such as keyhole limpet hemocyanin, serum, albumin, bovine thyroglobulin, or soybean trypsin inhibitor. Also, aggregating agents such as alum are used to enhance the immune response. After immunization, the animals are bled and the serum is assayed for anti-UNC5H2d antibody titer.

Monoclonal antibodies directed toward an UNC5H2d polypeptide are produced using any method that provides for the production of antibody molecules by continuous cell lines in culture. Examples of suitable methods for preparing monoclonal antibodies include the hybridoma methods and the human B-ell hybridoma method. Also provided by the invention are hybridoma cell lines that produce monoclonal antibodies reactive with the UNC5H2d polypeptide.

Monoclonal antibodies of the invention may be modified for use as therapeutics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity.

In another embodiment, a monoclonal antibody of the invention is a “humanized” antibody. Generally, a humanized antibody has one or more amino acid residues introduced into it from a source that is non-human. Humanization can be performed, for example, using methods described in the art, by substituting at least a portion of a rodent complementarity-determining region for the corresponding regions of a human antibody.

The term “antibody” or “immunoglobulin” is intended to encompass both polyclonal and monoclonal antibodies. The preferred antibody is a monoclonal antibody reactive with the antigen. The term “antibody” is also intended to encompass mixtures of more than one antibody reactive with the antigen (e.g., a cocktail of different types of monoclonal antibodies reactive with the antigen). The term “antibody” is further intended to encompass whole antibodies, biologically functional fragments thereof, single-chain antibodies, and genetically altered antibodies such as chimeric antibodies comprising portions from more than one species, bifunctional antibodies, antibody conjugates, humanized and human antibodies. Biologically functional antibody fragments, which can also be used, are those peptide fragments derived from an antibody that are sufficient for binding to the antigen. Antibody as used herein is meant to include the entire antibody as well as any antibody fragments (e.g. F(ab′).sub.2, Fab′, Fab, Fv) capable of binding the epitope, antigen or antigenic fragment of interest.

By “purified antibody” is meant one which is sufficiently free of other proteins, carbohydrates, and lipids with which it is naturally associated. Such an antibody “preferentially binds” to UNC5h2d polypeptides of the present invention (or an antigenic fragment thereof, i.e., does not substantially recognize and bind to other antigenically unrelated molecules. A purified antibody of the invention is preferably immunoreactive with and immunospecific UNC5H2d of specific species and more preferably immunospecific for a native human UNC5H2d.

By “binds specifically” is meant high avidity and/or high affinity binding of an antibody to a specific polypeptide i.e., UNC5H2d. Antibody binding to its epitope on this specific polypeptide is preferably stronger than binding of the same antibody to any other epitope. Antibodies which bind specifically to UNC5H2d may be capable of binding other polypeptides at a weak, yet detectable, level (e.g., 10% or less of the binding shown to the polypeptide of interest). Such weak binding, or background binding, is readily discernible from the specific antibody binding to the compound or polypeptide of interest, e.g. by use of appropriate controls.

Preferably, the affinity is at least 1.5-fold, 2-fold, 5-fold 10-fold, 100-fold, 10³-fold, 10⁴-fold, 10⁵-fold or 10⁶-fold greater for a polypeptide of the invention than for other member known members of the UNC5 family.

The term “genetically altered antibodies” means antibodies wherein the amino acid sequence has been varied from that of a native antibody. Because of the relevance of recombinant DNA techniques to this invention, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

The term “humanized antibody” or “humanized immunoglobulin” refers to an immunoglobulin comprising a human framework, at least one and preferably all complimentarity determining regions (CDRs) from a non-human antibody, and in which any constant region present is substantially identical to a human immunoglobulin constant region, i.e., at least about 85-90%, preferably at least 95% identical. Hence, all parts of a humanized immunoglobulin, except possibly the CDRs, are substantially identical to corresponding parts of one or more native human immunoglobulin sequences. See, e.g. Queen et al., U.S. Pat. Nos. 5,5301,101; 5,585,089; 5,693,762; and 6,180,370 (each of which is incorporated by reference in its entirety).

“Fully humanized antibodies” are molecules containing both the variable and constant region of the human immunoglobulin. Fully humanized antibodies can be potentially used for therapeutic use, where repeated treatments are required for chronic and relapsing diseases such as autoimmune diseases. One method for the preparation of fully human antibodies consist of “humanization” of the mouse humoral immune system, i.e. production of mouse strains able to produce human Ig (Xenomice), by the introduction of human immunoglobulin (Ig) loci into mice in which the endogenous Ig genes have been inactivated. The Ig loci are exceedingly complex in terms of both their physical structure and the gene rearrangement and expression processes required to ultimately produce a broad immune response. Antibody diversity is primarily generated by combinatorial rearrangement between different V, D, and J genes present in the Ig loci. These loci also contain the interspersed regulatory elements, which control antibody expression, allelic exclusion, class switching and affinity maturation. Introduction of unrearranged human Ig transgenes into mice has demonstrated that the mouse recombination machinery is compatible with human genes. Furthermore, hybridomas secreting antigen specific hu-mAbs of various isotypes can be obtained by Xenomice immunisation with antigen.

Fully humanized antibodies and methods for their production are known in the art (Mendez et al., Nature Genetics 15:146-156 (1997); Buggemann et al., Eur. J. Immunol. 21:1323-1326 (1991); Tomizuka et al., Proc. Natl. Acad. Sci. USA 97:722-727 (2000) Patent WO 98/24893.

The term “chimeric antibody” refers to an antibody in which the constant region comes from an antibody of one species (typically human) and the variable region comes from an antibody of another species (typically rodent). Hence, chimeric antibodies are molecules of which different portions are derived from different animal species, such as those having the variable region derived from a murine Mab and a human immunoglobulin constant region. Chimeric antibodies are primarily used to reduce immunogenicity in application and to increase yields in production, for example, where murine Mabs have higher yields from hybridomas but higher immunogenicity in humans, such that human/murine chimeric Mabs are used. Chimeric antibodies and methods for their production are known in the art (Cabilly et al., Proc. Natl. Acad. Sci. USA 81:3273-3277 (1984); Morrison et al., Proc. Natl. Acad. Sci. USA 81:6851-6855 (1984); Boulianne et al., Nature 312:643-646 (1984); Cabilly et al., European Patent Application 125023 (published Nov. 14, 1984); Neuberger et al., Nature 314:268-270 (1985); Taniguchi et al., European Patent Application 171496 (published Feb. 19, 1985); Morrison et al., European Patent Application 173494 (published Mar. 5, 1986); Neuberger et al., PCT Application WO 8601533, (published Mar. 13, 1986); Kudo et al., European Patent Application 184187 (published Jun. 11, 1986); Sahagan et al., J. Immunol. 137:1066-1074 (1986); Robinson et al., International Patent Application No. WO8702671 (published May 7, 1987); Liu et al., Proc. Natl. Acad. Sci USA 84:3439-3443 (1987); Sun et al., Proc. Natl. Acad. Sci USA 84:214-218 (1987); Better et al., Science 240:1041-1043 (1988); Riechmann et al., Nature 332:323-327. and Harlow and Lane, ANTIBODIES: A LABORATORY MANUAL, supra. These references are entirely incorporated herein by reference.

As used herein, the phrase “antibody fragment” refers to a molecule comprising a portion of an antibody capable of specifically binding an antigen, an antigenic determinant or an epitope. It will be appreciated that Fab and F(ab′)2 and other fragments of the antibodies useful in the present invention may be used for the detection and quantitation of their antigens according to the methods disclosed herein for intact antibody molecules. Such fragments are typically produced by proteolytic cleavage, using enzymes such as papain (to produce Fab fragments) or pepsin (to produce F(ab′)2 fragments).

As regards the antibodies mentioned herein throughout, the term “monoclonal antibody” is meant to include monoclonal antibodies, chimeric antibodies, fully humanized antibodies, antibodies to anti-idiotypic antibodies (anti-anti-Id antibody) that can be labeled in soluble or bound form, as well as fragments thereof provided by any known technique, such as, but not limited to enzymatic cleavage, peptide synthesis or recombinant techniques. A monoclonal antibody contains a substantially homogeneous population of antibodies specific to antigens, which populations contain substantially similar epitope binding sites. Mabs may be obtained by methods known to those skilled in the art. See, for example Kohler and Milstein, Nature, 256:495-497 (1975); U.S. Pat. No. 4,376,110; Ausubel et al., eds., Harlow and Lane ANTIBODIES: A LABORATORY MANUAL, Cold Spring Harbor Laboratory (1988); and Colligan et al., eds., Current Protocols in Immunology, Greene Publishing Assoc. and Wiley Interscience N.Y., (1992-1996), the contents of which references are incorporated entirely herein by reference. Such antibodies may be of any immunoglobulin class including IgG, IgM, IgE, IgA, GILD and any subclass thereof. A hybridoma producing a mAb of the present invention may be cultivated in vitro, in situ or in vivo. Production of high titers of Mabs in vivo or in situ makes this the presently preferred method of production. The term “monoclonal antibody” is also meant to include both intact molecules as well as fragments thereof, such as, for example, Fab and F(ab′)2, which are capable of binding antigen. Fab and F(ab′)2 fragments lack the Fc fragment of intact antibody, clear more rapidly from the circulation, and may have less non-specific tissue binding than an intact antibody (Wahl et al., J. Nucl. Med. 24:316-325 (1983)).

A monoclonal antibody is said to be “capable of binding” a molecule if it is capable of specifically reacting with the molecule to thereby bind the molecule to the antibody.

An “antigen” is a molecule or a portion of a molecule capable of being bound by an antibody, which antigen is additionally capable of inducing an animal to produce antibody capable of binding to an epitope of that antigen. An antigen may have one or more than one epitope. The specific reaction referred to above is meant to indicate that the antigen will react, in a highly selective manner, with an epitope on its corresponding antibody and not with the multitude of other antibodies which may be evoked by other antigens.

The antibodies, including fragments of antibodies, useful in the present invention may be used to quantitatively or qualitatively detect their antigens in a sample or to detect presence of cells that express their antigens. This can be accomplished by immunofluorescence techniques employing a fluorescently labeled antibody (see below) coupled with fluorescence microscopy, flow cytometric, or fluorometric detection.

The antibodies (or fragments thereof) useful in the present invention may be employed histologically, as in immunofluorescence or immunoelectron microscopy, for in situ detection of their antigens. In situ detection may be accomplished by removing a histological specimen from a patient, and providing the labeled antibody of the present invention to such a specimen. The antibody (or fragment) is preferably provided by applying or by overlaying the labeled antibody (or fragment) to a biological sample. Through the use of such a procedure, it is possible to determine not only the presence of the antigens but also its distribution on the examined tissue. Using the present invention, those of ordinary skill will readily perceive that any of wide variety of histological methods (such as staining procedures) can be modified in order to achieve such in situ detection.

Such assays for the antigens typically comprises incubating a biological sample, such as a biological fluid, a tissue extract, freshly harvested cells such as lymphocytes or leukocytes, or cells which have been incubated in tissue culture, in the presence of a labeled antibody capable of identifying the antigens, and detecting the antibody by any of a number of techniques well known in the art.

The biological sample may be coupled to a solid phase support or carrier such as nitrocellulose, or other solid support or carrier which is capable of immobilizing cells, cell particles or soluble proteins. The support or carrier may then be washed with suitable buffers followed by treatment with a labeled antibody in accordance with the present invention, as noted above. The solid phase support or carrier may then be washed with the buffer a second time to remove unbound antibody. The amount of bound label on said solid support or carrier may then be detected by conventional means.

An antibody molecule of the present invention may be adapted for utilization in an immunometric assay, also known as a “two-site” or “sandwich” assay. In a typical immunometric assay, a quantity of unlabeled antibody (or fragment of antibody) is bound to a solid support or carrier and a quantity of detectably labeled soluble antibody is added to permit detection and/or quantitation of the temary complex formed between solid-phase antibody, antigen, and labeled antibody.

The antibodies of the invention can be used in connection with immunoaffinity chromatography technology. More specifically, the antibodies can be placed on the surface of a material within a chromatography column. Thereafter, a composition to be purified can be passed through the column. If the sample to be purified includes any UNC5H2d polypeptides which binds to the antibodies those UNC5H2d polypeptides will be removed from the sample and thereby purified.

Hence, in summary methods of diagnosis can be performed in vitro using a cellular sample (e.g., blood sample, lymph node biopsy or tissue) from a mammal or can be performed by in vivo imaging.

Compositions comprising the antibodies of the present invention can be used to detect the presence of UNC5H2d, for example, by radioimmunoassay, ELISA, FACS, etc. One or more labeling moieties can be attached to the humanized immunoglobulin. Exemplary labeling moieties include radiopaque dyes, radiocontrast agents, fluorescent molecules, spin-labeled molecules, enzymes, or other labeling moieties of diagnostic value, particularly in radiologic or magnetic resonance imaging techniques.

An IgG antibody preparation of the present invention may be advantageously purified from an anti-serum of the present invention using protein-G affinity purification, preferably via protein-G immunoprecipitation. An anti-serum derived from an animal immunized, can be used for detecting with optimal sensitivity, via Western immunoblotting analysis, Immunoprecipitation and ELISA, the UNC5H2d polypeptides.

In general, for applications benefiting from optimal reproducibility, standardization, or precision, a purified antibody or antibody fragment of the present invention capable of specifically binding the target antigen will generally be optimal relative to an unpurified preparation of the present invention.

It will be appreciated by the ordinarily skilled artisan that an antibody or antibody fragment having an affinity characterized by a dissociation constant of up to 10⁻¹² for a cognate antigen can be obtained using common art techniques.

As described hereinabove, the preparation may advantageously comprise an antibody or antibody fragment attached to any of various types of detectable molecule.

An antibody fragment has the advantage of being smaller than a parental antibody from which it is derived while retaining substantially identical target-antigen binding specificity, or both binding specificity and binding affinity, as the parental antibody. Thus, an antibody fragment, by virtue of being smaller than the parental antibody, will thereby generally have superior biodistribution, and diffusion properties (for example, systemically in-vivo, or in isolated tissues) than the latter. An antibody fragment substantially lacking an Fc region, such as a single-chain Fv, an Fab′, an Fab an F(ab′)₂ or a CDR, is advantageous for applications involving exposure of the preparation to a molecule capable of specifically binding such an Fc region, and in which such binding is undesirable. Typically this may involve an undesired binding of an Fc region exposed to a cognate Fc receptor, or an Fc-binding complement component (for example, complement component C1q, present in serum). Fc receptors are displayed on the surface of numerous immune cell types, including: professional APCs, such as dendritic cells; B lymphocytes; and granulocytes such as neutrophils, basophils, eosinophils, monocytes, macrophages, and mast cells. Thus, the absence of an Fc region from the antibody fragment may be particularly advantageous for avoiding undesired an Fc receptor-mediated immune cell activation or a complement component-mediated complement cascade, particularly when administering the preparation in-vivo to an individual.

An F(ab′)₂ is a fragment of an antibody molecule containing a divalent antigen-binding portion of an antibody molecule.

An F(ab′)₂ preparation of the present invention may be conveniently obtained using standard art methods by treating an antibody preparation of the present invention, such as an anti-serum of the present invention, with the enzyme pepsin. The resultant F(ab′)₂ product is a 5S particle.

An Fab, or Fab′ is a fragment of an antibody molecule containing a monovalent antigen-binding portion of an antibody.

The CDR can be generated e.g. as described in EP0585939 or as described by Strandberg et al. (Protein Eng. 2001 January; 14(1): 67-74). The CDR according to the invention can be a modified CDR, which has enhanced effect on the modulation of UNC5H2d polypeptide. An example for methods of modification of active peptides is described by Sawa et al. 1999 (J. Med. Chem. 42, 3289-3299).

An Fab′ preparation of the present invention may be conveniently obtained using standard art methods by treating an antibody preparation of the present invention, such as an anti-serum of the present invention, with the enzyme pepsin, followed by reduction of the resultant F(ab′)₂ into. Such reduction may be effected using a thiol reducing agent, and optionally using a blocking group for the sulfhydryl groups resulting from cleavage of disulfide linkages. Such treatment generates two monovalent 3.5S Fab's an Fc fragment. An Fab preparation may be conveniently obtained using standard art methods by treating an antibody preparation of the present invention, such as an anti-serum of the present invention, with the enzyme papain to yield the intact light chain and a portion of heavy chain composed of the variable and C_{H}1 domains.

Ample guidance for generating an antibody fragment by enzymatic treatment of an antibody is provided in the literature of the art (for example, refer to: Goldenberg, U.S. Pat. Nos. 4,036,945 and 4,331,647; Porter R R., 1959. Biochem J. 73:119-126).

A single chain Fv (also referred to in the art as “scFv”) is a single chain molecule including the variable region of the light chain and the variable region of the heavy chain, linked by a suitable polypeptide linker.

An F(ab′)₂, Fab′, Fab, or single-chain Fv or CDR preparation of the present invention may be obtained using recombinant techniques.

Obtaining a recombinant antibody fragment is effected by isolating mRNA of B lymphocytes of animals immunized with the target antigen, generating cDNA from the mRNA via RT-PCR, and using the cDNA to construct an antibody fragment phage-display library. B lymphocytes can be conveniently isolated from the spleen, or, alternately from the blood, bone-marrow, or lymph nodes of the immunized animal.

It will be appreciated that the above-described methodology can be used to obtain a monoclonal antibody fragment preparation of the present invention having essentially any desired target antigen-binding affinity and/or specificity. Such a preparation can be utilized in various applications benefiting from a reagent capable of binding the target antigen with such defined target antigen-binding characteristics.

Since an Fab′ is essentially similar in structure to an Fab, a preparation of the present invention comprising an Fab′ may be employed essentially interchangeably with one comprising an Fab, where such Fab′ and Fab comprise essentially the same heavy and light chain variable regions. For applications, as will usually be the case, benefiting from a preparation of the present invention comprising an antibody fragment capable of binding the target antigen with maximal affinity, an F(ab′)₂ preparation of the present invention may superior to an Fab, Fab′ or scFv preparation of the present invention, due to the divalent binding of an F(ab′)₂ to the target antigen relative to the monovalent binding of such a monovalent antibody fragment.

As mentioned hereinabove, depending on the application and purpose, the antibody or antibody fragment preparation may originate from any of various mammalian species

An antibody or antibody fragment preparation of the present invention originating from a desired species may be derived from serum of the animal of such species immunized with the target antigen.

A preparation of the present invention of a human or humanized antibody or antibody fragment may be preferable for applications involving administration of the preparation to an individual. For example, a human or humanized antibody or antibody fragment will generally tend to be optimally tolerated immunologically, and hence will display an optimal half-life in-vivo in a human, and will thereby display optimal effectiveness. Further guidance regarding production and exploitation of human or humanized antibodies is provided hereinbelow.

The preparation may be used per se or it can be formulated as an active ingredient in a pharmaceutical composition.

Thus, according to the present invention there is provided a pharmaceutical composition comprising a pharmaceutically acceptable carrier and, as an active ingredient, an antibody or antibody fragment of the present invention.

Methods of formulating the antibody or antibody fragment of the present invention as an active ingredient in a pharmaceutical composition, and methods of exploiting such a pharmaceutical composition are described hereinbelow.

Preferably, administering the antibody or antibody fragment is effected by administering the pharmaceutical composition of the present invention comprising the antibody or antibody fragment of the present invention as an active ingredient.

The antibody or antibody fragment is preferably administered so as to achieve a sufficient level of antibody fragment bound to the target antigen so as to achieve a desired regulation of the biochemical activity.

An ordinarily skilled artisan, such as a physician, more preferably a physician specialized in the disease, will possess the required expertise for determining a suitable therapeutic protocol, including a suitable route of administration, and a suitable dosage of the antibody or antibody fragment for effectively treating the disease according to the teachings of the present invention.

As described hereinabove, the target antigen (i.e. UNC5H2d), which is a polypeptide, may be obtained in various ways.

Preferably, the target antigen is obtained via standard chemical synthesis methodology.

The target antigen may be chemically synthesized using, for example, standard solid phase techniques. Such techniques include exclusive solid phase synthesis, partial solid phase synthesis methods, fragment condensation, classical solution synthesis. Solid phase polypeptide synthesis procedures are well known in the art [for example, refer to Stewart et al., in “Solid Phase Peptide Synthesis”, 2nd ed., Pierce Chemical Company, (1984)].

A synthetic polypeptide can be purified by preparative high performance liquid chromatography procedure, such as described by Creighton T. [Proteins, structures and molecular principles, W. H. Freeman and Co. N.Y. (1983)] and its amino acid sequence may be confirmed via standard amino acid sequencing procedures.

As described hereinabove, the preparation is preferably derived by immunizing a mammal with the target antigen.

Generating the preparation in-vivo may be advantageously effected by repeated injection of the target antigen into a mammal in the presence of adjuvant according to a schedule which boosts production of antibodies in the serum. In cases wherein the target antigen is too small to elicit an adequate immunogenic response (referred to as a “hapten” in the art), the hapten can be coupled to an antigenically neutral carrier such as keyhole limpet hemocyanin (KLH) or serum albumin [e.g., bovine serum albumin (BSA)] carriers (for example, refer to US. Pat. Nos. 5,189,178 and 5,239,078). Coupling a hapten to a carrier can be effected using various methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as cows, sheeps, mice, rabbits, and the like. Following in-vivo generation of an antibody, its serum titer in the host mammal can readily be measured using immunoassay procedures which are well known in the art.

As described hereinabove, the preparation may advantageously comprise a humanized antibody or antibody fragment.

Humanized antibodies or antibody fragments are genetically engineered chimeric antibodies or antibody fragments having-preferably minimal-portions derived from non human antibodies. Humanized antibodies include antibodies in which complementary determining regions of a human antibody (recipient antibody) are replaced by residues from a complementarity determining region of a non human species (donor antibody) such as mouse, rat or rabbit having the desired functionality. In some instances, Fv framework residues of the human antibody are replaced by corresponding non human residues. Humanized antibodies may also comprise residues which are found neither in the recipient antibody nor in the imported complementarity determining region or framework sequences. In general, the humanized antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanized antibodies optimally also include at least a portion of an antibody constant region, such as an Fc region, typically derived from a human antibody (see, for example, Jones et al., 1986. Nature 321:522-525; Riechmann et al., 1988. Nature 332:323-329; and Presta, 1992. Curr. Op. Struct. Biol. 2:593-596). Methods for humanizing non human antibodies or antibody fragments are well known in the art. Generally, a humanized antibody has one or more amino acid residues introduced into it from a source which is non human. These non human amino acid residues are often referred to as imported residues which are typically taken from an imported variable domain. Humanization can be essentially performed as described (see, for example: Jones et al., 1986. Nature 321:522-525; Riechmann et al., 1988. Nature 332:323-327; Verhoeyen et al., 1988. Science 239:1534-1536; U.S. Pat. No. 4,816,567) by substituting human complementarity determining regions with corresponding rodent complementarity determining regions. Accordingly, such humanized antibodies are chimeric antibodies, wherein substantially less than an intact human variable domain has been substituted by the corresponding sequence from a non human species. In practice, humanized antibodies may be typically human antibodies in which some complementarity determining region residues and possibly some framework residues are substituted by residues from analogous sites in rodent antibodies. Human antibodies or antibody fragments can also be produced using various techniques known in the art, including phage display libraries [see, for example, Hoogenboom and Winter, 1991. J. Mol. Biol. 227:381; Marks et al., 1991. J. Mol. Biol. 222:581; Cole et al., “Monoclonal Antibodies and Cancer Therapy”, Alan R. Liss, pp. 77 (1985); Boerner et al., 1991. J. Immunol. 147:86-95). Humanized antibodies can also be made by introducing sequences encoding human immunoglobulin loci into transgenic animals, e.g., into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to: U.S. Pat. Nos. 5,545,807, 5,545,806, 5,569,825, 5,625,126, 5,633,425, and 5,661,016; Marks et al., 1992. Bio/Technology 10:779-783; Lonberg et al., 1994. Nature 368:856-859; Morrison, 1994. Nature 368:812-13; Fishwild et al., 1996. Nature Biotechnology 14:845-51; Neuberger, 1996. Nature Biotechnology 14:826; Lonberg and Huszar, 1995. Intern. Rev. Immunol. 13:65-93).

Cell Source Identification Using an UNC5H2d Polypeptide

In accordance with certain embodiments of the invention, it may be useful to be able to determine the source of a certain cell type associated with an UNC5H2d polypeptide. For example, it may be useful to determine the origin of a disease or pathological condition as an aid in selecting an appropriate therapy. In certain embodiments, nucleic acids encoding an UNC5H2d polypeptide can be used as a probe to identify cells described herein by screening the nucleic acids of the cells with such a probe. In other embodiments, one may use anti-UNC5H2d polypeptide antibodies to test for the presence of an UNC5H2d polypeptide in cells, and thus, determine if such cells are of the types described herein.

UNC5H2d Polypeptide Compositions and Administration

Therapeutic compositions are within the scope of the present invention. Such UNC5H2d polypeptide pharmaceutical compositions may comprise a therapeutically effective amount of an UNC5H2d polypeptide or an UNC5H2d nucleic acid molecule in admixture with a pharmaceutically or physiologically acceptable formulation agent selected for suitability with the mode of administration. Pharmaceutical compositions may comprise a therapeutically effective amount of one or more UNC5H2d polypeptide selective binding agents in admixture with a pharmaceutically or physiologically acceptable formulation agent selected for suitability with the mode of administration.

The present invention also provides a pharmaceutical composition comprising (i) an UNC5H2d polypeptide, or a UNC5H2d nucleic acid molecule, or a vector according to the present invention, or a host cell according to the present invention, or a selective binding agent according to according to the present invention, or a composition according to according to the present invention, or a polypeptide comprising a derivative according to the present invention, or a fusion polypeptide according to the present invention (ii) and at least one pharmaceutically acceptable excipient. Excipients may be chosen from the below formulation materials.

Acceptable formulation materials preferably are nontoxic to recipients at the dosages and concentrations employed.

The pharmaceutical composition may contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropylbeta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counter ions (such as sodium), preservatives (such as benzalkonium chloride, benzoic acid, salicylic acid, phenethyl alcohol, methylparaben, propylparaben, chlorhexidine, sorbic acid, or hydrogen peroxide), 5 solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants.

The optimal pharmaceutical composition will be determined by a skilled artisan depending upon, for example, the intended route of administration, delivery format, and desired dosage. Such compositions may influence the physical state; stability, rate of in vivo release, and rate of in vivo clearance of the UNC5H2d molecule.

The primary vehicle or carrier in a pharmaceutical composition may be either aqueous or non-aqueous in nature. For example, a suitable vehicle or carrier for injection may be water, physiological saline solution, or artificial cerebrospinal fluid, possibly supplemented with other materials common in compositions for parenteral administration. Neutral buffered saline or saline mixed with serum albumin are further exemplary vehicles.

Other exemplary pharmaceutical compositions comprise Tris buffer of about pH 7.0-8.5, or acetate buffer of about pH 4.0-5.5, which may further include sorbitol or a suitable substitute. In one embodiment of the present invention, UNC5H2d polypeptide compositions may be prepared for storage by mixing the selected composition having the desired degree of purity with optional formulation agents in the form of a lyophilized cake or an aqueous solution. Further, the UNC5H2d polypeptide product may be formulated as a lyophilizate using appropriate excipients such as sucrose.

The UNC5H2d polypeptide pharmaceutical compositions can be selected for parenteral delivery. Alternatively, the compositions may be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in this invention may be in the form of a pyrogen-free, parenterally acceptable, aqueous solution comprising the desired UNC5H2d molecule in a pharmaceutically acceptable vehicle. A particularly suitable vehicle for parenteral injection is sterile distilled water in which an UNC5H2d molecule is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In one embodiment, a pharmaceutical composition may be formulated for inhalation. For example, UNC5H2d polypeptide may be formulated as a dry powder for inhalation. UNC5H2d polypeptide or nucleic acid molecule inhalation solutions may also be formulated with a propellant for aerosol delivery. In yet another embodiment, solutions may be nebulized. Pulmonary administration is further described in PCT Pub. No. WO 94120069, which describes the pulmonary delivery of chemically modified proteins.

It is also contemplated that certain formulations may be administered orally. In one embodiment of the present invention, the UNC5H2d polypeptide that is administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the UNC5H2d polypeptide. Diluents, flavorings, low melting point waxes, vegetable oils, lubricants, suspending agents, tablet disintegrating agents, and binders may also be employed.

Another pharmaceutical composition may involve an effective quantity of UNC5H2d polypeptides in a mixture with ion-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or tale.

Additional UNC5H2d polypeptide pharmaceutical compositions will be evident to those skilled ill the art, including formulations involving the UNC5H2d polypeptide in sustained- or controlled-delivery formulations. Techniques for formulating a variety of other sustained- or controlled-delivery means, such as liposome carriers, bio erodible microparticles or porous beads and depot injections, are also known to those skilled in the art.

Additional examples of sustained-release preparations include semipermeable polymer matrices in the form of shaped articles, e.g. films, or microcapsules.

Sustained release matrices may include polyesters, hydrogels, polylactides, copolymers of L-glutamic acid and gamma ethyl-L-glutamate, poly(2-hydroxyethyl-methacrylate), ethylene vinyl acetate or poly-D(-)-3-hydroxybutyric acid. Sustained-release compositions may also include liposomes, which can be prepared by any of several methods known in the art.

The UNC5H2d pharmaceutical composition to be used for in vivo administration typically must be sterile. This may be accomplished by filtration through sterile filtration membranes. Where the composition is lyophilized, sterilization using this method may be conducted either prior to, or following, lyophilization and reconstitution. The composition for parenteral administration may be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or as a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

The effective amount of an UNC5H2d pharmaceutical composition to be employed therapeutically will depend, for example, upon the therapeutic context and objectives.

One skilled in the art will appreciate that the appropriate dosage levels for treatment will thus vary depending, in part, upon the molecule delivered, the indication for which the UNC5H2d molecule is being used, the route of administration, and the size (body weight, body surface, or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. In other embodiments, the dosage may range from 0.1 mg/kg up to about 1 g/kg; or 5 mg/kg up to about 100 mg/kg.

The frequency of dosing will depend upon the pharmacokinetic parameters of the UNC5H2d molecule in the formulation being used. Typically, a clinician will administer the composition until a dosage is reached that achieves the desired effect.

The composition may therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions may be administered by bolus injection or continuously by infusion, or by implantation device.

Alternatively or additionally, the composition may be administered locally via implantation of a membrane, sponge, or other appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use UNC5H2d polypeptide pharmaceutical compositions in an ex vivo manner. In such instances, cells, tissues, or organs that have been removed from the patient are exposed to UNC5H2d polypeptide pharmaceutical compositions after which the cells, tissues, or organs are subsequently implanted back into the patient.

In other cases, an UNC5H2d polypeptide can be delivered by implanting certain cells that have been genetically engineered, using methods such as those described herein, to express and secrete the UNC5H2d polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods may be used to modify a cell that contains a normally transcriptionally-silent UNC5H2d gene, or an under-expressed gene, and thereby produce a cell, which expresses therapeutically efficacious amounts of the UNC5H2d polypeptide.

Homologous recombination is a technique originally developed for targeting genes to induce or correct mutations in transcriptionally active genes. The basic technique was developed as a method for introducing specific mutations into specific regions of the mammalian genome or to correct specific mutations within defective genes.

Through homologous recombination, the DNA sequence to be inserted into the genome can be directed to a specific region of the gene of interest by attaching it to targeting DNA. The targeting DNA is a nucleotide.

Therapeutic Uses

UNC5H2d nucleic acid molecules, polypeptides, and agonists and antagonists thereof can be used to treat, diagnose, ameliorate, or prevent a number of diseases, disorders, or conditions, including those recited herein.

The present patent application discloses an UNC5H2d polypeptide having several possible applications. In particular, whenever an increase in the UNC5H2 activity of a polypeptide of the invention is desirable in the therapy or in the prevention of a disease, reagents such as the disclosed UNC5H2d polypeptide, the corresponding fusion proteins and peptide mimetics, the encoding nucleic acids, the expressing cells, or the compounds enhancing their expression can be used.

Therefore, the present invention discloses pharmaceutical compositions for the treatment or prevention of diseases needing an increase in the UNC5H2 activity of a polypeptide of the invention, which contain one of the disclosed UNC5H2d polypeptides, the corresponding fusion proteins and peptide mimetics, the encoding nucleic acids, the expressing cells, or the compounds enhancing their expression, as active ingredient. The process for the preparation of these pharmaceutical compositions comprises combining the disclosed UNC5H2d polypeptides, the corresponding fusion proteins and peptide mimetics, the encoding nucleic acids, the expressing cells, or the compounds enhancing their expression, together with a pharmaceutically acceptable carrier. Methods for the treatment or prevention of diseases needing an increase in the UNC5H2 activity of a polypeptide of the invention, comprise the administration of a therapeutically effective amount of the disclosed UNC5H2d polypeptide, the corresponding fusion proteins and peptide mimetics, the encoding nucleic acids, the expressing cells, or the compounds enhancing their expression.

Amongst the reagents disclosed in the present patent application, the ligands, the antagonists or the compounds reducing the expression or the activity of polypeptides of the invention have several applications, and in particular they can be used in the therapy or in the diagnosis of a disease associated to the excessive UNC5H2 activity of a polypeptide of the invention.

Therefore, the present invention discloses pharmaceutical compositions for the treatment or prevention of diseases associated to the excessive UNC5H2 activity of a polypeptide of the invention, which contain one of the ligands, antagonists, or compounds reducing the expression or the activity of such polypeptides, as active ingredient. The process for the preparation of these pharmaceutical compositions comprises combining the ligand, the antagonist, or the compound, together with a pharmaceutically acceptable carrier. Methods for the treatment or prevention of diseases associated to the excessive UNC5H2 activity of the polypeptide of the invention, comprise the administration of a therapeutically effective amount of the antagonist, the ligand or of the compound. UNC5H2d nucleic acid molecule, polypeptide, and agonists and antagonists thereof can be used to treat, diagnose, ameliorate, or prevent a number of diseases, disorders, or conditions, including those recited herein.

UNC5H2d polypeptide agonists and antagonists include those molecules which regulate UNC5H2d polypeptide activity and either increase or decrease at least one activity of the mature form of the UNC5H2d polypeptide. The terms “inhibitor” or “antagonist” refer to molecules that alter partially or impair the functions and/or properties (such as receptor binding, lipid affinity, enzyme interaction, structural arrangement, synthesis, secretion, metabolism) of the natural protein. Agonists or antagonists may be co-factors, such as a protein, peptide, antibody, carbohydrate, lipid, or small molecular weight molecule, which interact with UNC5H2d polypeptide and thereby regulate its activity.

Potential polypeptide agonists or antagonists include antibodies that react with either soluble (UNC5H2d) or membrane-bound forms (UNC5H2a, UNC5H2b or UNC5H2c) of UNC5H2 polypeptides that comprise part or all of the extracellular domains of the said proteins. Molecules that regulate UNC5H2d polypeptide expression typically include nucleic acids encoding UNC5H2d polypeptide that can act as anti-sense regulators of expression.

Several domains have been identified in the UNC5H2d protein, which are indicators of UNC5H2d's associated diseases. Using SMART (Simple Modular Architecture Research Tool, which allows the identification and annotation of genetically mobile domains and the analysis of domain architectures, http://smart.embl-heidelberg.de/) and its OMIM (Online Mendelian Inheritance in Man, which is a database cataloging human genes and genetic disorders, http://www.ncbi.nlm.nih.gov/entrez/query.fcgi?db=OMIM) curated human diseases associated with missense mutations within domains, it can be retrieved human diseases associated with domains found in UNC5H2d (see example 3) and particularly those diseases that were found to be associated with two or more domains present in UNC5H2d or diseases that were associated with a secreted protein (as UNC5H2d is a secreted protein).

Lupus erythemasosus is found associated with the death domain, the immunoglobulin domain and the factor 1 membrane attack complex. As such UNC5H2d nucleic acid molecules, polypeptides, and agonists and antagonists thereof may be useful in diagnosing or treating Lupus erythemasosus.

Malaria is found associated with the immunoglobulin domain and the psalmodium circumsporozoite protein signature. As such UNC5H2d nucleic acid molecules, polypeptides, and agonists and antagonists thereof may be useful in diagnosing or treating malaria.

Lymphoproliferative syndrome, T-cell leukemia and multiple sclerosis are found associated with the death and immunoglobulin domains. As such UNC5H2d nucleic acid molecules, polypeptides, and agonists and antagonists thereof may be useful in diagnosing or treating lymphoproliferative syndrome, T-cell leukemia and multiple sclerosis.

Factor V deficiencies are found associated with the thrombospondin type 1 motif and the immunoglobulin domain. As such UNC5H2d nucleic acid molecules, polypeptides, and agonists and antagonists thereof may be useful in diagnosing or treating factor V deficiencies.

Complement component proteins deficiencies (C3, C6, C8 and C6/7 combined deficiencies) are found associated with the factor 1 membrane attack complex and the thrombospondin type 1 motif. As such UNC5H2d nucleic acid molecules, polypeptides, and agonists and antagonists thereof may be useful in diagnosing or treating complement component proteins deficiencies. Preferably, UNC5H2d nucleic acid molecules, polypeptides, and agonists and antagonists thereof may be useful in diagnosing or treating C3, C6, C8 and C6/7 combined deficiencies.

Asthma and ataxia telangiectasia are found associated with the immunoglobulin domain and the factor 1 membrane attack complex. As such UNC5H2d nucleic acid molecules, polypeptides, and agonists and antagonists thereof may be useful in diagnosing or treating asthma and ataxia telangiectasia.

Alzheimer's disease is found associated with the factor 1 membrane attack complex and the death domain. In addition, UNC5H2 is expressed in Alzheimer's disease brain and not in normal brain (example 4). As such UNC5H2d nucleic acid molecules, polypeptides, and agonists and antagonists thereof may be useful in diagnosing or treating Alzheimer's disease.

Leber optic atrophy is associated with the death domain and the rhodanese signature. As such UNC5H2d nucleic acid molecules, polypeptides, and agonists and antagonists thereof may be useful in diagnosing or treating Leber optic atrophy.

Hypercholesterolemia is found associated with the factor 1 membrane attack complex and the metallothionein domain. As such UNC5H2d nucleic acid molecules, polypeptides, and agonists and antagonists thereof may be useful in diagnosing or treating hypercholesterolemia.

Myopathy is found associated with the rhodanese signature and the factor 1 membrane attack complex. As such UNC5H2d nucleic acid molecules, polypeptides, and agonists and antagonists thereof may be useful in diagnosing or treating myopathy.

Properdin deficiencies are found associated with a secreted protein (properdin precursor or factor P). As such UNC5H2d nucleic acid molecules, polypeptides, and agonists and antagonists thereof may be useful in diagnosing or treating properdin deficiencies.

UNC5H expression is lost or reduced in many cancers including ovary tumors, breast tumors, uterus tumors, colorectal tumors, stomach tumors, lung tumors and kidney tumors (Thiébault et al.). As such UNC5H2d nucleic acid molecules, polypeptides, and agonists and antagonists thereof may be useful in diagnosing or treating cancer. Preferably, UNC5H2d nucleic acid molecules, polypeptides, and agonists and antagonists thereof may be useful in diagnosing or treating ovary tumors, breast tumors, uterus tumors, colorectal tumors, stomach tumors, lung tumors, kidney tumors, testicular tumors and esophageal cancer.

Since UNC5H2d polypeptide is likely to play a role in axonal guidance, UNC5H2d nucleic acid molecules, polypeptides, and agonists and antagonists thereof may be useful in diagnosing or treating diseases and conditions in which axonal guidance has a role. In addition, Lee S S et al. have identified two genes that are differentially expressed during development in human feta astrocytes (Lee S S et al. Yonsei Med J. Dec. 30, 2003;44(6):1059-68. “Characterization of the two genes differentially expressed during development in human fetal astrocytes.”). One gene, namely C8, has more than 96% homology to the 3′ end of integral UNC5H2 (48.7% overall). Astrocytes have known role in neurological disorders and autoimmune disease of the CNS such as Alzheimer's disease, Huntington's disease and also multiple sclerosis.

The expression of UNC5H2 in hippocampal aging and cognitive impairment was measured in the Gene Expression Omnibus (GEO) record GDS520 (http://www.ncbi.nlm.nih.gov/entrez/query.fcgi?db=geo). “GEO serves as a public repository for a wide range of high-throughput experimental data. These data include single and dual channel microarray-based experiments measuring mRNA, genomic DNA, and protein abundance, as well as non-array techniques such as serial analysis of gene expression (SAGE), and mass spectrometry proteomic data. In GDS85, identification of aging-dependent cognitive decline gene expression was performed from hippocampal CA1 tissue collected from 4, 14, and 24 months old male Fischer 344 rats after 7 days training on water maze and object memory task (single channel microarray experiment). Results clearly show a consistent increase in UNC5H2 expression in 14, and 24 months old rats compared with 4 months old rats. Thus, this is a further indication that UNC5H2 might be involved in cognitive impairment.

As such UNC5H2d nucleic acid molecules, polypeptides, and agonists and antagonists thereof may be useful in diagnosing or treating neurological disorders, autoimmune disorders of the CNS, Kallmann syndrome 1, cutis laxa-marfanoid syndrome, diabete mellitus, acanthosis nigricans, olfactory lobe agenesis, mirror hand movements (bimanual synkinesia), ataxia, bitemporal skull narrowing, hydrocephalus, cognitive impairment, dementia, crash syndrome, Masa syndrome, L1 or L1CAM disease, aniridia type II, leprechaunism, Rabson-Mendenhall syndrome, schizophrenia, Alzheimer's disease, cerebral ischemia, dyslexia, Parkinson's disease, Wiskott-Aldrich syndrome, Bardet-Biedl syndrome, Angelman syndrome, multiple sclerosis, Huntington's disease and Prader-Willi syndrome. UNC5H2d nucleic acid molecules, polypeptides, and agonists and antagonists may also be useful in axon regrowth following injury or diseases, in cortical development, in retinal ganglion cells regeneration and development. Other diseases and conditions associated with axonal guidance are encompassed within the scope of this invention.

Since UNC5H2 polypeptide is a direct transcriptional target for the tumor suppressor p53 and mediates p53 proapoptotic activity, UNC5H2d nucleic acid molecules, polypeptides, and agonists and antagonists thereof may be useful in diagnosing or treating diseases and conditions in which p53 has been associated. As such UNC5H2d nucleic acid molecules, polypeptides, and agonists and antagonists thereof may be useful in diagnosing or treating Li-Fraumeni syndrome, neurofibromatosis, breast cancer (see example 4), acute promyelocytic leukemia, hepatocellular carcinoma, adenomatous leukemia, hepatocellular carcinoma, glioma of the brain, nasopharyngeal carcinoma, glioma of the brain, pancreatic carcinoma, Fanconi anemia, colorectal cancer, Bloom syndrome, adrenocortical carcinoma, Huntington disease, ataxia-telangiectasia, medulloblastoma, retinoblastoma, xeroderma pigmentosum, Wilson disease, Werner disease, hemochromatosis, excision-repair cross-complementary rodent repair deficiency, bladder cancer, papilloma of choroid plexus, testicular tumors, Miller-Dieker lissencephaly syndrome, prostate cancer, Peutz-Jeghers syndrome, ectodermal dysplasia, CYP1A1 deficiency, Angelman syndrome, rhabdoid tumor, SMARCB1 deficiency, pleuropulmonary blastoma, Fanconi anemia, lung cancer (see example 4), Wilms tumor 1, prohibitin deficiency, phospholipase A2 deficiency, Cowden disease, mesothelioma, B-cell lymphoma, colon cancer, neurofibromatosis, Turcot syndrome, thyroid hormone receptor deficiency, gastric cancer, liver cancer (see example 4), esophageal cancer and ankyloblepharon-ectodermal defects. Other diseases and conditions associated with p53 are encompassed within the scope of this invention. The proapoptotic activity of UNC5H2d might depend on the caspase cleavage of UNC5h2d and the conserved death domain located at the C terminus.

Since UNC5H2d polypeptide expression has been detected in rheumatoid arthritis synovium, osteoarthritic synovium, human lupus kidney, human lupus spleen, human lupus liver, as well as in Crohn's small intestine and ulcerative colitis intestine but not in normal small intestine (example 4), UNC5H2d nucleic acid molecules, polypeptides, and agonists and antagonists thereof may be useful in diagnosing or treating rheumatoid arthritis, lupus (SLE), osteoarthritis, Crohn's disease and ulcerative colitis and more generally in treating, preventing and/or diagnosing inflammation.

Since UNC5H2 polypeptide expression has been detected in skeletal muscle, UNC5H2d nucleic add molecules, polypeptides, and agonists and antagonists thereof may be useful in diagnosing or treating diseases and conditions affecting skeletal muscle. Examples of such diseases and conditions include, but are not limited to, cachexia and muscular dystrophy. Other diseases and conditions associated with skeletal muscle development and function are encompassed within the scope of this invention.

Since UNC5H2d polypeptide expression has been detected in the uterus, UNC5H2d nucleic add molecules, polypeptides, and agonists and antagonists thereof may be useful in diagnosing or treating diseases and conditions affecting the uterus. Examples of such diseases and conditions include, but are not limited to, miscarriage, endometriosis, uterine cancer, and female infertility. Other diseases and conditions associated with uterus development and function are encompassed within the scope of this invention.

Since UNC5H2d polypeptide expression has been detected in the bone marrow, UNC5H2d nucleic acid molecules, polypeptides, and agonists and antagonists thereof may be useful in diagnosing or treating diseases and conditions affecting the bone marrow. Examples of such diseases and conditions include, but are not limited to, leukemia. Other diseases and conditions associated with the development and function of the bone marrow are encompassed within the scope of this invention.

Since UNC5H2d polypeptide expression has been detected in the kidney (e.g. fetal kidney) and human lupus kidney, UNC5H2d nucleic acid molecules, polypeptides, and agonists and antagonists thereof may be useful in diagnosing or treating diseases and conditions affecting the kidney. Examples of such diseases and conditions include, but are not limited to, Lupus (SLE), polycystic kidney disease, glomerulocystic or medullary kidney disease, serpentine fibula-polycystic kidney syndrome, kidney dysplasia. Other diseases and conditions associated with the development and function of the kidney are encompassed within the scope of this invention.

Since UNC5H2d polypeptide expression has been detected in the ovaries, UNC5H2d nucleic acid molecules, polypeptides, and agonists and antagonists thereof may be useful in diagnosing or treating diseases and conditions affecting the ovaries. Examples of such diseases and conditions include, but are not limited to, female infertility and ovarian cancer. Other diseases and conditions associated with the development and function of the ovaries are encompassed within the scope of this invention.

Since UNC5H2 polypeptide expression has been detected in the thyroid, UNC5H2d nucleic acid molecules, polypeptides, and agonists and antagonists thereof may be useful in diagnosing or treating diseases and conditions affecting the thyroid. Examples of such diseases and conditions include, but are not limited to, thyroid dysgenesis, thyroid carcinoma, candidiasis, Hashimoto thyroiditis, hyperthyroidism, thyroid adenoma. Other diseases and conditions associated with the development and function of the thyroid are encompassed within the scope of this invention.

Since UNC5H2d polypeptide expression has been detected in the stomach and small intestine, UNC5H2d nucleic acid molecules, polypeptides, and agonists and antagonists thereof may be useful in diagnosing or treating diseases and conditions affecting the stomach. Examples of such diseases and conditions include, but are not limited to, polyposis, Peutz-Jeghers syndrome, adenomatous polyposis, visceral myopathy, intestinal atresis, pylovic atresia, volvulus, gastric cancer, basal cell aerus syndrome and Osler-Rendu-Weber syndrome 2. Other diseases and conditions associated with the development and function of the stomach and small intestine are encompassed within the scope of this invention.

Since UNC5H2d polypeptide expression has been detected in the colon, UNC5H2d nucleic acid molecules, polypeptides, and agonists and antagonists thereof may be useful in diagnosing or treating diseases and conditions affecting the colon. Examples of such diseases and conditions include, but are not limited to, adenomatus polyposis, colorectal cancer, colon cancer, Turcot syndrome, leukemia, Lynch cancer and colonic atresia. Other diseases and conditions associated with the development and function of the colon are encompassed within the scope of this invention.

Since UNC5H2d polypeptide expression has been detected in the lung (e.g. lung focal fibrosis), UNC5H2d nucleic acid molecules, polypeptides, and agonists and antagonists thereof may be useful in diagnosing or treating diseases and conditions affecting the lung. Examples of such diseases and conditions include, but are not limited to lung focal fibrosis, lung cancer, pneumonitis, lung agenesis, cystic disease of the lung, cystic fibrosis and Li-Fraumeni syndrome. Other diseases and conditions associated with the development and function of the lung are encompassed within the scope of this invention.

Since UNC5H2d polypeptide expression has been detected in the cirrhosis spleen and normal spleen, UNC5H2d nucleic acid molecules, polypeptides, and agonists and antagonists thereof may be useful in diagnosing or treating diseases and conditions affecting the spleen. Examples of such diseases and conditions include, but are not limited to, cirrhosis spleen, reticulosis, asplenia, Roberts syndrome, Meckel syndrome, cephalin lipidosis and Gaucher disease. Other diseases and conditions associated with the development and function of the spleen are encompassed within the scope of this invention.

Since UNC5H2 polypeptide expression has been detected in the bladder, UNC5H2d nucleic acid molecules, polypeptides, and agonists and antagonists thereof may be useful in diagnosing or treating diseases and conditions affecting the bladder. Examples of such diseases and conditions include, but are not limited to, bladder cancer, exstrophy of bladder, bladder diverticulum, Wolfram syndrome and Costello syndrome. Other diseases and conditions associated with the development and function of the bladder are encompassed within the scope of this invention.

Since UNC5H2d polypeptide expression has been detected in the mammary gland and breast, UNC5H2d nucleic acid molecules, polypeptides, and agonists and antagonists thereof may be useful in diagnosing or treating diseases and conditions affecting the breast. Examples of such diseases and conditions include, but are not limited to, breast cancer, ataxia-telangiectasia, Cowden disease and Li-Fraumeni syndrome. Other diseases and conditions associated with the development and function of the breast are encompassed within the scope of this invention.

Since UNC5H2d polypeptide expression has been detected in the heart (e.g. fetal heart), UNC5H2d nucleic acid molecules, polypeptides, and agonists and antagonists thereof may be useful in diagnosing or treating diseases and conditions affecting the heart. Examples of such diseases and conditions include, but are not limited to, heart block, Holt-Oram syndrome, choanal atresia and cardiac valvular dysplasia. Other diseases and conditions associated with the development and function of the heart are encompassed within the scope of this invention.

Since UNC5H2d polypeptide expression has been detected in the cartilage (e.g. osteoarthritic cartilage knee), UNC5H2d nucleic add molecules, polypeptides, and agonists and antagonists thereof may be useful in diagnosing or treating diseases and conditions affecting the cartilage. Examples of such diseases and conditions include, but are not limited to, metaphyseal dysplasia, cartilage-hair hypoplasia, pseudoachondroplastic dysplasia, Keutel syndrome, diastrophic or epiphyseal dysplasia, synovial chondromatosis and achondrogenosis. Other diseases and conditions associated with the development and function of the cartilage are encompassed within the scope of this invention.

Since UNC5H2 polypeptide expression has been detected in the hematopoietic tissues, UNC5H2d nucleic acid molecules, polypeptides, and agonists and antagonists thereof may be useful in diagnosing or treating diseases and conditions affecting the hematopoietic tissues. Examples of such diseases and conditions include, but are not limited to, Huntington disease, neurofibromatosis and Krabbe disease. Other diseases and conditions associated with the development and function of the hematopoietic tissues are encompassed within the scope of this invention.

Since UNC5H2 polypeptide expression has been detected in the immune tissues, UNC5H2d nucleic acid molecules, polypeptides, and agonists and antagonists thereof may be useful in diagnosing or treating diseases and conditions affecting the immune tissues. Examples of such diseases and conditions include, but are not limited to, ataxia-telangiectasia, Digeorge syndrome and Huntington disease. Other diseases and conditions associated with the development and function of the immune tissues are encompassed within the scope of this invention.

Since UNC5H2d polypeptide expression has been detected in the brain (e.g. neurons, neuroblastoma, adrenal cortex carcinoma), UNC5H2d nucleic acid molecules, polypeptides, and agonists and antagonists thereof may be useful in diagnosing or treating diseases and conditions affecting the neurons and brain. Examples of such diseases and conditions include, but are not limited to neuroblastoma, adrenal cortex carcinoma, Pick disease, glioma, neuropathies, dementias and multiple sclerosis. Other diseases and conditions associated with the development and function of the brain are encompassed within the scope of this invention.

Since UNC5H2d polypeptide expression has been detected in the synovial membrane, UNC5H2d nucleic acid molecules, polypeptides, and agonists and antagonists thereof may be useful in diagnosing or treating diseases and conditions affecting the synovial membrane. Examples of such diseases and conditions include, but are not limited to synovial chondromatosis and Bartter syndrome Other diseases and conditions associated with the development and function of the synovial membrane are encompassed within the scope of this invention.

Since UNC5H2 polypeptide expression has been detected in the amygdala, UNC5H2d nucleic acid molecules, polypeptides, and agonists and antagonists thereof may be useful in diagnosing or treating diseases and conditions affecting the amygdala. Examples of such diseases and conditions include, but are not limited to, frontotemporal dementia and lipoid proteinosis of Urbach and Wiethe. Other diseases and conditions associated with the development and function of the amygdala are encompassed within the scope of this invention.

Since UNC5H2 polypeptide expression has been detected in the prostate, UNC5H2d nucleic acid molecules, polypeptides, and agonists and antagonists thereof may be useful in diagnosing or treating diseases and conditions affecting the prostate. Examples of such diseases and conditions include, but are not limited to, prostate cancer, androgen insensitivity syndrome Li-Fraumeni syndrome and Cowden disease. Other diseases and conditions associated with the development and function of the prostate are encompassed within the scope of this invention.

Since UNC5H2d polypeptide expression has been detected in the pancreas (e.g. purified pancreatic islet), UNC5H2d nucleic acid molecules, polypeptides, and agonists and antagonists thereof may be useful in diagnosing or treating diseases and conditions affecting the pancreas. Examples of such diseases and conditions include, but are not limited to, nesidioblastosis, Pearson Marrow syndrome, Shwachman-Diamond syndrome and asplenia. Other diseases and conditions associated with the development and function of the pancreas are encompassed within the scope of this invention.

Since UNC5H2 polypeptide expression has been detected in the bone (e.g. subchondral bone, chondrosarcoma), UNC5H2d nucleic acid molecules, polypeptides, and agonists and antagonists thereof may be useful in diagnosing or treating diseases and conditions affecting the bone. Examples of such diseases and conditions include, but are not limited to chondrosarcoma, bone cysts, chondrodysplasia, Paget disease and Bruck syndrome. Other diseases and conditions associated with the development and function of the bone are encompassed within the scope of this invention.

Since UNC5H2d polypeptide expression has been detected in the placenta, UNC5H2d nucleic acid molecules, polypeptides, and agonists and antagonists thereof may be useful in diagnosing or treating diseases and conditions affecting the placenta. Examples of such diseases and conditions include, but are not limited to, circumvallate placenta syndrome, ichthyosis and Neu-laxova syndrome. Other diseases and conditions associated with the development and function of the placenta are encompassed within the scope of this invention.

Since UNC5H2 polypeptide expression has been detected in melanocytes (e.g. pooled human melanocytes), UNC5H2d nucleic acid molecules, polypeptides, and agonists and antagonists thereof may be useful in diagnosing or treating diseases and conditions related to melanocytes. Examples of such diseases and conditions include, but are not limited to Wardenburg syndrome, B cell lymphoma, hair color 2, osteopetrosis, Elejalde syndrome, nephropathic systinosis, Griscelli syndrome, oculocutaneous albinism, Peutz-Jeghers syndrome, Piebald trait, Hermansky-Pudlak syndrome, neurofibromatosis. Other diseases and conditions associated with the development and function of melanocytes are encompassed within the scope of this invention.

Since UNC5H2 polypeptide expression has been detected in large cell carcinoma, UNC5H2d nucleic acid molecules, polypeptides, and agonists and antagonists thereof may be useful in diagnosing or treating diseases and conditions related to large cell carcinomas. Examples of such diseases and conditions include, but are not limited to, basal cell nevus syndrome, thyroid carcinoma, hepatocellular carcinoma and multiple endocrine neoplasia. Other diseases and conditions associated with large cell carcinomas are encompassed within the scope of this invention.

Since UNC5H2 polypeptide expression has been detected in the neck, UNC5H2d nucleic acid molecules, polypeptides, and agonists and antagonists thereof may be useful in diagnosing or treating diseases and conditions affecting the neck. Examples of such diseases and conditions include, but are not limited to, Kousseff syndrome, Nevus flammeus of Nape, Pterygium syndrome and Costello syndrome. Other diseases and conditions associated with the development and function of the neck are encompassed within the scope of this invention.

Since UNC5H2d polypeptide expression has been detected in the liver, the cervix, the skin, the salivary gland, the adrenal gland and the eye, UNC5H2d nucleic acid molecules, polypeptides, and agonists and antagonists thereof may be useful in diagnosing or treating diseases and conditions affecting the liver, the cervix, the skin, the salivary gland, the adrenal gland and the eye.

Since UNC5H2d polypeptide expression has been detected in atherosclerotic plaques, UNC5H2d nucleic acid molecules, polypeptides, and agonists and antagonists thereof may be useful in diagnosing or treating diseases atherosclerosis.

Agonists or antagonists of the UNC5H2d polypeptide function may be used (simultaneously or sequentially) in combination with one or more cytokines. growth factors, antibiotics, anti-inflammatories, and/or chemotherapeutic agents as is appropriate for the condition being treated.

In a preferred embodiment, the UNC5H2d polypeptide is used for the treatment and/or prevention of cardiovascular diseases and/or hematology-related disorders.

Preferably, the cardiovascular disorder is selected from cardiac and respiratory arrest, valvular heart disease, arterial hypertension, endocarditis, orthostatic hypotension, syncope, pericardial disease, arteriosclerosis, cardiac tumor, coronary artery disease, disease of the aorta and its branches, heart failure, peripheral vascular disorder, shock, athletic heart syndrome or arrhythmia.

Preferably, the hematology-related disorder is selected from anemia, histiocytic syndrome, iron overload related disorder, leukemia, lymphoma, myeloproliferative disorder, plasma cell dyscrasia, hemostasis and coagulation disorder, disorder of the spleen, thrombotic disorder, platelet disorder, vascular bleeding disorder, leukopenia, lymphocytopenia or AIDS-associated hematologic disorder and malignancy.

Other diseases or disorders caused by or mediated by undesirable levels of the UNC5H2d polypeptide are encompassed within the scope of the invention. Undesirable levels include excessive levels of the UNC5H2d polypeptide and sub-normal levels of the UNC5H2d polypeptide.

Uses of UNC5H2d Nucleic Acid and Polypeptide

Nucleic acid molecules of the invention (including those that do not themselves encode biologically active polypeptides) may be used to map the locations of the UNC5H2d gene and related genes on chromosomes. Mapping may be done by techniques known in the art, such as PCR amplification and in situ hybridization.

UNC5H2d nucleic acid molecules (including those that do not themselves encode biologically active polypeptides), may be useful as hybridization probes in diagnostic assays to test, either qualitatively or quantitatively, for the presence of an UNC5H2d nucleic acid molecule in mammalian tissue or bodily fluid samples.

Other methods may also be employed where it is desirable to inhibit the activity of one or more UNC5H2d polypeptides. Such inhibition may be effected by nucleic acid molecules that are complementary to and hybridize to expression control sequences (triple helix formation) or to UNC5H2d mRNA. For example, antisense DNA or RNA molecules, which have a sequence that is complementary to at least a portion of an UNC5H2d gene can be introduced into the cell. Anti-sense probes may be designed is by available techniques using the sequence of the UNC5H2d gene disclosed herein. Typically, each such antisense molecule will be complementary to the start site (5′ end) of each selected UNC5H2d gene. When the antisense molecule then hybridizes to the corresponding UNC5H2d mRNA, translation of this mRNA is prevented or reduced. Anti-sense inhibitors provide information relating to the decrease or absence of an UNC5H2d polypeptide in a cell or organism.

Alternatively, gene therapy may be employed to create a dominant-negative inhibitor of the UNC5H2d polypeptide. In this situation, the DNA encoding a mutant polypeptide of the UNC5H2d polypeptide can be prepared and introduced into the cells of a patient using either viral or non-viral methods as described herein. Such mutant is typically designed to compete with endogenous polypeptide in its biological role.

In addition, an UNC5H2d polypeptide, whether biologically active or not, may be used as an immunogen, that is, the polypeptide contains at least one epitope to which antibodies may be raised. Selective binding agents that bind to an UNC5H2d polypeptide (as described herein) may be used for in vivo and in vitro diagnostic purposes, including, but not limited to, use in labeled form to detect the presence of an UNC5H2d polypeptide in a body fluid or cell sample. The antibodies may also be used to prevent, treat, or diagnose a number of diseases and disorders, including those recited herein. The antibodies may bind to an UNC5H2d polypeptide so as to diminish or block at least one activity characteristic of the UNC5H2d polypeptide, or may bind to a polypeptide to increase at least one activity characteristic of an UNC5H2d polypeptide (including by increasing the pharmacokinetics of the UNC5H2d polypeptide).

The UNC5H2d polypeptide can be used to clone UNC5H2d ligands using an “expression cloning” strategy Radiolabeled (125 Iodine). The UNC5H2d polypeptide or an “affinity/activity tagged” UNC5H2d polypeptide (such as an Fc fusion or an alkaline phosphatase fusion) can be used in binding assays to identify a cell type, cell line, or tissue that expresses an UNC5H2d ligand. RNA isolated from such cells or tissues can then be converted to cDNA, cloned into a mammalian expression vector, and transfected into mammalian cells (e.g., COS or 293) to create an expression library. A radiolabeled or tagged UNC5H2d polypeptide can then be used as an affinity reagent to identify and isolate the subset of cells in this library expressing an UNC5H2d ligand. DNA is then isolated from these cells and transfected into mammalian cells to create a secondary expression library in which the fraction of cells expressing the UNC5H2d ligand would be many-fold higher is than in the original library. This enrichment process can be repeated iteratively until a single recombinant clone containing the UNC5H2d ligand is isolated. Isolation of UNC5H2d ligands is useful for identifying or developing novel agonists and antagonists of the UNC5H2d signaling pathway. Such agonists and antagonists include UNC5H2d ligands, antiUNC5H2d ligand antibodies, small molecules or antisense oligonucleotides.

The human UNC5H2d nucleic acids of the present invention, as well as murine UNC5H2d nucleic acids, are also useful tools for isolating the corresponding chromosomal UNC5H2d polypeptide genes. For example, mouse chromosomal DNA containing the UNC5H2d sequence can be used to construct knockout mice, thereby permitting an examination of the in vivo role for the UNC5H2d polypeptide. The human UNC5H2d genomic DNA can be used to identify heritable tissue-degenerating diseases.

Preferably, the UNC5H2d polypeptides of the present invention are used for the preparation of a medicament for the treatment and/or prevention of disease.

Similarly to other members of the UNC5H family, UNC5H2d may display three major functions by means of its modulation of netrin-1 binding to UNC5H2 receptors and/or direct interaction with membrane bound forms:
1) For example, it may modulate the migration of neurons and their axons towards or from midline cells either favouring attraction or repulsion of axons. For example, in the presence of UNC5H2d, one category of axons may be repelled from the midline, whereas a subset of axons may be attracted towards the midline. Thus, UNC5H2d activity can for example be confirmed by measuring axons attraction or repulsion from or towards the midline cells. Without wishing to be bound to theory, UNC5H2d, to favour attraction, is able to prevent or disrupt the netrin-1 dependent UNC5H-DCC complex formed to mediate repulsion. A region of UNC5H2d which may be implicated in this modulating activity corresponds to the extracellular part of membrane bound UNC5H2, i.e. spans from amino acids 1 to 355 of UNC5H2d. As such, the present invention also encompasses the use of UNC5H2d or a fragment thereof, e.g. spanning from amino acids 1 to 355 or from amino acids 26 to 355 of UNC5H2d in the modulation of axons migration. Diseases implicated in the migration of axons have already been disclosed supra.
Preferably, the invention relates to a soluble UNC5H2 and its use in the modulation of axons migration.
Preferably, the invention relates to an UNC5H2d polypeptide and its use in the modulation of axons migration.
Preferably, the invention relates a fragment of UNC5H2d spanning from amino acids 1 to 355 and its use in the modulation of axons migration.
Preferably, the invention relates to a fragment of UNC5H2d spanning from amino acids 26 to 355 and its use in the modulation of axons migration.
Preferably, the invention relates to a fragment of UNC5H2d consisting of the two immunoglobulin domains and the two thrombospondin domains of UNC5H2d and its use in the modulation of axons migration.
Preferably, the invention relates to a fragment of UNC5H2d consisting of the two immunoglobulin domains of UNC5H2d and its use in the modulation of axons migration.
2) Furthermore, UNC5H2d may modulate cell death and act as a tumour suppressor. UNC5H2d may prevent or disrupt netrin-1 binding to membrane bound UNC5H2. UNC5H2d might therefore antagonize netrin-1's activity and have pro-apoptotic activity. Thus, UNC5H2d activity can for example be confirmed by measuring cell death or pro-apoptotic activity by either treatment with a general and potent caspase inhibitor (e.g. zVAD-fmk) in the inhibition of anchorage-independent growth and invasive ability or either by colorectal LS174T, Ras-tranformed NIH 3T3, or large T-transformed 293 cells transiently transfected with UNC5H2d-expressing constructs with or without addition of netrin-1 and allowed to grow in soft agar or invade through a reconstituted 3D basement membrane gel (Matrigel). The colorectal LS174T or glioblastoma U373MG cells can also be transfected with p53 and analyzed for growth in soft agar and invasion in Matrigel. Activity can also be confirmed by inhibition of UNC5H2d expression by AS3 to block apoptosis induced by DNA damage after exposure to adriamycin. UNCH5H2d activity can also be confirmed by addition of UNC5H2d or a fragment thereof along with a GST-Netrin-1 inhibition of Ad-p53-induced apoptosis. A region of UNC5H2d which may be implicated in this stimulating activity corresponds to the extracellular part of membrane bound UNC5H2, i.e. spans from amino acids 1 to 355 of UNC5H2d. As such, the present invention also encompasses the use of a fragment of UNC5H2d, i.e. spanning from amino acids 1 to 355 or from amino acids 26 to 355 of UNC5H2d in stimulating apoptosis. Thus, the present invention also relates to the use of UNC5H2d or a fragment thereof in the treatment or prevention of cancer. Different types of cancers have already been disclosed supra.
Preferably, the invention relates to a soluble UNC5H2 and its use in stimulating apoptosis.
Preferably, the invention relates to an UNC5H2d polypeptide and its use in stimulating apoptosis.
Preferably, the invention relates to a fragment of UNC5H2d spanning from amino acids 1 to 355 and its use in stimulating apoptosis.
Preferably, the invention relates to a fragment of UNC5H2d spanning from amino acids 26 to 355 and its use in stimulating apoptosis.
Preferably, the invention relates to a fragment of UNC5H2d consisting of the two immunoglobulin domains and the two thrombospondin domains of UNC5H2d and its use in stimulating apoptosis.
Preferably, the invention relates a fragment of UNC5H2d consisting of the two immunoglobulin domains of UNC5H2d and its use in stimulating apoptosis.
3) Furthermore, UCN5H2d may modulate angiogenesis and act as a pro-angiogenic factor. UNC5H2d prevents or disrupts netrin-1 binding to membrane bound UNC5H2 thereby promoting angiogenesis. UNC5H2d may therefore antagonize netrin-1's activity and stimulate angiogenesis. Thus, UNC5H2d activity can for example be confirmed in KO mice by measuring either accumulation of blood in the venous circulation and fluid in the pericardial activity, or by measuring peripheral resistance resulting from modified arterial vasculature, or by measuring capillaries thickness and branching in hindbrains, or by measuring filopodial extension from endothelial tip cells, or by characterizing intersegmental blood vessels (ISVs) trajectory phenotypes and more generally vessel-branching defects. A region of UNC5H2d which may be implicated in this stimulating activity corresponds to the extracellular part of membrane bound UNC5H2, i.e. spans from amino adds 1 to 355 of UNC5H2d. As such, the present invention also emcompasses the use of a fragment of UNC5H2d, i.e. spanning from amino adds 1 to 355 or from amino acids 26 to 355 of UNC5H2d in stimulating angiogenesis. Thus, the present invention also relates to the use of UNC5H2d or a fragment thereof in the treatment or prevention of cardiovascular diseases and/or hepatology related diseases. Different types of cardiovascular diseases and/or hepatology related diseases have already been disclosed supra.
Preferably, the invention relates to a soluble UNC5H2 and its use in stimulating angiogenesis.
Preferably, the invention relates to an UNC5H2d polypeptide and its use in stimulating angiogenesis.
Preferably, the invention relates to a fragment of UNC5H2d spanning from amino acids 1 to 355 and its use in stimulating angiogenesis.
Preferably, the invention relates to a fragment of UNC5H2d spanning from amino acids 26 to 355 and its use in stimulating angiogenesis.
Preferably, the invention relates to a fragment of UNC5H2d consisting of the two immunoglobulin domains and the two thrombospondin domains of UNC5H2d and its use in stimulating angiogenesis.
Preferably, the invention relates a fragment of UNC5H2d consisting of the two thrombospondin domains of UNC5H2d and its use in stimulating angiogenesis. Thrombosponding domains have been implicated in the inhibition of angiogenesis apoptosis and contributing to vascular homeostasis.

The following examples are intended for illustration purposes only, and should not be construed as limiting the scope of the invention in any way.

### EXAMPLES

### Example 1

1. Identification and Cloning of UNC5H2d

1.1 Introduction

The NCBI Data Bases were searched using human UNC5H2c (corresponding to NCBI's entry BAC57998) nucleotide sequence as query sequence. This lead to the identification of human UNC5H2a nucleotide sequence (corresponding to SwissProt entry Q8IZJ1 and NCBI's entries NP_{—}734465, AAM95701 and AY1264387). The UNC5H2a sequence was cloned by PCR from a lung cDNA library. The inserts of 5 clones were sequenced initially. It was found that 2 clones contained the longest UNC5H2 variant, i.e. UNC5H2c, spanning 17 exons. Two clones contained the UNC5H2a variant sequence (no exon 8). One clone was found which lacked exons 8 and 9 but was otherwise identical to UNC5H2c. The deletion of exons 8 and 9 leads to the removal of the putative transmembrane domain predicting a secreted form of this receptor. The sequence without exons 8 and 9 was named UNC5H2d.

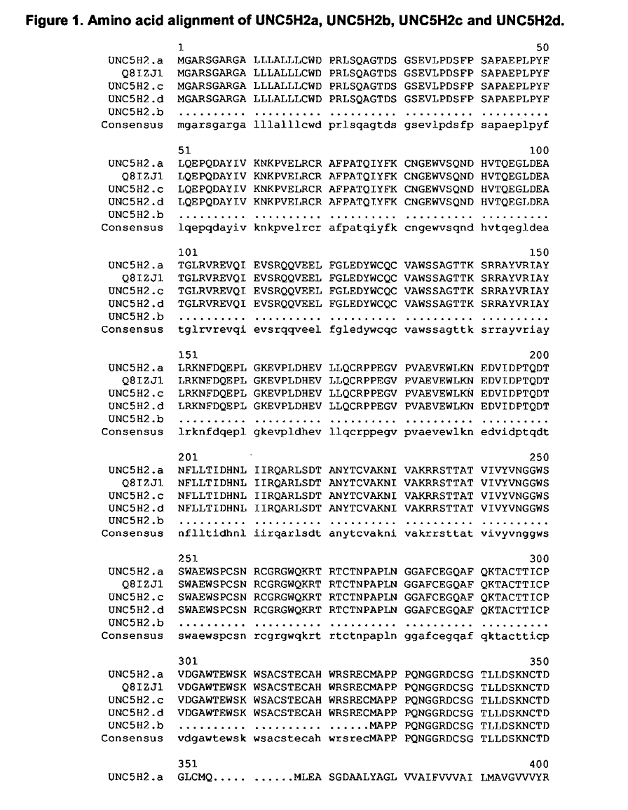

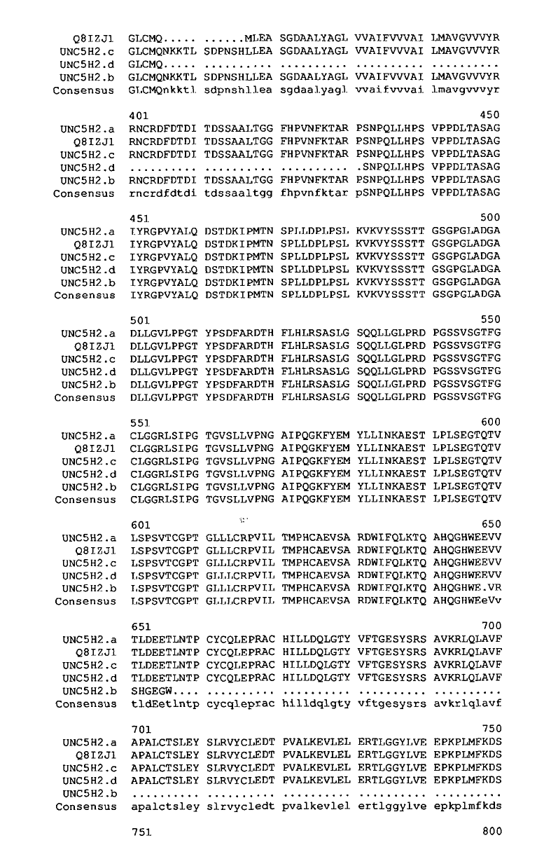

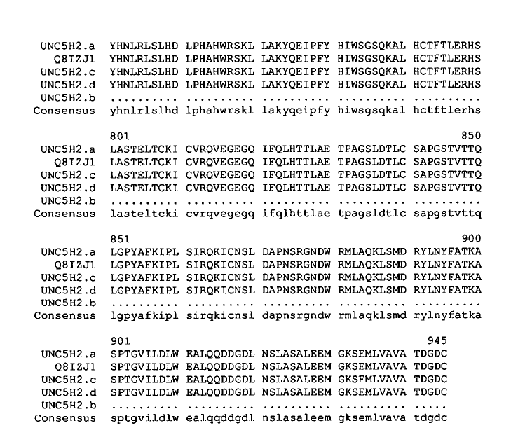

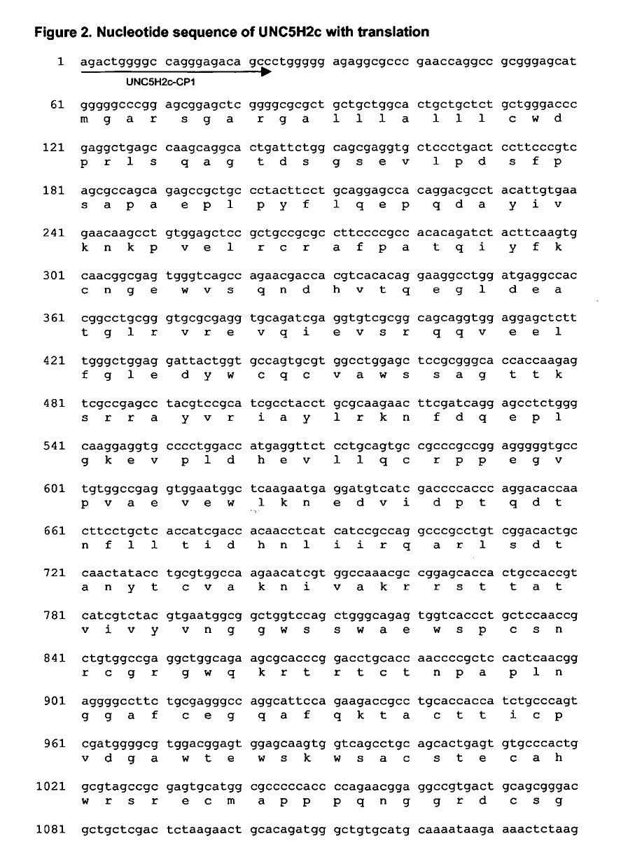

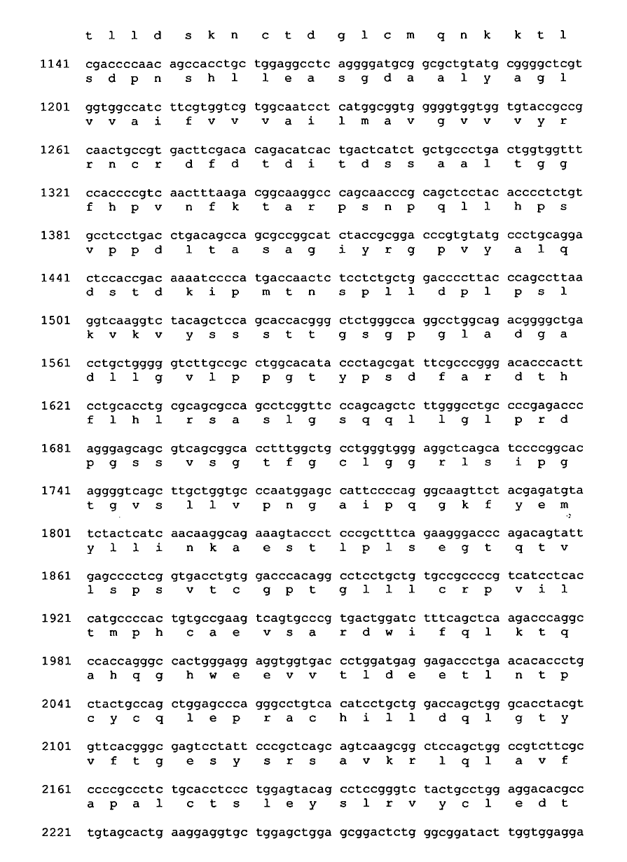

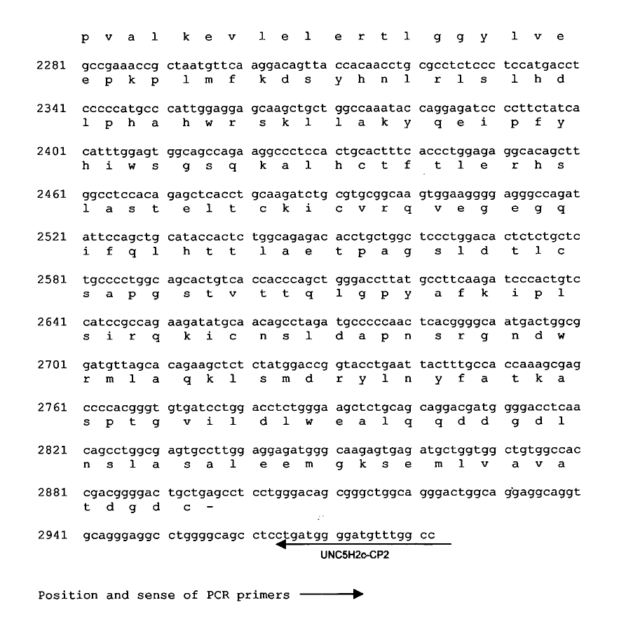

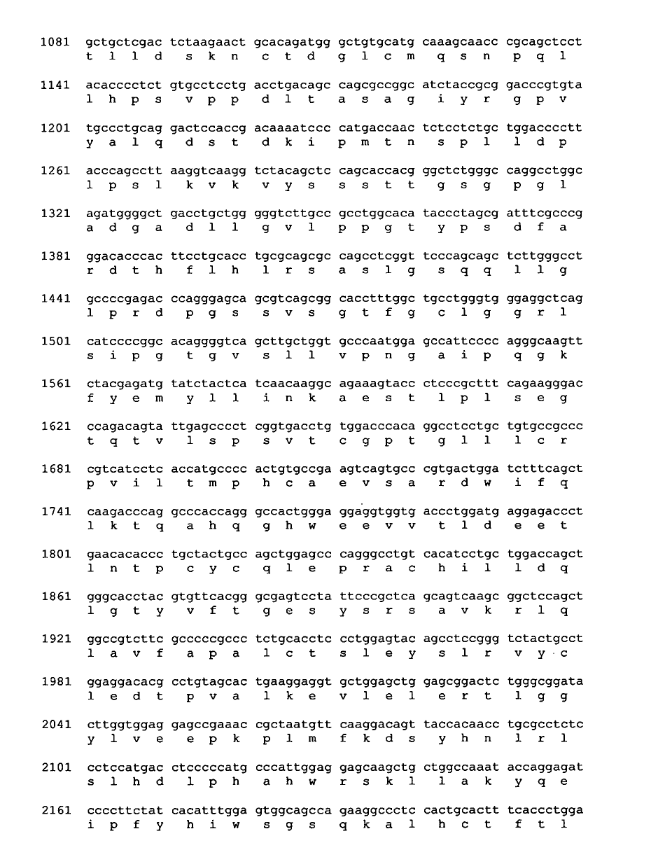

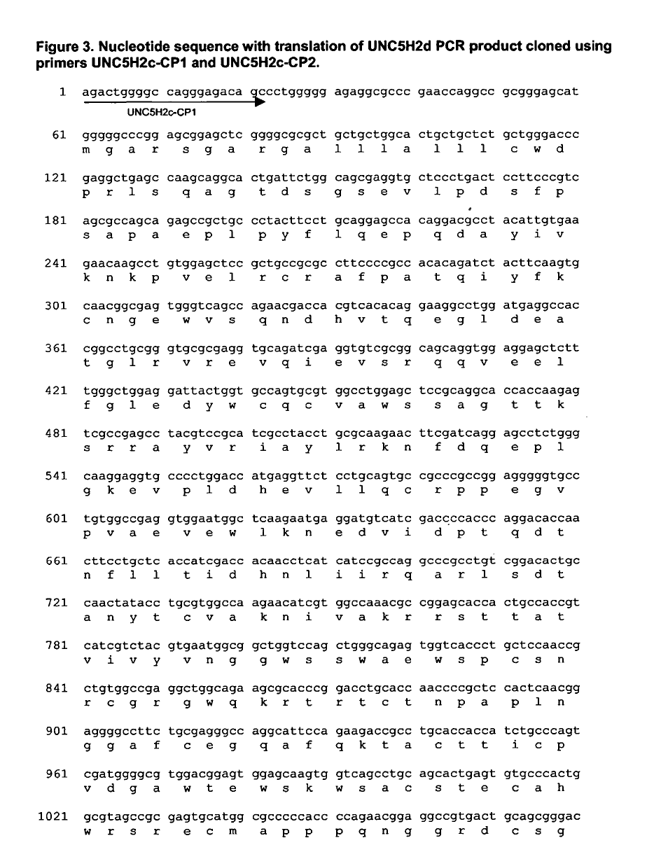

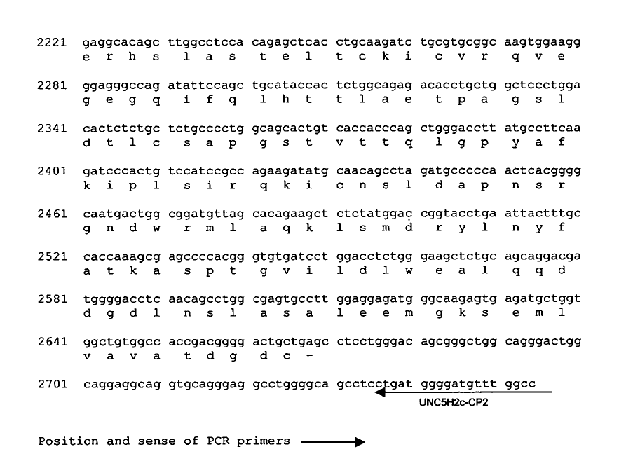

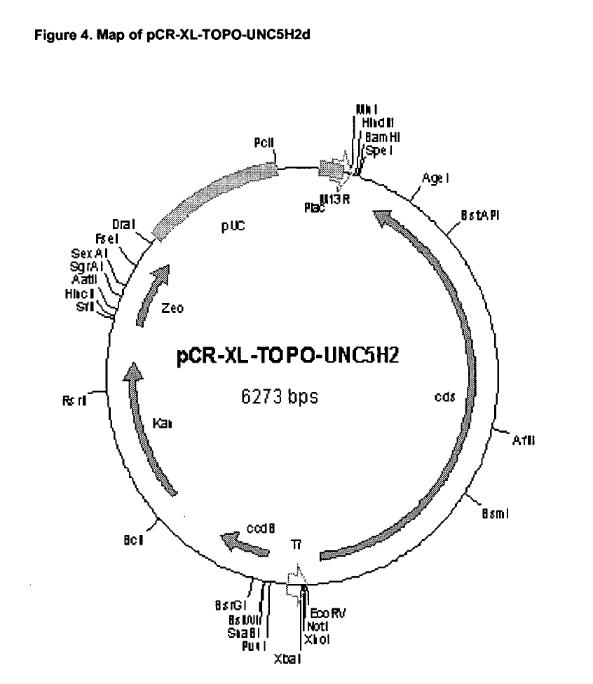

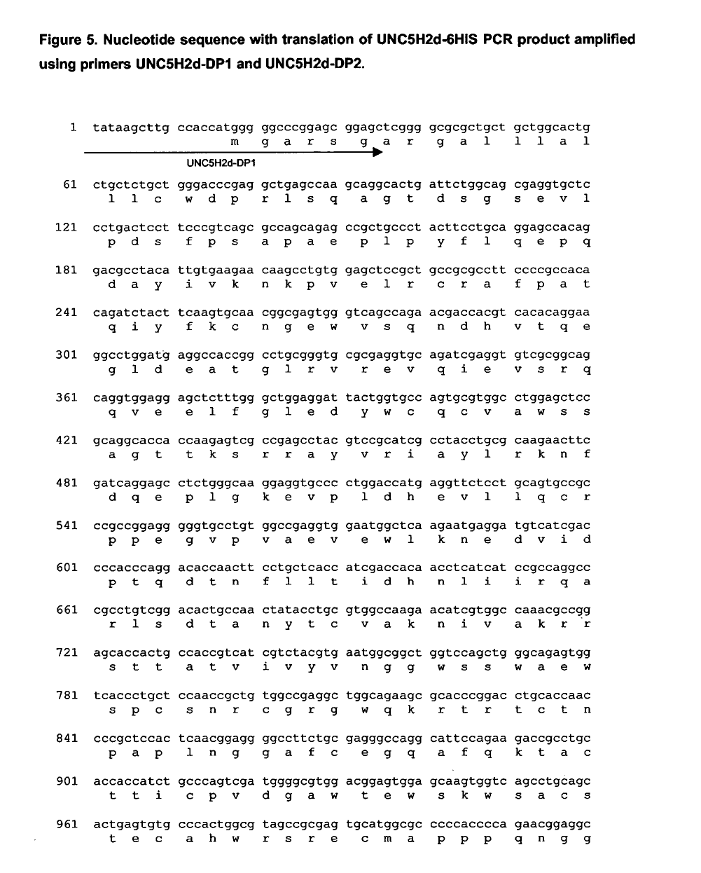

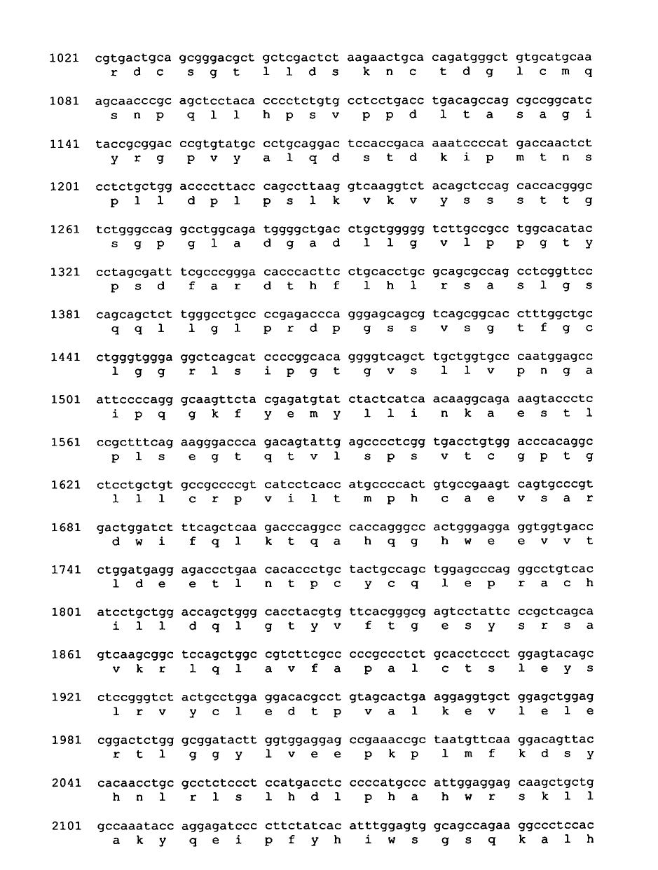

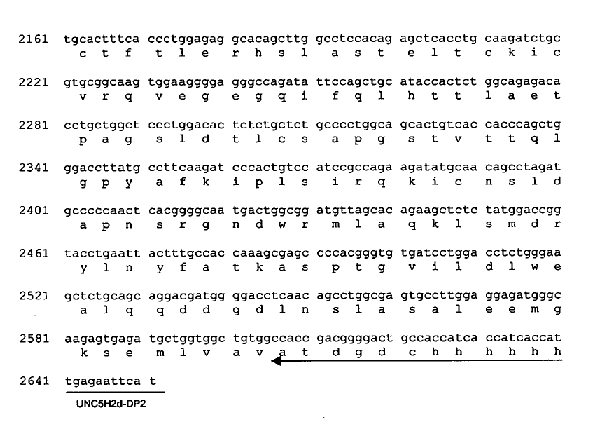

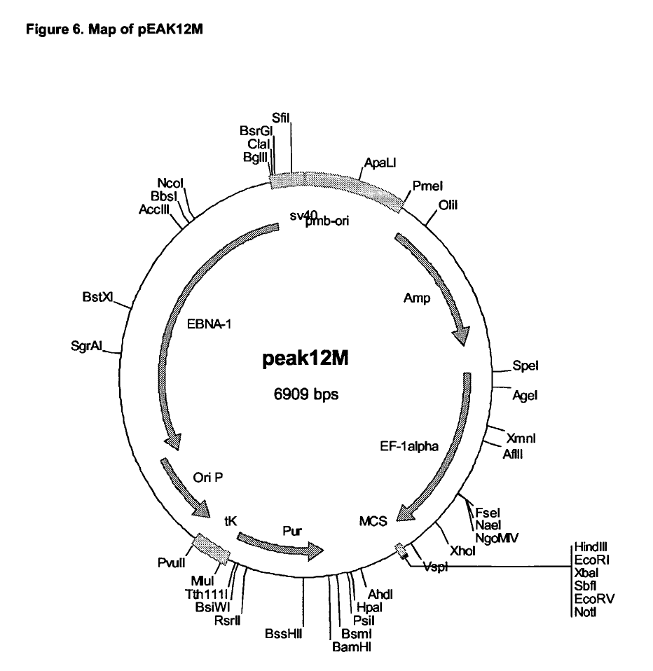

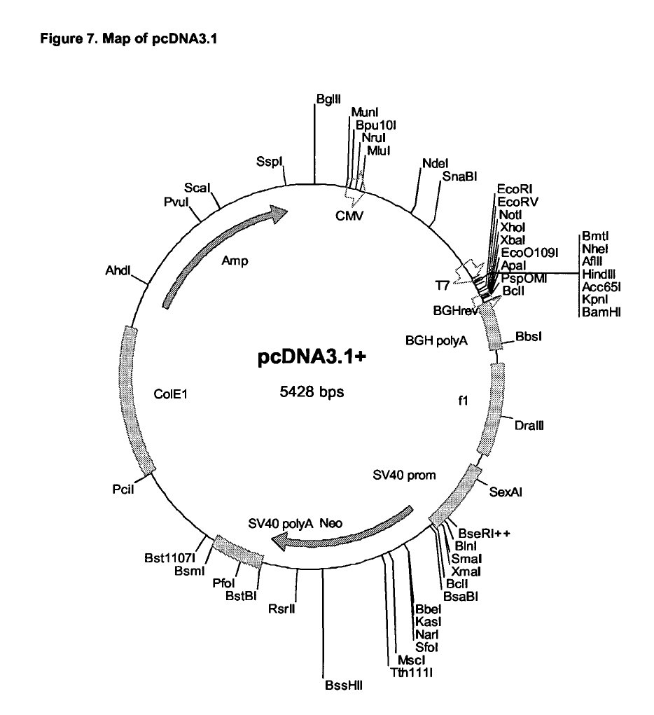

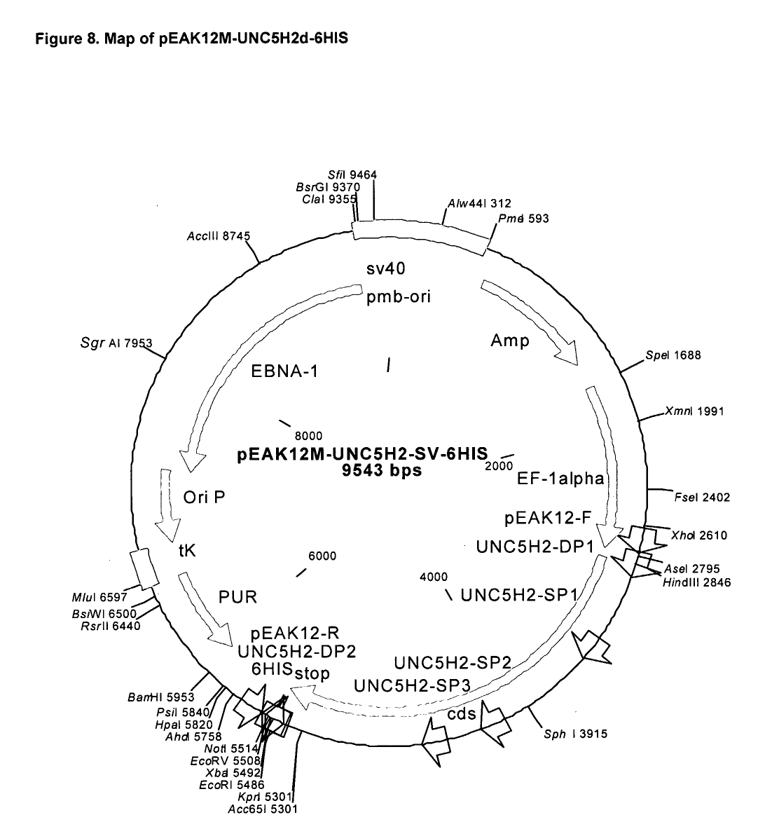

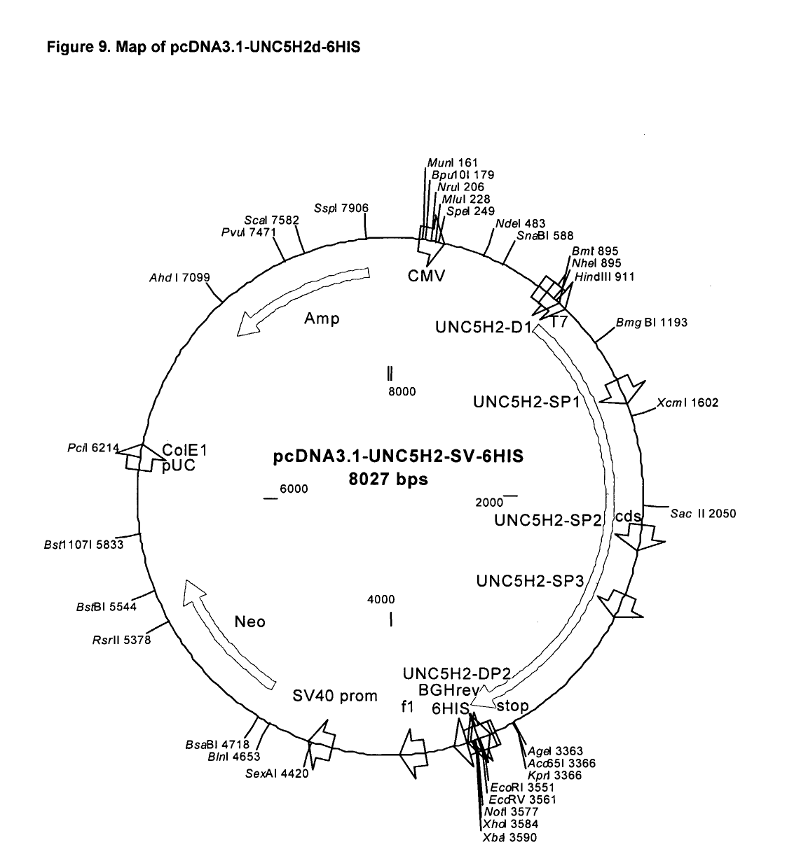

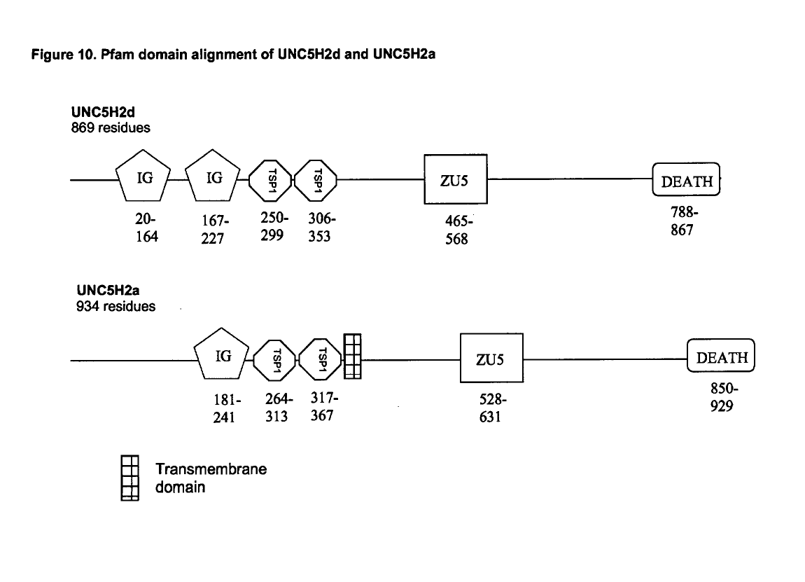

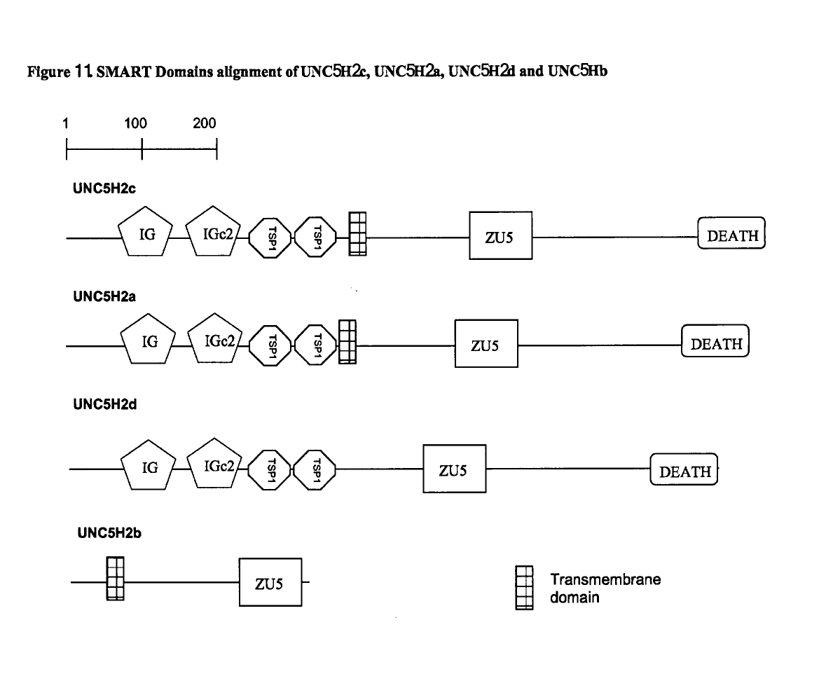

Hence, we have in this way identified a shorter, possibly soluble, splice variant of the UNC5H2c or UNC5H2a receptor. Because UNC5H2d does not contain the transmembrane domain it could represent a novel soluble secreted protein, which may act as UNC5H2 receptors antagonist in vivo. The alignment of the amino acid sequences of UNC5H2a, UNC5H2b, UNC5H2c and UNC5H2d is reported in FIG. 1.

1.2 Preparation of Human Lung cDNA

First strand cDNA was prepared from normal human lung total RNA (Clontech) using Superscript II Rnase H⁻ Reverse Transcriptase (Invitrogen) according to the manufacturer's protocol. One μl of oligo (dT)₁₅ primer (500 μg/ml, Promega), 2 μg human lung total RNA, 1 μl of 10 mM dNTPs (10 mM each of dATP, dGTP, dCTP and dTTP at neutral pH) and sterile distilled water to a final volume of 12 μl were combined in a 1.5 ml Eppendorf tube, heated to 65° C. for 5 min and then chilled on ice. The contents were collected by brief centrifugation and 4 μl of 5× First-Strand Buffer, 2 μl 0.1 M DTT, and 1 μl RnaseOUT (Recombinant Ribonuclease Inhibitor, 40 units/μl, Invitrogen) were added. The contents of the tube were mixed gently and incubated at 42° C. for 2 min, then 1 μl (200 units) of SuperScript II enzyme was added and mixed gently by pipeting. The mixture was incubated at 42° C. for 50 min and then inactivated by heating at 70° C. for 15 min. To remove RNA, 1 μl (2 units) of E. coli RNase H (Invitrogen) was added and the reaction mixture incubated at 37° C. for 20 min. The final 21 μl reaction mix was diluted to 200 μl with sterile water.

1.3 Gene Specific Cloning Primers for PCR

A pair of PCR primers having a length of between 18 and 25 bases were designed for amplifying the complete coding sequence of UNC5H2c cDNA using Primer Designer Software (Scientific & Educational Software, PO Box 72045, Durham, N.C. 27722-2045, USA). PCR primers were optimized to have a Tm dose to 55±10° C. and a GC content of 40-60%. Primers were selected which had high selectivity for the target sequence UNC5H2c (little or no non-specific priming).

1.4 PCR Amplification of UNC5H2c from Human Lung cDNA

A published sequence with GenBank accession number AY126473 corresponding to UNC5H2a cloned from a human lung cDNA library was identified through searching of public sequence databases which had 100% identity to UNC5H2c except for missing predicted exon 8. Gene-specific cloning primers (UNC5H2c-CP1 and UNC5H2c-CP2, FIG. 2 and FIG. 3 and Table 1) were designed to amplify a cDNA fragment of 2982 bp spanning the entire 2835 bp coding sequence of the UNC5H2c from human lung cDNA. Four identical PCRs was performed each in a final volume of 50 μl containing 1× Platinum® Taq High Fidelity PCR buffer, 2 mM MgSO₄, 200 μM dNTPs, 0.2 μM of each cloning primer, 2.5 units of Platinum® Taq DNA High Fidelity (Invitrogen) and 100 ng of human lung cDNA, using an MJ Research DNA Engine, programmed as follows: 94° C., 2 min; 35 cycles of 94° C., 30 s, 59° C., 30 s, 68° C., 3 min, (where 59° C. is the lowest Tm−5° C. and 3 min=1 min per kb of product); followed by 1 cycle at 68° C. for 7 min and a holding cycle at 4° C.

A 5 μl aliquot of each amplification product was visualized on a 0.8% agarose gel in 1× TAE buffer (Invitrogen) and a single PCR product was seen migrating at approximately the predicted molecular mass. The remaining PCR products were pooled and purified directly from the PCR mixture using the Wizard PCR Preps DNA Purification System (Promega) and eluted in 50 μl of sterile water. This product was subcloned directly.

1.5 Subcloning of PCR Products

PCR products were subcloned into the topoisomerase I modified cloning vector for long PCR products (pCR-XL-TOPO) using the TA cloning kit (Invitrogen Corporation) using the conditions specified by the manufacturer. Briefly, 4 μl of purified PCR product from the human lung cDNA amplification was incubated for 5 min at room temperature with 1 μl of pCR-XL-TOPO vector. 1 μl of 6× TOPO Cloning Stop Solution was then added and the reagents mixed, centrifuged briefly, and then placed on ice. The reaction mixture was transformed into E. coli strain TOP10 (Invitrogen) as follows: a 50 μl aliquot of One Shot TOP10 electrocompetent cells was thawed on ice and 2 μl of the TOPO reaction was added. The mixture was transferred to a chilled 0.1 cm electroporation cuvette and the cells electroporated using a BioRad Gene-Pulser™ according to the manufacturer's recommended protocol. SOC media (450 μl) which had been warmed to room temperature was added immediately after electroporation and the solution transferred to a 15 ml snap-cap tube and incubated, with shaking (220 rpm) for 1 h at 37° C. Aliquots of the transformation mixture (50 μl, 100 μl and 200 μl) were then plated on L-broth (LB) plates containing kanamycin (40 μg/ml) and incubated overnight at 37° C. Kanamycin resistant colonies containing cDNA inserts were identified by colony PCR.

1.6 Colony PCR

Colonies were inoculated into 50 μl sterile water using a sterile toothpick. A 10 μl aliquot of the inoculum was then subjected to PCR in a total reaction volume of 20 μl as described above, except the primers used were M13R and T7. The cycling conditions were as follows: 94° C., 2 min; 30 cycles of 94° C., 30 s, 47° C., 30 s and 72° C. for 3.5 min. Samples were then maintained at 4° C. (holding cycle) before further analysis.

PCR reaction products were analyzed on 1% agarose gels in 1× TAE buffer. Colonies which gave the expected PCR product size (approx. 2982 bp cDNA+186 bp due to the multiple cloning site or MCS in the vector) were grown up overnight at 37° C. in 5 ml L-Broth (LB) containing kanamycin (40 μg/ml), with shaking (220 rpm) at 37° C.

1.7 Plasmid DNA Preparation and Sequencing

Miniprep plasmid DNA was prepared from 5 ml cultures using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer. Plasmid DNA (200-500 ng) was subjected to DNA sequencing with T7, M13R and UNC5H2c gene-specific sequencing primers (UNC5H2c-SP-1 to -6) using the BigDyeTerminator system (Applied Biosystems cat. no. 4390246) according to the manufacturer's instructions. The primer sequences are listed in Table 1. Sequencing reactions were purified using Dye-Ex columns (Qiagen) or Montage SEQ 96 cleanup plates (Millipore cat. no. LSKS09624) then analyzed on an Applied Biosystems 3700 sequencer.

Sequence analysis identified one clone which contained a 100% match to UNC5H2c sequence but was missing exons 8 and 9. The sequence of this cloned cDNA fragment (UNC5H2d) is shown in FIG. 3. The plasmid map of the cloned PCR product (pCR-XL-TOPO-UNC5H2d) (plasmid ID. 13855) is shown in FIG. 4.

TABLE 1
UNC5H2c cloning and sequencing primers

| | |
|---|---|
| Primer | Sequence (5′-3′) |

| | |
|---|---|
| UNC5H2c-CP1 | AGA CTG GGG CCA GGG AGA CA |
| UNC5H2c-CP2 | GGC CAA ACA TCC CCA TCA GG |
| UNC5H2c-SP1 | GAG GTG GAA TGG CTC AAG AA |
| UNC5H2c-SP2 | CTC GTG GTG GCC ATC TTC GT |
| UNC5H2c-SP3 | TCT TGC CGC CTG GCA CAT AC |
| UNC5H2c-SP4 | ACT GTG CCG AAG TCA GTG |
| UNC5H2c-SP5 | GGA GTG GAG CAA GTG GTC AG |
| UNC5H2c-SP6 | TCA GGT CAG GAG GCA CAG AG |
| T7 primer | TAA TAC GAC TCA CTA TAG G |
| M13R primer | CAG GAA ACA GCT ATG ACC |

2. Construction of Mammalian Cell Expression Vectors for UNC5H2d
2.1 Generation of Ligation-Ready UNC5H2d ORF Fused to an In-Frame 6HIS Tag Sequence

The first stage of the cloning process involved a PCR reaction to generate the ORF of UNC5H2d flanked at the 5′ end by a HindIII restriction endonuclease recognition site and Kozak sequence, and flanked at the 3′ end by a sequence encoding an in-frame 6 histidine (6HIS) tag, a stop codon and an EcoRI restriction endonuclease recognition site. The UNC5H2d ORF sequence was amplified from the plasmid pCR-XL-UNC5H2d (plasmid ID 13855) using PCR amplification primers UNC5H2d-DP1 and UNC5H2d-DP2 (Table 2 and FIG. 5). Forward amplification primer UNC5H2d-DP1 was designed to add an HindIII restriction endonuclease recognition site (AAGCTT) and a Kozak sequence (GCCACC) immediately upstream of the UNC5H2d methionine start codon. Reverse amplification primer UNC5H2d-DP2 was designed to add a 6HIS tag (CAC CAT CAC CAT CAC CAT), a stop codon (TGA) and an EcoRI (GAATTC) restriction endonuclease recognition site at the 3′ end of the UNC5H2d coding sequence.

The PCR was performed in a final volume of 50 μl containing 1× Platinum® Pfx PCR buffer, 1 mM MgSO₄, 300 μM dNTPs, 0.3 μM UNC5H2d-DP1, 0.3 μM UNC5H2d-DP2, 1.25 units of Platinum® Pfx DNA polymerase (Invitrogen), 170 ng of plasmid 13855 DNA, and either 0×, 0.5×, 1×, or 2× PCRₓ Enhancer solution (Invitrogen). Cycling was performed using an MJ Research DNA Engine, programmed as follows: 94° C., 5 min; 30 cycles of 94° C., 15 sec, 68° C., 3 min; followed by 1 cycle at 68° C. for 7 min and a holding cycle at 4° C.

All 50 μl of each amplification reaction was visualized on 0.8% agarose gel in 1× TAE buffer (Invitrogen) and a single PCR product was seen migrating at the predicted molecular mass (2651 bp) in all samples. The PCR products were excised from the gel and pooled into 2 fractions. The DNA was extracted using the Wizard PCR Preps DNA Purification System (Promega) and eluted in a 50 μl of sterile water. The purified products were combined and a 5 μl aliquot visualized on an agarose gel to estimate the concentration.

All of the purified PCR products (approximately 1.8 μg of DNA in 90 μl) were digested using restriction endonucleases HindIII and EcoRI (New England Biolabs, NEB) in a final reaction volume of 110 μl containing 11 μl 10× EcoRI digestion buffer (NEB), 50 units EcoRI endonuclease, and 50 units HindIII endonuclease. The digestion was carried out at 37° C. for 1 hour. The digestion products were visualised on a 0.8% agarose gel in 1× TAE buffer. The 2649 bp products were excised from the gel and the DNA extracted using the Qiaquick Gel Extraction kit (Qiagen). The DNA was eluted in 30 μl EB buffer (10 mM Tris-Cl, pH 8.5). A 1 μl aliquot of the purified product was visualised on an agarose gel and the concentration estimated to be approximately 50 ng/μl.

2.2 Generation of Ligation-Ready Linear Expression Vector Fragments

The empty expression vectors pEAK12M (plasmid ID 13040, FIG. 6) and pcDNA3.1 (Invitrogen, FIG. 7) were digested with the HindIII and EcoRI restiction endonucleases in sequential reactions. Five μg of each empty vector was digested in a final reaction volume of 100 μl containing either 10 μl 10× EcoRI digestion buffer (NEB) and 60 units EcoRI endonuclease (NEB), or 10 μl 10× NEB restriction buffer 2 and 60 units HindIII endonuclease (NEB). The digestions were carried out at 37° C. for 1 hour. A 5 μl aliquot of each digestion product was visualised on an agarose gel to confirm that the vector had been linearised. The products were purified using the Wizard DNA Clean-up system (Promega) and eluted in 50 μl water. Each product was then digested with the second restriction endonuclease in a final reaction volume of 70 μl containing either 7 μl 10× EcoRI digestion buffer (NEB) and 60 units EcoRI endonuclease (NEB), or 7 μl 10× NEB restriction buffer 2 and 60 units HindIII endonuclease (NEB). The digestions were carried out at 37° C. for 1 hour. All digestion products were visualised on 0.8% agarose gel in 1× TAE buffer and the linear vector fragments of expected molecular mass (6903 bp for the linear pEAK12M vector and 5387 bp for the linear pcDNA3.1 vector) were purified using the Qiaquick Gel Extraction kit (Qiagen) as above. Each product was eluted in 30 μl EB buffer (10 mM Tris-Cl, pH 8.5).

Each 30 μl linear vector sample was combined with 2 units of Calf Intestinal Alkaline Phosphatase (CIAP, Roche) and 5 μl 10× CIAP buffer (Roche) in a final volume of 50 μl. The reaction mixture was incubated at 37° C. for 15 min and then at 50° C. for 15 min. A further 2 units of CIAP were added to each reaction mixture and the 37° C. and 50° C. incubations repeated. The two pEAK12M samples and the two pcDNA3.1 samples were then combined and purified using the Wizard DNA Clean-up system (Promega). The products were eluted in 50 μl water. A 2 μl aliquot of each elute was visualised on an agarose gel and the concentration of the DNA estimated to be approximately 20 ng/μl for the linear pEAK12M vector sample and approximately 25 ng/μl for the linear pcDNA3.1 vector sample.

2.3 Subcloning of UNC5H2d ORF into Expression Vectors pEAK12M and pcDNA3.1

The digested PCR product from Section 2.1 was ligated into 100 ng of each linear vector (prepared in Section 2.2) in the ratio of approximately ‘3 insert: 1 vector’ according to the formula:
[(vector (ng)×size of insert (bp))/size of vector (bp)]×3=insert (ng)

The pEAK12M ligation reaction was performed in a final volume of 20 μl containing 2 μl 10× ligase buffer (NEB), 400 units T4 DNA ligase (NEB), 100 ng linearized pEAK12M vector and 115 ng PCR product. The pcDNA3.1 ligation reaction was performed in a final volume of 20 μl containing 2 μl 10× ligase buffer (NEB), 400 units T4 DNA ligase (NEB), 100 ng linearized pcDNA3.1 vector and 150 ng PCR product. Each ligation reaction mixture was incubated at 23° C. for 40 min.

2 μl of each ligation reaction were transformed into E. coli strain TOP10 (Invitrogen) as follows: a 50 μl aliquot of One Shot TOP10 cells was thawed on ice and 2 μl of ligation reaction mixture was added. The mixture was incubated for 15 min on ice and then heat shocked by incubation at 42° C. for exactly 30 s. Samples were returned to ice and 250 μl of warm SOC media (room temperature) was added. Samples were incubated with shaking (220 rpm) for 1 h at 37° C. 10 μl, 50 μl and 200 μl aliquots of each transformation mixture was plated on L-broth (LB) plates containing ampicillin (100 μg/ml) and incubated overnight at 37° C. Ten of the resultant ampicillin resistant colonies from each ligation reaction were grown up overnight at 37° C. in 5 ml L-Broth (LB) containing ampicillin (100 μg/ml), with shaking at 220 rpm at 37° C.

2.4 Plasmid DNA Preparation and Sequencing

Miniprep plasmid DNA was prepared from 5 ml cultures using a Qiaprep Turbo 9600 robotic system (Qiagen) according to the manufacturer's instructions. Plasmid DNA was eluted in 100 μl of sterile water. Plasmid DNA (200-500 ng) was subjected to DNA sequencing with vector-specific primers using the BigDyeTerminator system (Applied Biosystems cat. no. 4390246) according to the manufacturer's instructions. The pEAK12M constructs were sequenced using the sequencing primers pEAK12-F and pEAK12-R, and the pcDNA3.1 constructs were sequenced using the primers T7 and BGHreverse. The primer sequences are shown in Table 2. Sequencing reactions were purified using Dye-Ex columns (Qiagen) or Montage SEQ 96 cleanup plates (Millipore cat. no. LSKS09624) then analyzed on an Applied Biosystems 3700 sequencer. Clones which were identified as containing the expected insert were subjected to sequencing using internal gene-specific primers UNC5H2d-SP1, UNC5H2d-SP2 and UNC5H2d-SP3 (Table 2).

Sequence analysis identified clones which contained the UNC5H2d-6HIS insert in vector pEAK12M (pEAK12M-UNC5H2d-6HIS, plasmid ID 13999) and in vector pcDNA3.1 (pcDNA3.1-UNC5H2d-6HIS, plasmid ID 14000). The sequence of the cloned cDNA fragment is shown in FIG. 5. The plasmid maps of the cloned PCR products are shown in FIGS. 8 and 9.

CsCl gradient purified maxi-prep DNA was prepared from a 500 ml culture of the sequence verified clone pEAK12M-UNC5H2d-6HIS (plasmid ID number 13999) using the method described by Sambrook J. et al., 1989 (Molecular Cloning, a Laboratory Manual, 2^{nd} edition, Cold Spring Harbor Laboratory Press). Plasmid DNA was resuspended at a concentration of 1 μg/μl in sterile water (or 10 mM Tris-HCl pH 8.5) and stored at −20° C.

Endotoxin-free maxi-prep DNA was prepared from a 500 ml culture of the sequence verified clone pcDNA3.1-UNC5H2d (plasmid ID 14000) using the EndoFree Plasmid Mega kit (Qiagen) according to the manufacturers instructions. Plasmid DNA was resuspended in 500 μl of endotoxin-free TE buffer (10 mM Tris-Cl, pH 8.0, 1 mM EDTA).

TABLE 2

UNC5H2d-6HIS PCR cloning and sequencing
primers:

| | |
|---|---|
| Primer | Sequence (5′-3′) |

| | |
|---|---|
| UNC5H2d-DP1 | TAT [symbol] GCC ACC ATG GGG GCC CGG AGC |
| | GGA G |
| UNC5H2d-DP2 | ATG [symbol] T CAA TGG TGA TGG TGA TGG TGG |
| | CAG TCC CCG TCG GTG G |
| UNC5H2d-SP1 | GAG GTG GAA TGG CTC AAG AA |
| UNC5H2d-SP2 | TCT TGC CGC CTG GCA CAT AC |
| UNC5H2d-SP3 | ACT GTG CCG AAG TCA GTG |
| pEAK12-F | GCC AGC TTG GCA CTT GAT GT |
| pEAK12-R | GAT GGA GGT GGA CGT GTC AG |
| T7 | TAA TAC GAC TCA CTA TAG G |
| BGH reverse | TAG AAG GCA CAG TCG AGG |

Underlined sequence = Kozak sequence
Bold = Stop codon
Italic sequence = His tag
Bold and italic = Restriction endonuclease recognition site

### Example 2

### Identification and Description of UNC5H2 Domains

2.1 Identification

Bioinformatic tools were used to identify the putative domains of the splice variant UNC5H2d. All tools identify domains in query sequences. SMART (results shown in FIG. 11), BLOCKS (http://blocks.fhcrc.org/blocks/), and Pfam (Protein FAMilies database of alignments and HMMs, http://www.sanger.ac.uk/Software/Pfam/, results shown in FIG. 10) identified the following domains, as indicated by the following tables (tables 2 to 6):

1) Pfam Hits

TABLE 3
Trusted matches - domains scoring higher
than the gathering threshold

| Domain | Start | End | Bits | Evalue | Alignment | Mode |
|---|---|---|---|---|---|---|
| ig | 167 | 227 | 30.00 | 4.8e−6 | Align | Is |
| tsp 1 | 250 | 299 | 34.30 | 2.5e−07 | Align | Is |
| tsp 1 | 306 | 353 | 20.90 | 0.0004 | Align | Is |
| ZU5 | 465 | 568 | 202.00 | 8.2e−58 | Align | Is |
| death | 788 | 867 | 74.10 | 2.5e−19 | Align | Is |

TABLE 4
Matches to Pfam-B

| Domain | Start | End | Alignment |
|---|---|---|---|
| Pfam-B 9659; | 20 | 164 | Align |

TABLE 5
Potential matches - Domains with Evalues above the cutoff

| Domain | Start | End | Bits | Evalue | Alignment | Mode |
|---|---|---|---|---|---|---|
| metalthio | 276 | 350 | −12.30 | 0.77 | Align | Is |

TABLE 6
Other regions

| Type | Source | Start | End |
|---|---|---|---|
| low_complexity | seg | 2 | 17 |
| low_complexity | seg | 417 | 431 |
| low_complexity | seg | 452 | 461 |

2) BLOCKS Hits
Query=UNC5H2d:
Size=869 Amino Acids
Blocks Searched=11182
Alignments Done=10004755
Cutoff combined expected value for hits=1

Cutoff block expected value for repeats/other=1

TABLE 7

| Combined | | | | |
|---|---|---|---|---|
| Family | | Strand | Blocks | E-value |
| IPB000906 | ZU5 domain | 1 | 1 of 8 | 1e−06 |
| IPB002861 | Reeler domain | 1 | 2 of 3 | 2.96−06 |
| IPB001862 | Membrane attack | 1 | 1 of 6 | 0.0073 |
| | complex components/ | | | |
| PR01303 | Plasmodium | 1 | 1 of 6 | 0.093 |
| | circumsporozoite protein | | | |
| IPB003884 | Factor I membrane | 1 | 2 of 6 | 0.67 |
| | attack complex | | | |
| IPB001307 | Rhodanese signatures | 1 | 1 of 7 | 0.98 |

2.2 Description of the Domains

The Death domain motif is found in 2 isoforms from this gene. 35 other genes in the database also contain this motif. [InterPro annotation] The death domain (FAS/TNF cytosolic interaction domain) has first been described as a region in the cytoplasmic tail of the 75 Kd TNF receptor (TNFR-1) (see [PROSITEDOC:PDOC00561]) which is involved in TNF-mediated cell death signaling. A corresponding region is found in the cytoplasmic tail of FAS/APO1 another surface receptor inducing apoptotic cell death. This region mediates self-association of these receptors, thus giving the signal to downstream events leading to apoptosis. Subsequently, a number of other proteins have been found to interact with the cytoplasmic part of either FAS or the TNF receptor in the region of the death domain. Overexpression of these proteins usually leads to cell death. By profile analysis, it has been shown that a number of other proteins contain regions with significant similarity to the death domain. Interestingly, several of these proteins also work in the context of cell death signaling. In most of these proteins, the death domain is located at the extreme C-terminus. Exceptions are ankyrin, MyD88 and pelle, all proteins probably not directly involved in cell death signaling. In the case of ankyrin, the isoform 2.1 is a splice variant which has the death domain located at the C-terminus.

The Immunoglobulin/major histocompatibility complex motif is seen in the product of this gene. 506 other genes in the database also contain this motif. [InterPro annotation] The basic structure of immunoglobulin (Ig) molecules is a tetramer of two light chains and two heavy chains linked by disulfide bonds. There are two types of light chains: kappa and lambda, each composed of a constant domain (CL) and a variable domain (VL). There are five types of heavy chains: alpha, delta, epsilon, gamma and mu, all consisting of a variable domain (VH) and three (in alpha, delta and gamma) or four (in epsilon and mu) constant domains (CH1 to CH4). The major histocompatibility complex (MHC) molecules are made of two chains. In class I the alpha chain is composed of three extracellular domains, a transmembrane region and a cytoplasmic tail. The beta chain (beta-2-microglobulin) is composed of a single extracellular domain. In class II, both the alpha and the beta chains are composed of two extracellular domains, a transmembrane region and a cytoplasmic tail. It is known that the Ig constant chain domains and a single extracellular domain in each type of MHC chains are related. These homologous domains are approximately one hundred amino acids long and include a conserved intradomain disulfide bond. Members of the immunoglobulin superfamily are found in hundreds of proteins of different functions. Examples include antibodies, the giant muscle kinase titin and receptor tyrosine kinases. Immunoglobulin-like domains may be involved in protein-protein and protein-ligand interactions. The Pfam alignments do not include the first and last strand of the immunoglobulin-like domain. Some of the proteins in this group are responsible for the molecular basis of the blood group antigens, surface markers on the outside of the red blood cell membrane. Most of these markers are proteins, but some are carbohydrates attached to lipids or proteins [Reid M. E., Lomas-Francis C. The Blood Group Antigen FactsBook Academic Press, London/San Diego, (1997)]. Lutheran blood group glycoprotein (B-CAM cell surface glycoprotein) (Auberger B antigen) (F8/G253 antigen) belongs to the Lutheran blood group system and is associated with Lu(a/b), Au(a/b), LU6 to LU20 antigens. Igc2 subfamily is the C-2-type and includes glycoproteins and other unrelated proteins with immunoglobin domains e.g. carcinoembryonic antigens and fibroblast growth factor receptors. The IG subfamily includes killer cell inhibitory receptor, as well as other cell surface receptors containing an immunoglobin domain.

The Thrombospondin, type I motif is seen in the product of this gene. 59 other genes in the database also contain this motif. [InterPro annotation] This repeat was first described in 1986 by Lawler & Hynes. It was found in the thrombospondin protein where it is repeated 3 times. Now a number of proteins involved in the complement pathway (properdin, C6, C7, C8A, C8B, C9) as well as extracellular matrix protein like mindin, F-spondin SCO-spondin and even the circumsporozoite surface protein 2 and TRAP proteins of Plasmodium, ADAMTS and G protein-coupled receptors, such as the brain-specific angiogenesis inhibitors contain one or more instance of this repeat. The domains have a number of functions, including effects on cell attachment, cell-cell interaction, motility, proliferation, the activities of extracellular proteases, and inhibition of angiogenesis apoptosis, contributing to vascular homeostasis. The intron-exon organisation of the properdin gene confirms the hypothesis that the repeat might have evolved by a process involving exon shuffling.

The domain is ˜60 amino acid residues in length and is characterised by a highly conserved W-S-X-W motif and six cysteine residues. A study of the structure of properdin indicates that the TSP1 repeat contains two amphipathic turn regions and a hydrophilic beta-strand.

The ZU5 domain motif is found in 3 isoforms from this gene. 8 other genes/TJP1, ANK3, ANK1, UNC5D, ZU5, ZU5.1, ANK2, UNC5C/in the database also contain this motif.

[InterPro annotation] This is a domain of unknown function, present in zonula occludens 1 (ZO-1 or tight junction protein 1)) and Unc5-like netrin receptors. It is also found in different variants of ankyrin, which are responsible for attaching integral membrane proteins to cytoskeletal elements.

BLOCKS: Reeler Region:

[InterPro annotation] Extracellular matrix (ECM) proteins play an important role in early cortical development, specifically in the formation of neural connections and in controlling the cyto-architecture of the central nervous system. The product of the reeler gene in mouse is reelin, a large extracellular protein secreted by pioneer neurons that coordinates cell positioning during neurodevelopment [1]. F-spondin and mindin are a family of matrix-attached adhesion molecules that share structural similarities and overlapping domains of expression. Both F-spondin and mindin promote adhesion and outgrowth of hippocampal embryonic neurons and bind to a putative receptor(s) expressed on both hippocampal and sensory neurons [2]. This domain of unknown function is found at the N terminus of reelin and F-spondin (see http://www.bork.embl-heidelberg.de/Modules/07-matrix.gif).

BLOCKS: Plasmodium Circumsporozoite Protein Signature

[PRINTS ANNOTATION]The circumsporozoite (CS) protein is the most prominant surface antigen on the sporozoite of the malaria parasite, Plasmodium spp. The sporozoite is the infectious stage of the Plasmodium life cycle, the form in which malaria is passed from the mosquito vector to the mammalian host. Antibodies to this protein are used in the field to detect exposure to malaria and it is a target for several vaccines. The sequence of the CS protein consists of head and tail regions, which are largely conserved, and a large set of low-complexity repeats, which are variant across strain and species. The C-terminal region is probably used for anchoring the protein to the cell membrane, while the central repeat sequences would be the surface antigen of the organism. The repeats, which encode the immunodominant epitope of the CS protein, diverge more rapidly than the remainder of the gene. It is thought that the maintenance and evolution of the repeats is achieved via a mechanism that acts not at the protein level but, rather, directly on the DNA sequence.

CRCMSPRZOITE is a 6-element fingerprint that provides a signature for Plasmodium circumsporozoite proteins. The fingerprint was derived from an initial alignment of 7 sequences: the motifs were drawn from the conserved N— and C-terminal regions of the alignment, flanking the central repeats—motif 1 spans the putative signal sequence. Two iterations on SPTR37_{—}10f were required to reach convergence, at which point a true set comprising 49 sequences was identified.

BLOCKS: Factor I Membrane Attack Complex

[InterPro Annotation] This domain is found in complement component proteins, complement component factor 1 and agrin. Factor I is responsible for cleaving alpha chains of C4B and C3B in the presence of the cofactors C4-binding protein and factor H respectively. Agrin is a component of the basal lamina that causes the aggregation of acetylcholine receptors and acetylcholine-esterase on the surface of muscle fibers of the neuromuscular junction.

BLOCKS: Rhodanese Signatures

Thiosulfate sulfurtransferase (EC: 2.8.1.1) is an enzyme which catalyzes the transfer of the sulfane atom of thiosulfate to cyanide, to form sulfite and thiocyanate. In vertebrates, rhodanese is a mitochondrial enzyme that is involved in forming iron-sulfur complexes and cyanide detoxification. A cysteine residue takes part in the catalytic mechanism [1, 2]. Some bacterial proteins may also express sulfotransferase activity. These include, sseA from Mycobacterium leprae and E. coli, Azotobacter vinelandii rhdA, Saccharopolyspora erythraea cysA [3] and Synechococcus strain PCC 7942 rhdA [4]. Rhodanese, a sulfurtransferase involved in cyanide detoxification (see IPR001307) shares evolutionary relationship with a large family of proteins [1], including

Cdc25 phosphatase catalytic domain.

non-catalytic domains of eukaryotic dual-specificity MAPK-phosphatases.

non-catalytic domains of yeast PTP-type MAPK-phosphatases.

non-catalytic domains of yeast Ubp4, Ubp5, Ubp7.

non-catalytic domains of mammalian Ubp-Y.

Drosophila heat shock protein HSP-67BB.

several bacterial cold-shock and phage shock proteins.

plant senescence associated proteins.

catalytic and non-catalytic domains of rhodanese (see IPR001307).

Rhodanese has an internal duplication. This domain is found as a single copy in other proteins, including phosphatases and ubiquitin C-terminal hydrolases [2].

[INTERPRO ANNOTATION] Metallothioneins (MT) are small proteins that bind heavy metals, such as zinc, copper, cadmium, nickel, etc. They have a high content of cysteine residues that bind the metal ions through clusters of thiolate bonds [MEDLINE:89118264], PUB00001490. An empirical classification into three classes has been proposed by Fowler and coworkers PUB00001490 and Kojima PUB00001490. Members of class I are defined to include polypeptides related in the positions of their cysteines to equine MT-1B, and include mammalian MTs as well as from crustaceans and molluscs. Class II groups MTs from a variety of species, including sea urchins, fungi, insects and cyanobacteria. Class III MTs are atypical polypeptides composed of gamma-glutamylcysteinyl units [MEDLINE:88029881].

This original classification system has been found to be limited, in the sense that it does not allow clear differentiation of patterns of structural similarities, either between or within classes. Consequently, all class I and class II MTs (the proteinaceous sequences) have now been grouped into families of phylogenetically-related and thus alignable sequences. This system subdivides the MT superfamily into families, subfamilies, subgroups, and isolated isoforms and alleles.

The metallothionein superfamily comprises all polypeptides that resemble equine renal metallothionein in several respects [MEDLINE:88029881]: e.g., low molecular weight; high metal content; amino acid composition with high Cys and low aromatic residue content; unique sequence with characteristic distribution of cysteines, and spectroscopic manifestations indicative of metal thiolate clusters. A MT family subsumes MTs that share particular sequence-specific features and are thought to be evolutionarily related. The inclusion of a MT within a family presupposes that its amino acid sequence is alignable with that of all members. Fifteen MT families [http://www.unizh.ch/˜mtpage/MT.html] have been characterized, each family being identified by its number and its taxonomic range: e.g., Family 1: vertebrate MTs.

This entry is a superfamily of metallothioneins, containing 3 families.

[QUICKGO ANNOTATION]

TABLE 8

| Function (1) | Function (8) |
|---|---|
| metal ion binding | cadmium ion binding activity (GO: 0046870) |
| activity (GO: 0046872) | zinc ion binding activity (GO: 0008270) |
| | manganese ion binding activity (GO: 0030145) |
| | molybdenum ion binding activity (GO: 0030151) |
| | mercury ion binding activity (GO: 0045340) |
| | nickel ion binding activity (GO: 0016151) |
| | iron ion binding activity (GO: 0005506) |
| | cooper ion binding activity (GO: 0005507) |

### Example 3

### Identification of Diseases Related to the Identified Domains

Using SMART and its OMIM curated human diseases associated with missense mutations within domains, in a first step, the human diseases associated with the domains found in UNC5H2d can be retrieved (tables 8 to 10). Complementary searches were performed with OMIM on already annotated domains and additional searches were done for non-annotated domains. In a second step, diseases that were associated with two or more domains found in UNC5H2d or diseases that were associated with a secreted protein (as UNC5H2d is a secreted protein) were retrieved.

[SMART Annotation]

3.1 First Step: Diseases Found Associated with Domains

1) SwissProt sequences and OMIM curated human diseases associated with missense mutations within the IG domain.

TABLE 9

| Protein | Disease |
|---|---|
| Tumor suppressor protein DCC precursor | (OMIM: 120470): Colorectal cancer |
| (Colorectal cancer suppressor). (SRS)(SMART) | |
| Poliovirus receptor precursor (CD155 antigen). | (OMIM: 173850): {Polio, |
| (SRS)(SMART) | susceptibility to} |
| Low affinity immunoglobulin gamma Fc region | (OMIM: 146790): {Lupus nephritis, |
| receptor II-A precursor (Fc-gamma RII-A) (FcRII-A) | susceptibility to} |
| (IgG Fc receptor II-A) (Fc-gamma-RIIA) (CD32) | (OMIM: 146740): {Lupus |
| (CDW32). (SRS)(SMART) | erythematosus, systemic, |
| | susceptibility} |
| (SRS)(SMART) | (OMIM: 152700): Neutropenia. |
| | alloimmune neonatal; {Viral |
| | infections, recurrent} |
| Intercellular adhesion molecule-1 precursor (ICAM- | (OMIM: 147840): {Malaria, cerebral, |
| 1) (Major group rhinovirus receptor) (CD54 | susceptibility to} |
| antigen). (SRS)(SMART) | (OMIM: 176943): Crouzon syndrome |
| (SRS)(SMART) | (OMIM: 123500): Jackson-Weiss |
| | syndrome |
| | (OMIM: 123150): Beare-Stevenson |
| | cutis gyrata syndrome |
| | (OMIM: 123790): Pfeiffer syndrome |
| | (OMIM: 101600): Apert syndrome |
| | (OMIM: 101200): Saethre-Chotzen |
| | syndrome |
| High affinity nerve growth factor receptor precursor | (OMIM: 191315): Insensitivity to |
| (EC 2.7.1.112) (TRK1 transforming tyrosine kinase | pain, congenital, with anhidrosis |
| protein) (p140-TrkA) (Trk-A). (SRS)(SMART) | (OMIM: 256800): Medullary thyroid |
| | carcinoma, familial |
| | (OMIM: 155240): |
| | (OMIM: 159440): Charcot-Marie- |
| | Tooth neuropathy-1B |
| Myelin P0 protein precursor (Myelin protein zero) | (OMIM: 118200): Dejerine-Sottas |
| (Myelin peripheral protein) (MPP). (SRS)(SMART) | disease, myelin P-zero-related |
| | (OMIM: 145900): Hypomyelination, |
| | congenital |
| | (OMIM: 308840): Hydrocephalus |
| | due to aqueductal stenosis |
| Neural cell adhesion molecule L1 precursor (N- | (OMIM: 307000): MASA syndrome |
| CAM L1) (CD171 antigen). (SRS)(SMART) | (OMIM: 303350): Spastic paraplegia |
| | (OMIM: 312900): |
| Myosin-binding protein C, cardiac-type (Cardiac | (OMIM: 600958): Cardiomyopathy, |
| MyBP-C) (C-protein, cardiac muscle isoform). | familial hypertrophic, 4 |
| (SRS)(SMART) | (OMIM: 115197): |
| T-cell surface glycoprotein CD4 precursor (T-cell | (OMIM: 186940): {Lupus |
| surface antigen T4/Leu-3). (SRS)(SMART) | erythematosus, susceptibility to} |

Search for other OMIM human diseases maybe associated with the IG domain: Wolf-Hirschhom syndrome, lymphoproliferative syndrome, acute myeloid leukemia, Bruton agammaglobulinemia, ataxia-telangiectasia, chronic myelogenous leukemia, achondroplasia, multiple sclerosis, Opitz syndrome, T-cell acute lymphocytic leukemia, dyskeratosis congenital, factor V deficiency, asthma, T-cell leukemia, IPEX, hypogammaglobulinemia, neutropenia, myeloid leukemia, antithrombin III deficiency, HIGM1, polycystic kidneys and Pfeiffer syndrome.

2) SwissProt sequences and OMIM curated human diseases associated with missense mutations within the IGc2 domain.

TABLE 10

| Protein | Disease |
|---|---|
| (SRS)(SMART) | (OMIM: 176943): Crouzon syndrome |
| | (OMIM: 123500): Jackson-Weiss |
| | syndrome |
| | (OMIM: 123150): Beare-Stevenson |
| | cutis gyrata syndrome |
| | (OMIM: 123790): Pfeiffer syndrome |
| | (OMIM: 101600): Apert syndrome |
| | (OMIM: 101200): Saethre-Chotzen |
| | syndrome |
| | (OMIM: 308840): Hydrocephalus |
| | due to aqueductal stenosis |
| Neural cell adhesion molecule L1 precursor (N- | (OMIM: 307000): MASA syndrome |
| CAM L1) (CD171 antigen). (SRS)(SMART) | (OMIM: 303350): Spastic paraplegia |
| | (OMIM: 312900): |
| Tumor suppressor protein DCC precursor | (OMIM: 120470): Colorectal cancer |
| (Colorectal cancer suppressor). (SRS)(SMART) | |
| Low affinity immunoglobulin gamma Fc region | (OMIM: 146790): {Lupus nephritis, |
| receptor II-A precursor (Fc-gamma RII-A) (FcRII- | susceptibility to} |
| A) (IgG Fc receptor II-A) (Fc-gamma-RIIA) (CD32) | |
| (CDW32). (SRS)(SMART) | |
| Myosin-binding protein C, cardiac-type (Cardiac | (OMIM: 600958): Cardiomyopathy, |
| MyBP-C) (C-protein, cardiac muscle isoform). | familial hypertrophic, 4 |
| (SRS)(SMART) | (OMIM: 115197): |
| | (OMIM: 146740): {Lupus |
| | erythematosus, systemic, |
| | susceptibility} |
| (SRS)(SMART) | (OMIM: 152700): Neutropenia, |
| | alloimmune neonatal; {Viral |
| | infections, recurrent} |

3) SwissProt sequences and OMIM curated human diseases associated with missense mutations within the TSP1 domain.

TABLE 11

| Protein | Disease |
|---|---|
| Properdin precursor (Factor P). | (OMIM: 312060): Properdin deficiency, X- |
| (SRS)(SMART) | linked |
| Complement component C6 precursor. | (OMIM: 217050): C6 deficiency; Combined |
| (SRS)(SMART) | C6/C7 deficiency |

Search for other OMIM human diseases maybe associated with the TSP1 domain: Ischemia, malignant neoplasms, Kaposi's sarcoma, orthostatic hypotension, factor V deficiency, thrombasthenia of Glanzmann, thrombotic thrombocytopenic purpura and Naegeli and Quebec platelet disorder.
4) Search for OMIM human diseases maybe associated with the death domain: Lymphoproliferative syndrome, glucocorticoid receptor defect, ectodermal dysplasia 1, Alzheimer disease, Leber optic atrophy, T-cell lymphoma/leukemia, Sezary syndrome, Lupus erythematosus, multiple sclerosis, APOE deficiency and hypertension.
5) Search for OMIM human diseases maybe associated with the ZU5 domain: Spherocytosis, Werner syndrome, log QT syndrome 4, hemolytic poikilocytic anemia, anemia, hyperbilirubinemia, elliptocytosis, poikilocytosis and Bare lymphocyte syndrome.
6) Search for OMIM human diseases maybe associated with the metallothionein domain: Myoclonic epilepsy of Unverricht and Lundborg, Menkes disease, hypercholesterolemia, hyperzincemia, cutaneous malignant melanoma, cutis laxa, pituitary dwarfism, hemophilia B, Wilson disease and tyrosine transaminase deficiency.
7) Search for OMIM human diseases maybe associated with the reeler domain: Miller-Dieker lissencephaly syndrome. Other diseases and conditions associated with the reeler domain are encompassed within the scope of this invention.
8) Search for OMIM human diseases maybe associated with the Plasmodium circumsporozoite protein signature domain: Malaria.
9) Search for OMIM human diseases maybe associated with the Factor 1 membrane attack complex: Complement component proteins deficiencies (e.g. C3 and C8 deficiencies), glomerulonephritis, lipodystrophy, factor D deficiency, proteinuria, Fukuyama congenital muscular dystrophy, muscle-eye-brain disease, hemolytic-uremic syndrome, asthma, Rheumatoid arthritis, H deficiency, schizophrenia, angioedema, nemaline myopathy 1, preselin dementia, Parkinsonism, Lupus erythematosus, trichothiodystrophy, ataxia telangiectasia, Tay-Sachs disease, colon cancer, Angelinan syndrome, hypercholesterolemia, Alzheimer's disease, Alport syndrome, Fanconi anemia, renal failure, atrioventricular septal defect, Niemanm-Pick disease, mucopolysaccharidosis type 1, specific granule deficiency, rhizomelic chondrodysplasia punctata, neurofibromatosis, dystrophia myotonica 1.
10) Search for OMIM human diseases maybe associated with the rhodanese signature domain: myopathy, Leber optic atrophy and so-called rhodanese deficiencies.
   3.2 Diseases that are Associated with Two or More Domains or Diseases that Were Associated with a Secreted Protein
   3.2.1 Lupus erythemasosus is found associated with the death domain, the immunoglobulin domain and the factor 1 membrane attack complex.
   3.2.2 Malaria is found associated with the immunoglobulin domain and the psalmodium circumsporozoite protein signature.
   3.2.3 The lymphoproliferative syndrome, T-cell leukemia and multiple sclerosis are found associated with the death and immunoglobulin domains.
   3.2.4 Factor V deficiencies are found associated with the thrombospondin type 1 motif and the immunoglobulin domain.
   3.2.5 Complement component proteins deficiencies (C3, C6, C8 and C6/7 combined deficiencies) are found associated with the factor 1 membrane attack complex and the thrombospondin type 1 motif.
   3.2.6 Asthma and ataxia telangiectasia are found associated with the immunoglobulin domain and the factor 1 membrane attack complex.
   3.2.7 Alzheimer disease is found associated with the factor 1 membrane attack complex and the death domain.
   3.2.8 Leber optic atrophy is associated with the death domain and the rhodanese signature.
   3.2.9 Hypercholesterolemia is found associated with the factor 1 membrane attack complex and the metallothionein domain.
   3.2.10 Myopathy is found associated with the rhodanese signature and the factor 1 membrane attack complex.
   3.2.11 Properdin deficiencies are found associated with a secreted protein (properdin precursor or factor P).

### Example 4

### Analysis of UNC5H2d Gene Expression Levels by TaqMan Analysis

Total RNA from each sample was reverse transcribed using the Superscript III First-Strand Synthesis System for RT-PCR (Invitrogen, Cat. No. 18080-051) in a final reaction volume of 20 μl. 2 μg of total RNA was combined with 50 ng random hexamer primers, 10 mM each of dATP, dGTP, dCTP, & dTTP, and DEPC-treated water in a volume of 10 μl. The mixture was incubated at 65° C. for 5 min then chilled on ice for 1 min. The following 10 μl cDNA synthesis mix was prepared in a separate tube: 2 μl 10× RT buffer, 4 μl 25 mM MgCl₂, 2 μl 0.1M DTT, 1 μl RnaseOUT™ (40 units/μl), and 1 μl SuperScript™ III RT enzyme (200 units/μl). The cDNA synthesis mix was added to the RNA/primer mixture, mixed gently and incubated at 25° C. for 10 min then at 50° C. for 50 min. The RT enzyme was then inactivated by incubating at 85° C. for 5 min. The mixture was chilled on ice and then 1 μl of E. coli Rnase H (2 units/μl) was added and the mixture incubated at 37° C. for 20 min. The mixture was chilled on ice and then diluted 1/250 with sterile water. Dilutions of the reverse transcriptase reaction were then subjected to real time PCR analysis on a TaqMan instrument (PE Biosystems 7700).

PCR primers for human UNC5H2d and the housekeeping control gene glyceraldehyde 3-phosphate dehydrogenase (GAPDH) were designed using the Primer Express software (PE Biosystems). The sequences of the primers are shown in Table 12. The specificity and the optimal primer concentration to use for the TaqMan analysis were determined by testing the UNC5H2d gene-specific primers on a series of dilutions of plasmid pCR-XL-TOPO-UNC5H2d (plasmid I.D. 13855). Potential genomic DNA contamination of the cDNA was excluded by performing PCR reactions using primers specific for GAPDH intronic sequence. The absence of non-specific amplification was controlled by analyzing the PCR products on 4% agarose gels to ensure a single band of the expected molecular weight was produced.

SYBR Green Real-Time PCR reactions were carried out in a reaction volume of 50 μl containing 25 μl SYBR Green PCR master mix (PE Biosystems) (to which 0.5 units AmpErase Uracil N-Glycosylase (UNG, PE Biosystems) had previously been added), 300 nM of each amplification primer, and 5 μl of RT-PCR product. Cycling was performed using the ABI PRISM 7700 (TaqMan) Detection System programmed as follows: 1 cycle of 50° C. for 2 min; 1 cycle of 95° C. for 10 min; 40 cycles of 95° C. for 15 sec, 60° C. for 1 min. Each reaction was carried out in duplicate and the results averaged.

The primer-specific regions of the reverse-transcribed cDNA samples were thus amplified and their cycle threshold (Ct) values determined. The Ct value for each cDNA sample was normalized to that of the housekeeping gene GAPDH as follows. The difference in expression level between the GAPDH gene and the UNC5H2d gene in each cDNA sample was expressed as a difference in Ct value, i.e. Delta (δ) Ct=Ct (GAPDH)−Ct (UNC5H2d). Results for each sample were then expressed as a fold difference in the number of cycles required for detectable UNC5H2d gene expression relative to that for GAPDH, according to the formula Fold Difference=2^{(-Ct)}. Finally, the expression level of the UNC5H2d gene in each cDNA sample was shown relative to the GAPDH gene expression level, where GAPDH expression level=100%, by dividing 100 by the Fold Difference for UNC5H2d. Results are shown in table 13.

TABLE 12
TaqMan PCR primer sequences

| | |
|---|---|
| Primer | Sequence (5′-3′) |

| | |
|---|---|
| UNC5H2d-121F | CGC TGC TCG ACT CTA AGA ACT G |
| UNC5H2d-183R | GTA GGA GCT GCG GGT TGC T |
| hGAPDH-F | CCA CCC ATG GCA AAT TCC |
| hGAPDH-R | GAT GGG ATT TCC ATT GAT GAC A |
| Intron-hGAPDH-F | CCT AGT CCC AGG GCT TTG ATT |
| Intron-hGAPDH-R | CTG TGC TCC CAC TCC TGA TTT |

TABLE 13
Expression of UNC5H2d in various human tissues as measured by RT-PCR (TaqMan).

| | | | | | Expression level of |
|---|---|---|---|---|---|
| | | | | | UNC5H2d relative to |
| | Ct | Ct | Delta | Fold | GAPDH expression |
| cDNA sample | (hGAPDH) | (UNC5H2d) | (δ) ct | difference | (GAPDH = 100%) |
| S76 Brain | 22.62 | 34.03 | −11.42 | 2730.60 | 0.04 |
| S77 Heart | 24.00 | 35.45 | −11.45 | 2797.65 | 0.04 |
| S78 Kidney | 22.93 | 34.49 | −11.56 | 3019.30 | 0.03 |
| S79 liver | 25.29 | 34.78 | −9.49 | 719.08 | 0.14 |
| S80 Lung | 25.23 | 34.08 | −8.86 | 463.04 | 0.22 |
| S81 Placenta | 24.61 | 34.55 | −9.95 | 985.70 | 0.10 |
| S82 skeletal Muscle | 19.42 | 34.72 | −15.30 | 40342.14 | 0.00 |
| S83 small intestine | 23.65 | 34.11 | −10.47 | 1413.44 | 0.07 |
| S84 Spleen | 25.70 | 35.70 | −10.01 | 1027.56 | 0.10 |
| S85 Thymus | 23.03 | 35.15 | −12.12 | 4435.87 | 0.02 |
| S86 Uterus | 24.29 | 34.85 | −10.57 | 1514.89 | 0.07 |
| S87 Bone Marrow | 25.12 | 35.06 | −9.94 | 978.89 | 0.10 |
| S88 Thyroid | 23.66 | 35.25 | −11.59 | 3072.08 | 0.03 |
| S89 Spinal cord | 22.11 | 34.30 | −12.20 | 4688.79 | 0.02 |
| S90 Cervix | 23.99 | 33.71 | −9.72 | 840.44 | 0.12 |
| S91 colon | 22.26 | 33.62 | −11.36 | 2628.46 | 0.04 |
| S92 ovary | 23.37 | 33.31 | −9.94 | 982.29 | 0.10 |
| S93 prostate | 21.28 | 32.95 | −11.67 | 3258.52 | 0.03 |
| S94 testis | 21.93 | 33.69 | −11.76 | 3468.27 | 0.03 |
| S95 skin | 24.08 | 34.32 | −10.24 | 1205.15 | 0.08 |
| S113 pancreas | 24.26 | 34.14 | −9.88 | 942.27 | 0.11 |
| S115 Salivary gland | 23.13 | 33.76 | −10.63 | 1579.22 | 0.06 |
| S116 Adrenal gland | 22.50 | 33.03 | −10.53 | 1478.58 | 0.07 |
| S117 Universal h- ref | 17.39 | 33.18 | −15.79 | 56462.29 | 0.00 |
| S119 Breast | 21.46 | 33.17 | −11.72 | 3361.76 | 0.03 |
| S120 Stomach | 22.16 | 34.20 | −12.04 | 4196.58 | 0.02 |
| S121 Fetal Kidney | 20.30 | 31.02 | −10.72 | 1680.88 | 0.06 |
| S122 Eye | 23.30 | 33.58 | −10.28 | 1243.34 | 0.08 |
| S123 Mammary gland | 24.98 | 34.03 | −9.06 | 531.90 | 0.19 |
| S124 Ovary | 20.19 | 33.49 | −13.30 | 10085.54 | 0.01 |
| S125 Pituitary gland | 22.65 | 34.07 | −11.43 | 2749.59 | 0.04 |
| S127 human lupus liver | 24.41 | 34.87 | −10.47 | 1413.44 | 0.07 |
| S128 human lupus Lung | 22.42 | 34.01 | −11.59 | 3082.75 | 0.03 |
| S129 human lupus Spleen | 23.63 | 33.99 | −10.36 | 1314.23 | 0.08 |
| S130 human lupus Kidney | 22.93 | 33.49 | −10.57 | 1514.89 | 0.07 |
| S131 cirrhosis liver | 21.86 | 33.71 | −11.85 | 3691.52 | 0.03 |
| S132 cirrhosis Lung | 18.01 | 33.41 | −15.40 | 43237.64 | 0.00 |
| S133 cirrhosis Spleen | 22.52 | 33.54 | −11.03 | 2083.80 | 0.05 |
| S134 cirrhosis Small intestine | 20.73 | 34.28 | −13.55 | 11952.29 | 0.01 |
| S135 kidney Tumor | 20.01 | 31.69 | −11.69 | 3292.57 | 0.03 |
| S136 Liver Tumor | 19.55 | 33.66 | −14.11 | 17682.08 | 0.01 |
| S137 Lung Tumor | 22.29 | 33.81 | −11.52 | 2936.74 | 0.03 |
| S138 colon Tumor | 19.52 | 33.69 | −14.18 | 18496.95 | 0.01 |
| S139 Breast Tumor | 20.05 | 33.53 | −13.48 | 11425.74 | 0.01 |
| S140 Fetal brain | 20.07 | 33.05 | −12.99 | 8107.27 | 0.01 |
| S141 Fetal spleen | 21.21 | 32.93 | −11.72 | 3373.43 | 0.03 |
| S142 Fetal Liver | 21.22 | 34.07 | −12.85 | 7383.04 | 0.01 |
| S143 Fetal Heart | 19.89 | 33.27 | −13.38 | 10660.59 | 0.01 |
| S144 Fetal Lung | 17.47 | 34.12 | −16.66 | 103194.03 | 0.00 |
| S145 Lymph Node | 22.32 | 34.18 | −11.86 | 3704.34 | 0.03 |
| S146 Adipose | 20.27 | 32.38 | −12.11 | 4405.23 | 0.02 |
| S147 Bladder | 21.24 | 33.26 | −12.03 | 4167.60 | 0.02 |
| S148 Appendix | 22.06 | 33.25 | −11.19 | 2336.28 | 0.04 |
| S149 Blood vessy Artery | 21.83 | 33.16 | −11.33 | 2574.36 | 0.04 |
| S150 Throast | 19.55 | 33.89 | −14.34 | 20738.16 | 0.00 |
| S151 Disease Brain | 22.56 | 31.98 | −9.42 | 685.02 | 0.15 |
| S1 Fibroblast AG1518 | 20.62 | 33.26 | −12.64 | 6360.83 | 0.02 |
| S2 Fibroblast Howard ab | 21.33 | 33.71 | −12.38 | 5330.30 | 0.02 |
| S3 Fibroblast Clark N | 20.33 | 32.47 | −12.14 | 4513.40 | 0.02 |
| S4 Fibroblast NF1 | 20.56 | 32.44 | −11.88 | 3769.09 | 0.03 |
| S5 Fibroblast NF2 | 21.72 | 33.60 | −11.88 | 3769.09 | 0.03 |
| S6 Fibroblast SSc N2 | 19.44 | 32.25 | −12.81 | 7181.15 | 0.01 |
| S7 Fibroblast SSCA2 | 19.11 | 32.45 | −13.34 | 10369.08 | 0.01 |
| S11 mixed RA2 | 23.17 | 33.82 | −10.65 | 1601.27 | 0.06 |
| S12 mixed RA3 | 21.94 | 33.28 | −11.34 | 2583.30 | 0.04 |
| S13 mixed OA1 | 25.83 | 34.18 | −8.35 | 326.29 | 0.31 |
| S15 Fibroblast LN1 | 20.03 | 33.08 | −13.05 | 8480.89 | 0.01 |
| S16 Fibroblast LAb1 | 18.66 | 32.32 | −13.66 | 12944.04 | 0.01 |
| S17 Fibroblast LN14 | 19.55 | 33.17 | −13.62 | 12590.08 | 0.01 |
| S18 Fibroblast LA13 | 19.83 | 32.97 | −13.14 | 9026.81 | 0.01 |
| S19 mixed OA4 | 24.45 | 34.11 | −9.66 | 809.00 | 0.12 |
| S27 mixed Lung A | 24.00 | 33.88 | −9.89 | 945.54 | 0.11 |
| S28 mixed Lung C | 23.26 | 33.82 | −10.56 | 1504.43 | 0.07 |
| S29 mixed Lung D | 23.41 | 33.68 | −10.27 | 1230.48 | 0.08 |
| S50 mixed colon 13224 | 22.14 | 33.32 | −11.18 | 2320.15 | 0.04 |
| S62 mixed small intestine normal int 21 | 22.26 | 33.93 | −11.67 | 3258.52 | 0.03 |
| S63 mixed small intestine normal int 23 | 22.18 | 33.40 | −11.22 | 2377.12 | 0.04 |
| S64 mixed small intestine Crohn's 8 | 25.18 | 34.50 | −9.33 | 641.36 | 0.16 |
| S65 mixed small intestine Crohn's 7 | 22.52 | 33.63 | −11.12 | 2217.93 | 0.05 |
| S67 mixed small intestine UC 18 | 23.65 | 33.37 | −9.72 | 843.36 | 0.12 |
| BN1 atherosclerotic plaque Z1 | 22.64 | 33.43 | −10.79 | 1764.45 | 0.06 |
| BN3 atherosclerotic plaque Z2 | 24.40 | 34.30 | −9.90 | 952.12 | 0.11 |
| BN5 atherosclerotic plaque Z3 | 24.43 | 34.11 | −9.69 | 823.14 | 0.12 |

Defining a threshold of Expression level of UNC5H2d relative to GAPDH expression of 0.05, the TaqMan expression results show that UNC5H2d is slightly expressed in the liver, lung, placenta, small intestine, spleen, uterus, bone marrow, cervix, ovary, skin, pancreas, salivary gland, adrenal gland, fetal kidney, eye, mammary gland, human lupus liver, human lupus spleen, human lupus kidney, cirrhosis spleen, in Alzheimer disease brain (S151 Disease Brain), rheumatoid arthritis synovium, osteoarthritic synovium, lung, in a Crohn's mixed small intestine (S64 and S65), in an ulcerative colitis mixed small intestine (S67), and in atherosclerotic plaques.

Dalvin S. et al. have shown that UNC5H2 is expressed in lung and might have a functional role in lung formation (Dalvin S. et al. Gene Expr Patterns. June 2003;3(3):279-83. “Expression of Netrin-1 and its two receptors DCC and UNC5H2 in the developing mouse lung.”).

Interestingly, there's no expression of UNC5H2d in breast tumor (S139, expression level is 0.01) compared with normal mammary gland (S123, expression level is 0.19). Likewise, UNC5H2d is undetectable in liver tumor (S136, expression level is 0.01) compared with normal liver (S79, expression level is 0.14), or in lung tumor (S137, expression level is 0.03) compared with normal lung (S80, expression level is 0.22). Thus, UNC5H2d, agonists or antagonists (e.g. antibodies) thereof may be useful in the treatment of cancer, for example breast tumor, liver tumor or lung tumor. Srinivasan et al. discuss the role of netrin-1 in mammary gland morphogenesis and its potential role in cancer (Srinivasan et al., Dev Cell. March 2003;4(3):371-82. “Netrin-1/neogenin interaction stabilizes multipotent progenitor cap cells during mammary gland morphogenesis.”).

There's also a consistent difference between the expression of UNC5H2d in Alzheimer disease brain (S151 Disease Brain, expression level is 0.15) compared with normal brain (S76, expression level is 00.04). As such, UNC5H2d, agonists or antagonists thereof (e.g. antibodies) might be useful in the treatment of neuropathologies like Alzheimer's disease. As explained before, Alzheimer's disease has also already been found associated with the factor 1 membrane attack complex and death domain, which are detected in UNC5H2d.

Finally, a difference is noted between the expression of UNC5H2d in normal small intestine (S62 and S63; expression level is 0.03-0.04) with a Crohn's small intestine (S64, expression level is 0.16) and an ulcerative colitis intestine (S67, expression level is 0.12). As such, UNC5H2d, agonists or antagonists thereof (e.g. antibodies) might be useful in the treatment of Crohn's disease and ulcerative colitis and more generally in treating or preventing inflammation.

### Example 5

### Autoimmunity/Inflammatory Assays

The following assays can be used to confirm the biological activity of an UNC5H2d polypeptide.

Assays Targeting T Lymphocyte Responses

Fas-Ligand-induced T cell death. This assay will reveal new modulators of receptor mediated cell death.

In this assay, T cell apoptosis is induced by stimulating Jurkat cells (a human T cell line) with recombinant 6 Histidine-tagged Fas Ligand combined with a monoclonal anti 6-his antibody. Death is quantified by release of LDH, a cytoplasmic enzyme released in the culture medium when cells are dying. The read out is a colorimetric assay read at 490 nm. T cells have been shown to be pathogenic in many autoimmune diseases, being able to control antigen-specific T cell death is a therapeutic strategy (e.g. anti-TNFα treatment in patient with Crohn's disease).

Human-MLR: proliferation and cytokine secretion. This cell-based assay measures the effects of novel proteins on lymphocyte proliferation and cytokine secretion or inhibition upon stimulation by PBMC from another donor (alloreactivity). These assay address antigen-specific T cell and antigen presenting cell functions, which are crucial cellular responses in any autoimmune diseases. Secreted cytokine (IL-2, 4, 5, 10, TNF-α and IFN-γ) are quantified by CBA.

Note: proliferation and cytokine secretion are independent responses.

Mouse-MLR: proliferation. This cell-based assay measures the effects of novel proteins on lymphocyte proliferation or inhibition of mouse spleen cells following stimulation by spleen cells from another donor (mouse strain). This cell-based assay measures the effect of novel proteins on T lymphocyte and antigen presenting cell responses and will be used to confirm activity of positives and hits identify in the h-MLR assays. This assay will be use to select proteins that will be tested in murine model of human diseases.

Human PBMC stimulated with the superantigen, TSST. Superantigens are strong modulators of the immune system affecting T cells. Superantigens influence immunologically mediated disorders such as IBD, inflammatory skin diseases like atopic dermatitis and psoriasis. In this cellular assay, we are specifically targeting T lymphocyte activation via the TCR but with different requirements than the T cell response to classical antigens, in particular in respect to co-stimulatory molecules.

Human PBMC stimulated with either ConA or PHA. These cell-based assays measure the effects of novel proteins on cytokine secretion induced by two different stimuli acting on different cells as measured by a cytokine bead array (CBA) assay (IL-2, IFN-γ, TNF-α, IL-5, IL4 and IL-10).

Most of cytokines can have dual actions, pro or anti-inflammatory, depending of the injury, milieu and cellular target. Any protein with the capability to modulate cytokine secretion may have a therapeutic potential (e.g. decreasing IFN-γ and TNF-α would be beneficial in Th1-mediated autoimmune disease in contrast decreasing IL4, IL-5 may be beneficial in Th2-mediated-diseases, inducing IL-10 would interesting in MS and SLE).

Assays Targeting Monocvte/Macrophages and Granulocyte Responses

Human PBMC stimulated with LPS. This cell-based assay measures the effects of novel proteins on cytokine secretion (IFN-γ, TNFα) induced by LPS acting on monocytes/macrophages and granulocytes.

Any protein with the capability to modulate IFN-γ and TNF-α secretion would be beneficial in Th1-mediated autoimmune diseases.

Assays Targeting Neutrophil Responses

Neutrophils are important in inflammation and autoimmune diseases such as Rheumatoid Arthritis. Leukocyte chemo-attractants such as IL-8 initiate a sequence of adhesive interactions between cells and the micro-vascular endothelium, resulting in activation, adhesion and finally migration of neutrophils. The tissue infiltration of neutrophils depends on a reorganisation of cytoskeleton elements associated with specific changes in cell morphology of these cells.

This cell-based assay measures the effect of novel proteins on cytoskeleton reorganization of human neutrophils.

Assays Targeting B Lymphocyte Responses

Autoantibodies as well as infiltrating B cells are thought to be important in the pathogenesis of various autoimmune diseases, such as systemic lupus erithematosus (SLE), rheumatoid arthritis (RA), Sjogren's syndrome and myasthenia gravis. Compelling evidence indicates that a disregulation in B cell homeostasis could affect immune tolerance leading to the inappropriate survival of autoreactive B cells producing pathogenic antibodies and sustained inflammation. The identification of new factors that play critical roles in the regulation of B cell proliferation, survival and differentiation following B cell receptor triggering are of high relevance in the development of novel therapies.

B cell proliferation. This cell-based assay measures the effect of novel proteins on B cell survival.

B cell co-stimulation. This cell-based assay measures the effect of novel proteins on B cell co-stimulation.

Assays Targeting Monocytes and Microglial Responses

THP-1 calcium flux. The Ca⁺-flux in THP1-cell assay measures the effects of novel proteins on their ability to trigger an intracellular calcium release (a generic second messenger events) from the endoplasmic reticulum.

Microglia cell proliferation (will be presented to the next IAC).

During proliferation of microglial progenitors, a number of colony-stimulating factors, including some cytokines, are known to play key roles. Among them, M-CSF is crucial for the final step of maturation of macrophages/microglia and is not replaceable by any other factor. The evaluation of this biological response may represent a way to influence the microglial activity and therefore an opportunity to identify molecules with therapeutic potential fro MS.

A cell-based assay was developed to measure the proliferative response of a microglia cell line to M-CSF. The feasibility and the robustness phases showed optimal results. This assay is in 96 well plates; non-radioactive substrate is required, easily automated.

### Example 6

### Neurological Assays Suitable for Exploration of the Biological Relevance of Proteins Function

The following assays can be used to confirm the biological activity of an UNC5H2d polypeptide. A number of neurological assays have been developed by the Applicant and are of use in the investigation of the biological relevance of protein function. Examples of neurological assays that have been developed by the Applicant include four types of assays. These are discussed below.

i. Oligodendrocytes-Based Assays

Oligodendrocytes are responsible for myelin formation in the CNS. In multiple sclerosis they are the first cells attacked and their loss leads to major behavioral impairment. In addition to curbing inflammation, enhancing the incomplete remyelination of lesions that occurs in MS has been proposed as a therapeutic strategy for MS. Like neurons, mature oligodendrocytes do not divide but the new oligodendrocytes can arise from progenitors. There are very few of these progenitor cells in adult brain and even in embryos the number of progenitor cells is inadequate for HTS.

Oli-neu is a murine cell line obtained by an immortalization of an oligodendrocyte precursor by the t-neu oncogene. They are well studied and accepted as a representative cell line to study young oligodendrocyte biology. These cells can be used in two types of assays.

One, to identify factors stimulating oligodendrocytes proliferation, and the other to find factors promoting their differentiation. Both events are key in the perspective of helping renewal and repairing demyelinating diseases.

Another possible cell line is the human cell line, MO3-13. MO3-13 results from the fusion of rabdo-myosarcoma cells with adult human oligodendrocytes. However these cells have a reduced ability to differentiate into oligodendrocytes and their proliferating rate is not sufficient to allow a proliferation assay. Nevertheless, they express certain features of oligodendrocytes and their morphology is well adapted to nuclear translocation studies.

Therefore this cell line can be used in assays based on nuclear translocation of three transcription factors, respectively NF-kB, Stat-1 and Stat-2. The Jak/Stats transcription pathway is a complex pathway activated by many factors such as IFN α, β, γ, cytokines (e.g. IL-2, IL-6; IL-5) or hormones (e.g. GH, TPO, EPO). The specificity of the response depends on the combination of activated Stats. For example, it is noticeable that IFN-β activates Stat1, 2 and 3 nuclear translocations meanwhile IFN-γ only activates Stat1. In the same way, many cytokines and growth factors induced NF-kB translocation. In these assays the goal is to get a picture of activated pathways for a given protein.

ii. Astrocytes-Based Assays

The biology of astrocytes is very complex, but two general states are recognized. In one state called quiescent, astrocytes regulate the metabolic and excitatory level of neurons by pumping glutamate and providing energetic substratum to neurons and oligodendrocytes. In the activated state, astrocytes produce chemokines and cytokines as well as nitric oxide. The first state could be considered as normal healthy while the second state occurs during inflammation, stroke or neurodegenerative diseases. When this activated state persists it should be regarded as a pathological state.

Many factors and many pathways are known to modulate astrocyte activation. In order to identify new factors modulating astrocyte activation U373 cells, a human cell line of astroglioma origin, can be used. NF-kB, c-Jun as well as Stats are signaling molecules known to play pivotal roles in astrocyte activation.

A series of screens based on the nuclear translocation of NF-κB, c-Jun and Stat1, 2 and 3 can be carried out. Prototypical activators of these pathways are IL-1b, IFN-beta or IFN-gamma. The goal is to identify proteins that could be used as therapeutics in the treatment of CNS diseases.

iii. Neurons-Based Assays

Neurons are very complex and diverse cells but they have all in common two things. First they are post-mitotic cells, secondly they are innervating other cells. Their survival is linked to the presence of trophic factors often produced by the innervated target cells. In many neurodegenerative diseases the lost of target innervation leads to cell body atrophy and apoptotic cell death. Therefore identification of trophic factors supplementing target deficiency is very important in treatment of neurodegenerative diseases.

In this perspective a survival assay using NS1 cells, a sub-clone of rat PC12 cells, can be performed. These cells have been used for years and a lot of neurobiology knowledge has been first acquired on these cells before being confirmed on primary neurons including the pathways involved in neuron survival and differentiation (MEK, PI3K, CREB). In contrast the N2A cells, a mouse neuroblastoma, are not responding to classical neurotrophic factors but Jun-kinase inhibitors prevent apoptosis induced by serum deprivation. Therefore assays on these two cell lines will help to find different types of “surviving promoting” proteins.

It is important to note that in the previous assays we will identify factors that promote both proliferation and differentiation. In order to identify factors specifically promoting neuronal differentiation, a NS1 differentiation assay based on neurite outgrowth can be used. Promoting axonal or dendritic sprouting in neurodegenerative diseases could be advantageous for two reasons. It will first help the degenerating neurons to re-grow and re-establish a contact with the target cells. Secondly, it will potentiate the so-called collateral sprouting from healthy fibers, a compensatory phenomenon that delays terminal phases of neurodegenerative such as Parkinson or AD.

iv. Endothelial Cells-Based Assays

The blood brain barrier (BBB) between brain and vessels is responsible of differences between cortical spinal fluid and serum compositions. The BBB results from a tight contact between endothelial cells and astrocytes. It maintains an immunotolerant status by preventing leukocytes penetration in brain, and allows the development of two parallels endocrine systems using the same intracellular signaling pathways. However, in many diseases or traumas, the BBB integrity is altered and leukocytes as well as serum proteins enter the brain inducing neuroinflammation. There is no easy in vitro model of BBB, but cultures of primary endothelial cells such as human embryonic umbilical endothelial cells (HUVEC) could mimic some aspect of BBB biology. For example, BBB leakiness could be induced by proteins stimulating intracellular calcium release. In the perspective of identifying proteins that modulate BBB integrity, a calcium mobilization assay with or without thrombin can be performed on HUVEC.

### Example 7

### Mouse Models

The following mouse models can be used to confirm biological activity of an UNC5H2d polypeptide as disclosed herein. The mouse models are described in Chu et al. (Gene-engineered models for genetic manipulation and functional analysis of the cardiovascular system in mice. Chang Gung Med J. December 2003;26(12):868-78.), in Ma et al. (Neurocardiovascular regulation in mice: experimental approaches and novel findings. Clin Exp Pharmacol Physiol. November2003;30(11):885-93.) or Svenson et al. (Invited review: Identifying new mouse models of cardiovascular disease: a review of high-throughput screens of mutagenized and inbred strains. J Appl Physiol. April 2003;94(4):1650-9; discussion 1673.).

Alternatively, the following mouse model can be used to confirm the biological activity of an UNC5H2d polypeptide as disclosed in Lu et al. (Lu et al. Nature. Nov. 11, 2004;432(7014):179-86. Epub Oct. 27, 2004.) by assaying UNC5H2d polypeptides in vascular development, specifically by measuring accumulation of blood in the venous circulation and fluid in the pericardial activity, or measuring peripheral resistance resulting from modified arterial vasculature, or measuring capillaries thickness and branching in hindbrains, or measuring filopodial extension from endothelial tip cells, or characterizing intersegmental blood vessels (ISVs) trajectory phenotypes and more generally vessel-branching defects.

### REFERENCES

1) Engels et al., 1989, Angew. Chem. Int. Ed. 28:716-34.
2) Hong K, Hinck L, Nishiyama M, Poo M M, Tessier-Lavigne M, Stein E. A ligand-gated association between cytoplasmic domains of UNC5 and DCC family receptors converts netrin-induced growth cone attraction to repulsion.
   Cell. Jun. 25, 1999; 97(7):927-41.
3) Io Davis et al., 1996, Cell, 87:1161-69.
4) Kitts et al., 1993, Biotechniques, 14:810-17.
5) Komatsuzaki K, Dalvin S, Kinane T B. Modulation of G(ialpha(2)) signaling by the axonal guidance molecule UNC5H2.
Biochem Biophys Res Commun. Oct. 4, 2002; 297(4): 898-905.
6) Lathe R, et al. J. Molec. Biol. 183:1-12 (1985).
7) Lu et al. Nature. Nov. 11, 2004;432(7014):179-86. Epub Oct. 27, 2004.
8) Maniatis T. et al. Molecular Cloning: A laboratory manual, Cold Spring Harbor Press, Cold Spring Harbor, N.Y., 1982.
9) Marston et al., 1990, Meth. Enz., 182:264-75.
10) Meinkoth J, Wahl G. Hybridization of nucleic acids immobilized on solid supports. Anal Biochem. May 1, 1984; 138(2):267-84.
11) Mitchell K J, Doyle J L, Serafini T, Kennedy T E, Tessier-Lavigne M, Goodman C S, Dickson B J. Genetic analysis of Netrin genes in Drosophila: Netrins guide CNS commissural axons and peripheral motor axons.
Neuron. August 2006; 17(2):203-15.
12) Roberts et al., 1997, Proc. Natl. Acad. Sci. U.S.A. 94:12297-303.
13) Sambrook et al., Molecular Cloning: A Laboratory Manual (Cold Spring Harbor Laboratory Press, 1989).
14) Srinivasan K, Strickland P, Valdes A, Shin G C, Hinck L. Netrin-1/neogenin interaction stabilizes multipotent progenitor cap cells during mammary gland morphogenesis.
   Dev Cell. March2003; 4(3): 371-82.
15) Tanikawa C, Matsuda K, Fukuda S, Nakamura Y, Arakawa H. p53RDL1 regulates p53-dependent apoptosis.
Nat Cell Biol. March 2003; 5(3): 216-23.
16) Thiebault K, Mazelin L, Pays L, Llambi F, Joly M O, Scoazec J Y, Saurin J C, Romeo G, Mehlen P. The netrin-1 receptors UNC5H are putative tumor suppressors controlling cell death commitment.
Proc Natl Acad Sci USA. Apr. 1, 2003; 100(7): 4173-8.
17) Williams M E, Strickland P, Watanabe K, Hinck L. UNC5H1 Induces Apoptosis via Its Juxtamembrane Region through an Interaction with NRAGE.
J Biol Chem. May 9, 2003; 278(19): 17483-90.
18) U.S. Pat. No. 5,824,469; University of Washington.
19) PCT/IJS98/20094 (W099/15650), Athersys.
20) WO 94/20069; Amgen inc.

1-44. (canceled)

45. A composition of matter comprising:
(a) an isolated nucleic acid molecule comprising a nucleotide sequence selected from:
(i) the nucleotide sequence as set forth in any of SEQ ID NO:1 or SEQ ID NO:3;
(ii) a nucleotide sequence encoding the polypeptide as set forth in any of SEQ ID NO:2, or SEQ ID NO:4, or SEQ ID NO:11, or encoding a polypeptide which exhibits at least about 85% identity to the polypeptide as set forth in any of SEQ ID NO:2, or SEQ ID NO:4, or SEQ ID NO:11, wherein the encoded polypeptide has a biological activity of the polypeptide set forth in any of SEQ ID NO:2, or SEQ ID NO:4, or SEQ ID NO:11, provided that such polypeptide sequence is not identical to any of SEQ ID NO:5, or SEQ ID NO:6, or SEQ ID NO:7;
(iii) a nucleotide sequence encoding the mature form of the polypeptide whose sequence is recited in SEQ ID NO:2 (SEQ ID NO:4);
(iv) a nucleotide sequence encoding the histidine tag form of the polypeptide whose sequence is recited in SEQ ID NO:2 (SEQ ID NO:11);
(v) a nucleotide sequence which hybridizes under stringent conditions with any of SEQ ID NO:1 or SEQ ID NO:3, or exhibit at least about 85% identity over a stretch of at least about 30 nucleotides, with a nucleic acid consisting of any of SEQ ID NO:1 or SEQ ID NO:3, or a complement of said DNA sequences, provided that such polynucleotide sequence is not identical to any of SEQ ID NO:8, or SEQ ID NO:9, or SEQ ID NO:10; or
(vi) a nucleotide sequence encoding a polypeptide as set forth in any of SEQ ID NO:2, or SEQ ID NO:4, or SEQ ID NO:11, with at least one conservative amino acid substitution, wherein the encoded polypeptide has a biological activity of the polypeptide set forth in any of SEQ ID NO:2, or SEQ ID NO:4, or SEQ ID NO:11;
(b) a vector comprising a nucleotide sequence comprising:
(i) the nucleotide sequence as set forth in any of SEQ ID NO:1 or SEQ ID NO:3;
(ii) a nucleotide sequence encoding the polypeptide as set forth in any of SEQ ID NO:2, or SEQ ID NO:4, or SEQ ID NO:11, or encoding a polypeptide which exhibits at least about 85% identity to the polypeptide as set forth in any of SEQ ID NO:2, or SEQ ID NO:4, or SEQ ID NO:11, wherein the encoded polypeptide has a biological activity of the polypeptide set forth in any of SEQ ID NO:2, or SEQ ID NO:4, or SEQ ID NO:11, provided that such polypeptide sequence is not identical to any of SEQ ID NO:5, or SEQ ID NO:6, or SEQ ID NO:7;
(iii) a nucleotide sequence encoding the mature form of the polypeptide whose sequence is recited in SEQ ID NO:2 (SEQ ID NO:4);
(iv) a nucleotide sequence encoding the histidine tag form of the polypeptide whose sequence is recited in SEQ ID NO:2 (SEQ ID NO:11);
(v) a nucleotide sequence which hybridizes under stringent conditions with any of SEQ ID NO:1 or SEQ ID NO:3, or exhibit at least about 85% identity over a stretch of at least about 30 nucleotides, with a nucleic acid consisting of any of SEQ ID NO:1 or SEQ ID NO:3, or a complement of said DNA sequences, provided that such polynucleotide sequence is not identical to any of SEQ ID NO:8, or SEQ ID NO:9, or SEQ ID NO:10; or
(vi) a nucleotide sequence encoding a polypeptide as set forth in any of SEQ ID NO:2, or SEQ ID NO:4, or SEQ ID NO:11, with at least one conservative amino acid substitution, wherein the encoded polypeptide has a biological activity of the polypeptide set forth in any of SEQ ID NO:2, or SEQ ID NO:4, or SEQ ID NO:11;
(c) a host cell comprising a vector comprising a nucleotide sequence comprising:
(i) the nucleotide sequence as set forth in any of SEQ ID NO:1 or SEQ ID NO:3;
(ii) a nucleotide sequence encoding the polypeptide as set forth in any of SEQ ID NO:2, or SEQ ID NO:4, or SEQ ID NO:11, or encoding a polypeptide which exhibits at least about 85% identity to the polypeptide as set forth in any of SEQ ID NO:2, or SEQ ID NO:4, or SEQ ID NO:11, wherein the encoded polypeptide has a biological activity of the polypeptide set forth in any of SEQ ID NO:2, or SEQ ID NO:4, or SEQ ID NO:11, provided that such polypeptide sequence is not identical to any of SEQ ID NO:5, or SEQ ID NO:6, or SEQ ID NO:7;
(iii) a nucleotide sequence encoding the mature form of the polypeptide whose sequence is recited in SEQ ID NO:2 (SEQ ID NO:4);
(iv) a nucleotide sequence encoding the histidine tag form of the polypeptide whose sequence is recited in SEQ ID NO:2 (SEQ ID NO:11);
(v) a nucleotide sequence which hybridizes under stringent conditions with any of SEQ ID NO:1 or SEQ ID NO:3, or exhibit at least about 85% identity over a stretch of at least about 30 nucleotides, with a nucleic acid consisting of any of SEQ ID NO:1 or SEQ ID NO:3, or a complement of said DNA sequences, provided that such polynucleotide sequence is not identical to any of SEQ ID NO:8, or SEQ ID NO:9, or SEQ ID NO:10; or
(vi) a nucleotide sequence encoding a polypeptide as set forth in any of SEQ ID NO:2, or SEQ ID NO:4, or SEQ ID NO:11, with at least one conservative amino acid substitution, wherein the encoded polypeptide has a biological activity of the polypeptide set forth in any of SEQ ID NO:2, or SEQ ID NO:4, or SEQ ID NO:11;
(d) an isolated polypeptide comprising:
(i) the amino acid sequence as set forth in any of SEQ ID NO:2, or SEQ ID NO:4, or SEQ ID NO:11;
(ii) the amino acid sequence as set forth in any of SEQ ID NO:2, or SEQ ID NO:4, or SEQ ID NO:11 with at least one conservative amino acid substitution in the amino acid sequence as set forth in any of SEQ ID NO:2, or SEQ ID NO:4, or SEQ ID NO:11, wherein the polypeptide has a biological activity of the polypeptide set forth in any of SEQ ID NO:2, or SEQ ID NO:4, or SEQ ID NO:11;
(iii) the amino acid sequence of SEQ ID NO:4;
(iv) the amino acid sequence of SEQ ID NO:2;
(v) a polypeptide fragment spanning from amino acids 1 to 355 of SEQ ID NO:2;
(vi) a polypeptide fragment spanning from amino acids 26 to 355 of SEQ ID NO:2;
(vii) a polypeptide fragment consisting of the two immunoglobulin domains and of the two thrombospondin domains of SEQ ID NO:2;
(viii) a fragment consisting of the two immunoglobulin domains of SEQ ID NO:2;
(ix) a polypeptide fragment consisting of the two thrombospondin domains of SEQ ID NO:2;
(x) a fusion protein comprising a histidine tag and SEQ ID NO:2, SEQ ID NO:4, or SEQ ID NO:11; or
(xi) a polypeptide encoded by a nucleic acid molecule as set forth in (a);
(e) an isolated binding agent or fragment thereof that specifically binds a polypeptide comprising:
(i) the amino acid sequence as set forth in any of SEQ ID NO:2, or SEQ ID NO:4, or SEQ ID NO:11;
(ii) the amino acid sequence as set forth in any of SEQ ID NO:2, or SEQ ID NO:4, or SEQ ID NO:11 with at least one conservative amino acid substitution in the amino acid sequence as set forth in any of SEQ ID NO:2, or SEQ ID NO:4, or SEQ ID NO:11, wherein the polypeptide has a biological activity of the polypeptide set forth in any of SEQ ID NO:2, or SEQ ID NO:4, or SEQ ID NO:11;
(iii) the amino acid sequence of SEQ ID NO:4;
(iv) the amino acid sequence of SEQ ID NO:2;
(v) a polypeptide fragment spanning from amino acids 1 to 3 55 of SEQ ID NO:2;
(vi) a polypeptide fragment spanning from amino acids 26 to 355 of SEQ ID NO:2;
(vii) a polypeptide fragment consisting of the two immunoglobulin domains and of the two thrombospondin domains of SEQ ID NO:2;
(viii) a fragment consisting of the two immunoglobulin domains of SEQ ID NO:2;
(ix) a polypeptide fragment consisting of the two thrombospondin domains of SEQ ID NO:2;
(x) a fusion protein comprising a histidine tag and SEQ ID NO:2, SEQ ID NO:4, or SEQ ID NO:11; or
(xi) a polypeptide encoded by a nucleic acid molecule as set forth in (a);
(f) a hybridoma producing a selective binding agent or a fragment thereof that specifically binds a polypeptide comprising:
(i) the amino acid sequence as set forth in any of SEQ ID NO:2, or SEQ ID NO:4, or SEQ ID NO:11;
(ii) the amino acid sequence as set forth in any of SEQ ID NO:2, or SEQ ID NO:4, or SEQ ID NO:11 with at least one conservative amino acid substitution in the amino acid sequence as set forth in any of SEQ ID NO:2, or SEQ ID NO:4, or SEQ ID NO:11, wherein the polypeptide has a biological activity of the polypeptide set forth in any of SEQ ID NO:2, or SEQ ID NO:4, or SEQ ID NO:11;
(iii) the amino acid sequence of SEQ ID NO:4;
(iv) the amino acid sequence of SEQ ID NO:2;
(v) a polypeptide fragment spanning from amino acids 1 to 355 of SEQ ID NO:2;
(vi) a polypeptide fragment spanning from amino acids 26 to 355 of SEQ ID NO:2;
(vii) a polypeptide fragment consisting of the two immunoglobulin domains and of the two thrombospondin domains of SEQ ID NO:2;
(g) a composition comprising a pharmaceutically acceptable formulation agent and nucleic acid molecule comprising:
(i) the nucleotide sequence as set forth in any of SEQ ID NO:1 or SEQ ID NO:3;
(ii) a nucleotide sequence encoding the polypeptide as set forth in any of SEQ ID NO:2, or SEQ ID NO:4, or SEQ ID NO:11, or encoding a polypeptide which exhibits at least about 85% identity to the polypeptide as set forth in any of SEQ ID NO:2, or SEQ ID NO:4, or SEQ ID NO:11, wherein the encoded polypeptide has a biological activity of the polypeptide set forth in any of SEQ ID NO:2, or SEQ ID NO:4, or SEQ ID NO:11, provided that such polypeptide sequence is not identical to any of SEQ ID NO:5, or SEQ ID NO:6, or SEQ ID NO:7;
(iii) a nucleotide sequence encoding the mature form of the polypeptide whose sequence is recited in SEQ ID NO:2 (SEQ ID NO:4);
(iv) a nucleotide sequence encoding the histidine tag form of the polypeptide whose sequence is recited in SEQ ID NO:2 (SEQ ID NO:11);
(v) a nucleotide sequence which hybridizes under stringent conditions with any of SEQ ID NO:1 or SEQ ID NO:3, or exhibit at least about 85% identity over a stretch of at least about 30 nucleotides, with a nucleic acid consisting of any of SEQ ID NO:1 or SEQ ID NO:3, or a complement of said DNA sequences, provided that such polynucleotide sequence is not identical to any of SEQ ID NO:8, or SEQ ID NO:9, or SEQ ID NO:10; or
(vi) a nucleotide sequence encoding a polypeptide as set forth in any of SEQ ID NO:2, or SEQ ID NO:4, or SEQ ID NO:11, with at least one conservative amino acid substitution, wherein the encoded polypeptide has a biological activity of the polypeptide set forth in any of SEQ ID NO:2, or SEQ ID NO:4, or SEQ ID NO:11; or
(h) a composition comprising a pharmaceutically acceptable formulation agent and:
(i) the amino acid sequence as set forth in any of SEQ ID NO:2, or SEQ ID NO:4, or SEQ ID NO:11;
(ii) the amino acid sequence as set forth in any of SEQ ID NO:2, or SEQ ID NO:4, or SEQ ID NO:11 with at least one conservative amino acid substitution in the amino acid sequence as set forth in any of SEQ ID NO:2, or SEQ ID NO:4, or SEQ ID NO:11, wherein the polypeptide has a biological activity of the polypeptide set forth in any of SEQ ID NO:2, or SEQ ID NO:4, or SEQ ID NO:11;
(iii) the amino acid sequence of SEQ ID NO:4;
(iv) the amino acid sequence of SEQ ID NO:2;
(v) a polypeptide fragment spanning from amino acids 1 to 355 of SEQ ID NO:2;
(vi) a polypeptide fragment spanning from amino acids 26 to 355 of SEQ ID NO:2;
(vii) a polypeptide fragment consisting of the two immunoglobulin domains and of the two thrombospondin domains of SEQ ID NO:2;
(viii) a fragment consisting of the two immunoglobulin domains of SEQ ID NO:2;
(ix) a polypeptide fragment consisting of the two thrombospondin domains of SEQ ID NO:2;
(x) a fusion protein comprising a heterologous sequence and SEQ ID NO:2, SEQ ID NO:4, or SEQ ID NO:11; or
(xi) a polypeptide encoded by a nucleic acid molecule as set forth in (a).

46. The composition of matter according to claim 45, wherein the selective binding agent or fragment thereof specifically binds the polypeptide comprising the amino acid sequence as set forth in any of SEQ ID NO:2, SEQ ID NO:4, SEQ ID NO:11, or a fragment thereof.

47. The composition of matter according to claim 46, wherein said selective binding agent is an antibody or an antigen binding fragment thereof.

48. The composition of matter according to claim 47, wherein said antibody or antigen binding fragment thereof is a chimeric or humanized antibody.

49. The composition of matter according to claim 45, wherein the pharmaceutically acceptable formulation agent of said composition is a carrier, adjuvant, solubilizer, stabilizer, or anti oxidant.

50. The composition of matter according to claim 45, wherein said composition comprises the amino acid sequence as set forth in any of SEQ ID NO:2, or SEQ ID NO:4, or SEQ ID NO:11.

51. The composition of matter according to claim 45, wherein said polypeptide is derivatized.

52. The composition of matter according to claim 51, wherein said derivatized polypeptide is covalently modified with a water-soluble polymer.

53. The composition of matter according to claim 52, wherein the water-soluble polymer is selected from the group consisting of polyethylene glycol, mono-methoxy polyethylene glycol, dextran, cellulose, poly-(N-vinyl pyrrolidone) polyethylene glycol, propylene glycol homopolymers, polypropylene oxide/ethylene oxide copolymers, polyoxyethylated polyols, and polyvinyl alcohol.

54. The composition of matter according to claim 45, wherein said vector is a viral vector.

55. The composition of matter according to claim 45, wherein the heterologous amino acid sequence is an IgG constant domain or fragment thereof.

56. The composition of matter according to claim 45, wherein the percent identity of said nucleic acid molecule is determined using a computer program selected from the group consisting of GAP, BLASTN, FASTA, BLASTA, BLASTX, BestFit, and the SmithWaterman algorithm.

57. The composition of matter according to claim 45, wherein said host is a eukaryotic cell.

58. The composition of matter according to claim 45, wherein said host cell is a prokaryotic cell.

59. A process of producing an UNC5H2d polypeptide comprising culturing a host cell under suitable conditions to express the polypeptide, and optionally isolating the polypeptide from the culture, wherein said host cell comprises a vector comprising a nucleotide sequence comprising:
(a) the nucleotide sequence as set forth in any of SEQ ID NO:1 or SEQ ID NO:3;
(b) a nucleotide sequence encoding the polypeptide as set forth in any of SEQ ID NO:2, or SEQ ID NO:4, or SEQ ID NO:11, or encoding a polypeptide which exhibits at least about 85% identity to the polypeptide as set forth in any of SEQ ID NO:2, or SEQ ID NO:4, or SEQ ID NO:11, wherein the encoded polypeptide has a biological activity of the polypeptide set forth in any of SEQ ID NO:2, or SEQ ID NO:4, or SEQ ID NO:11, provided that such polypeptide sequence is not identical to any of SEQ ID NO:5, or SEQ ID NO:6, or SEQ ID NO:7;
(c) a nucleotide sequence encoding the mature form of the polypeptide whose sequence is recited in SEQ ID NO:2 (SEQ ID NO:4);
(d) a nucleotide sequence encoding the histidine tag form of the polypeptide whose sequence is recited in SEQ ID NO:2 (SEQ ID NO:11);
(e) a nucleotide sequence which hybridizes under stringent conditions with any of SEQ ID NO:1 or SEQ ID NO:3, or exhibit at least about 85% identity over a stretch of at least about 30 nucleotides, with a nucleic acid consisting of any of SEQ ID NO:1 or SEQ ID NO:3, or a complement of said DNA sequences, provided that such polynucleotide sequence is not identical to any of SEQ ID NO:8, or SEQ ID NO:9, or SEQ ID NO:10; or
(f) a nucleotide sequence encoding a polypeptide as set forth in any of SEQ ID NO:2, or SEQ ID NO:4, or SEQ ID NO:11, with at least one conservative amino acid substitution, wherein the encoded polypeptide has a biological activity of the polypeptide set forth in any of SEQ ID NO:2, or SEQ ID NO:4, or SEQ ID NO:11.

60. The process according to claim 59, wherein the nucleic acid molecule comprises a promoter DNA other than the promoter DNA for the native UNC5H2d polypeptide operatively linked to the DNA encoding the UNC5H2d polypeptide.

61. The process according to claim 60, wherein said method determines whether a compound inhibits UNC5H2d polypeptide activity or UNC5H2d polypeptide production and comprises exposing a host cell to a compound and measuring UNC5H2d polypeptide activity or UNC5H2d polypeptide production in said host cell, wherein said host cell comprises a vector comprising a nucleotide sequence comprising:
(a) the nucleotide sequence as set forth in any of SEQ ID NO:1 or SEQ ID NO:3;
(b) a nucleotide sequence encoding the polypeptide as set forth in any of SEQ ID NO:2, or SEQ ID NO:4, or SEQ ID NO:11, or encoding a polypeptide which exhibits at least about 85% identity to the polypeptide as set forth in any of SEQ ID NO:2, or SEQ ID NO:4, or SEQ ID NO:11, wherein the encoded polypeptide has a biological activity of the polypeptide set forth in any of SEQ ID NO:2, or SEQ ID NO:4, or SEQ ID NO:11, provided that such polypeptide sequence is not identical to any of SEQ ID NO:5, or SEQ ID NO:6, or SEQ ID NO:7;
(c) a nucleotide sequence encoding the mature form of the polypeptide whose sequence is recited in SEQ ID NO:2 (SEQ ID NO:4);
(d) a nucleotide sequence encoding the histidine tag form of the polypeptide whose sequence is recited in SEQ ID NO:2 (SEQ ID NO:11);
(e) a nucleotide sequence which hybridizes under stringent conditions with any of SEQ ID NO:1 or SEQ ID NO:3, or exhibit at least about 85% identity over a stretch of at least about 30 nucleotides, with a nucleic acid consisting of any of SEQ ID NO:1 or SEQ ID NO:3, or a complement of said DNA sequences, provided that such polynucleotide sequence is not identical to any of SEQ ID NO:8, or SEQ ID NO:9, or SEQ ID NO:10; or
(f) a nucleotide sequence encoding a polypeptide as set forth in any of SEQ ID NO:2, or SEQ ID NO:4, or SEQ ID NO:11, with at least one conservative amino acid substitution, wherein the encoded polypeptide has a biological activity of the polypeptide set forth in any of SEQ ID NO:2, or SEQ ID NO:4, or SEQ ID NO:11.

62. The process according to claim 61, wherein said host cell is prokaryotic or eukaryotic.

63. An isolated polypeptide produced by the process of claim 59.

64. A method of using the composition of matter according to claim 45 for: a) determining whether a compound inhibits UNC5H2d polypeptide activity or UNC5H2d polypeptide production; b) detecting or quantitating the amount of the UNC5H2d polypeptide; or c) treating or ameliorating a medical condition.

65. The method according to claim 64, wherein said method detects or quantitates the amount of UNC5H2d polypeptide using an anti-UNC5H2d antibody or antigen binding fragment thereof, a chimeric anti-UNC5H2d antibody or antigen binding fragment thereof, or a humanized anti-UNC5H2d antibody or antigen binding fragment thereof.

66. The method according to claim 64, wherein said method treats or ameliorates a medical condition or disease and comprises administering to a patient the polypeptide comprising:
(a) the amino acid sequence as set forth in any of SEQ ID NO:2, or SEQ ID NO:4, or SEQ ID NO:11;
(b) the amino acid sequence as set forth in any of SEQ ID NO:2, or SEQ ID NO:4, or SEQ ID NO:11 with at least one conservative amino acid substitution in the amino acid sequence as set forth in any of SEQ ID NO:2, or SEQ ID NO:4, or SEQ ID NO:11, wherein the polypeptide has a biological activity of the polypeptide set forth in any of SEQ ID NO:2, or SEQ ID NO:4, or SEQ ID NO:11;
(c) the amino acid sequence of SEQ ID NO:4;
(d) the amino acid sequence of SEQ ID NO:2;
(e) a polypeptide fragment spanning from amino acids 1 to 355 of SEQ ID NO:2;
(f) a polypeptide fragment spanning from amino acids 26 to 355 of SEQ ID NO:2;
(g) a polypeptide fragment consisting of the two immunoglobulin domains and of the two thrombospondin domains of SEQ ID NO:2;
(h) a fragment consisting of the two immunoglobulin domains of SEQ ID NO:2;
(i) a polypeptide fragment consisting of the two thrombospondin domains of SEQ ID NO:2;
(j) a fusion protein comprising a histidine tag and SEQ ID NO:2, SEQ ID NO:4, or SEQ ID NO:11; or
(k) a polypeptide encoded by a nucleic acid molecule comprising:
(i) the nucleotide sequence as set forth in any of SEQ ID NO:1 or SEQ ID NO:3;
(ii) a nucleotide sequence encoding the polypeptide as set forth in any of SEQ ID NO:2, or SEQ ID NO:4, or SEQ ID NO:11, or encoding a polypeptide which exhibits at least about 85% identity to the polypeptide as set forth in any of SEQ ID NO:2, or SEQ ID NO:4, or SEQ ID NO:11, wherein the encoded polypeptide has a biological activity of the polypeptide set forth in any of SEQ ID NO:2, or SEQ ID NO:4, or SEQ ID NO:11, provided that such polypeptide sequence is not identical to any of SEQ ID NO:5, or SEQ ID NO:6, or SEQ ID NO:7;
(iii) a nucleotide sequence encoding the mature form of the polypeptide whose sequence is recited in SEQ ID NO:2 (SEQ ID NO:4);
(iv) a nucleotide sequence encoding the histidine tag form of the polypeptide whose sequence is recited in SEQ ID NO:2 (SEQ ID NO:11);
(v) a nucleotide sequence which hybridizes under stringent conditions with any of SEQ ID NO:1 or SEQ ID NO:3, or exhibit at least about 85% identity over a stretch of at least about 30 nucleotides, with a nucleic acid consisting of any of SEQ ID NO:1 or SEQ ID NO:3, or a complement of said DNA sequences, provided that such polynucleotide sequence is not identical to any of SEQ ID NO:8, or SEQ ID NO:9, or SEQ ID NO:10; or
(vi) a nucleotide sequence encoding a polypeptide as set forth in any of SEQ ID NO:2, or SEQ ID NO:4, or SEQ ID NO:11, with at least one conservative amino acid substitution, wherein the encoded polypeptide has a biological activity of the polypeptide set forth in any of SEQ ID NO:2, or SEQ ID NO:4, or SEQ ID NO:11.

67. The method according to claim 66, wherein the disease is selected from cancer, cardiovascular disorder and/or hematology-related disorder.

68. The method according to claim 67, wherein the cardiovascular disorder is selected from cardiac and respiratory arrest, valvular heart disease, arterial hypertension, endocarditis, orthostatic hypotension, syncope, pericardial disease, arteriosclerosis, cardiac tumor, coronary artery disease, disease of the aorta and its branches, heart failure, peripheral vascular disorder, shock, athletic heart syndrome or arrhythmia.

69. The method according to claim 67, wherein the hematology-related disorder is selected from anemia, histiocytic syndrome, iron overload related disorder, leukemia, lymphoma, myeloproliferative disorder, plasma cell dyscrasia, hemostasis and coagulation disorder, disorder of the spleen, thrombotic disorder, platelet disorder, vascular bleeding disorder, leukopenia, lymphocytopenia or AIDS-associated hematologic disorder and malignancy.
